SEQUENCE LISTING (91 sequences)

SEQ ID NO: 1 - protein, 298 aa, Homo sapiens
MVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 2 - protein, 297 aa, Homo sapiens
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 3 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 4 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1916 aa, synthetic construct
MKRTADGSEFESPKKKRKVVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQALGGDSGGSGGSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 6 - protein, 1916 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINLGGDSGGSGGSGGSVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQASGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 7 - protein, 1937 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVLGGDSGGSGGSGGSVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQASGGSKRTADGSEFEPKKKRKV

SEQ ID NO: 8 - protein, 297 aa, synthetic construct
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETAAALGPEDEAAASRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 9 - protein, 1937 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVLGGDSGGSGGSGGSVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETAAALGPEDEAAASRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQASGGSKRTADGSEFEPKKKRKV

SEQ ID NO: 10 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 11 - protein, 18 aa, synthetic construct
KRTADGSEFESPKKKRKV

SEQ ID NO: 12 - protein, 17 aa, synthetic construct
KRTADGSEFEPKKKRKV

SEQ ID NO: 13 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 14 - DNA, 86 bp, synthetic construct
  source 1..86: mol_type = other DNA
gtgggtgagggcactccggttggggggtcttaagttgggcatttgtgggggtccttcagaattcagacctgtgtgtctccttgctg

SEQ ID NO: 15 - DNA, 19 bp, synthetic construct
  source 1..19: mol_type = other DNA
gcccagcaaggagacacac

SEQ ID NO: 16 - RNA, 19 nt, synthetic construct
  source 1..19: mol_type = other RNA
gcccagcaaggagacacac

SEQ ID NO: 17 - DNA, 19 bp, synthetic construct
  source 1..19: mol_type = other DNA
gccctgcaaggagacacac

SEQ ID NO: 18 - DNA, 19 bp, synthetic construct
  source 1..19: mol_type = other DNA
gcccagcaaggagacacac

SEQ ID NO: 19 - DNA, 19 bp, synthetic construct
  source 1..19: mol_type = other DNA
gcccggcaaggagacacac

SEQ ID NO: 20 - DNA, 19 bp, synthetic construct
  source 1..19: mol_type = other DNA
gccccgcaaggagacacac

SEQ ID NO: 21 - DNA, 86 bp, synthetic construct
  source 1..86: mol_type = other DNA
gtgggtgagggcactccggttggggggtcttaagttgggcatttgtgggggtccttcagaattcagagccgtgtgtctccttgcat

SEQ ID NO: 22 - DNA, 20 bp, synthetic construct
  source 1..20: mol_type = other DNA
atgccatgcaaggagacaca

SEQ ID NO: 23 - RNA, 20 nt, synthetic construct
  source 1..20: mol_type = other RNA
atgccatgcaaggagacaca

SEQ ID NO: 24 - DNA, 20 bp, synthetic construct
  source 1..20: mol_type = other DNA
atgccctgcaaggagacaca

SEQ ID NO: 25 - DNA, 20 bp, synthetic construct
  source 1..20: mol_type = other DNA
atgccatgcaaggagacaca

SEQ ID NO: 26 - DNA, 20 bp, synthetic construct
  source 1..20: mol_type = other DNA
atgccgtgcaaggagacaca

SEQ ID NO: 27 - DNA, 20 bp, synthetic construct
  source 1..20: mol_type = other DNA
atgccttgcaaggagacaca

SEQ ID NO: 28 - protein, 297 aa, synthetic construct
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMSISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 29 - protein, 1937 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVLGGDSGGSGGSGGSVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMSISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQASGGSKRTADGSEFEPKKKRKV

SEQ ID NO: 30 - protein, 297 aa, synthetic construct
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMSISSQGDGACVLLRALEPLEGLETMRQLRATLRAATAARVLADRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 31 - protein, 1937 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVLGGDSGGSGGSGGSVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMSISSQGDGACVLLRALEPLEGLETMRQLRATLRAATAARVLADRELCSGPSKLCQALAINKSFDQRDLAQDEAVWLERGPLEPSEPAVVAAARVGVGHAGEWARKPLRFYVRGSPWVSVVDRVAEQDTQASGGSKRTADGSEFEPKKKRKV

SEQ ID NO: 32 - protein, 297 aa, synthetic construct
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYRMYFCMSISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 33 - protein, 1937 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVLGGDSGGSGGSGGSVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYRMYFCMSISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQASGGSKRTADGSEFEPKKKRKV

SEQ ID NO: 34 - protein, 297 aa, synthetic construct
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYRMYFCMSISSQGDGACVLLRALEPLEGLETMRQLRATLRAATAARVLADRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 35 - protein, 1937 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVLGGDSGGSGGSGGSVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYRMYFCMSISSQGDGACVLLRALEPLEGLETMRQLRATLRAATAARVLADRELCSGPSKLCQALAINKSFDQRDLAQDEAVWLERGPLEPSEPAVVAAARVGVGHAGEWARKPLRFYVRGSPWVSVVDRVAEQDTQASGGSKRTADGSEFEPKKKRKV

SEQ ID NO: 36 - protein, 713 aa, synthetic construct
MATGQDRVVALVDMDCFFVQVEQRQNPHLRNKPCAVVQYKSWKGGGIIAVSYEARAFGVTRSMWADDAKKLCPDLLLAQVRESRGKANLTKYREASVEVMEIMSRFAVIERASIDEAYVDLTSAVQERLQKLQGQPISADLLPSTYIEGLPQGPTTAEETVQKEGMRKQGLFQWLDSLQIDNLTSPDLQLTVGAVIVEEMRAAIERETGFQCSAGISHNKVLAKLACGLNKPNRQTLVSHGSVPQLFSQMPIRKIRSLGGKLGASVIEILGIEYMGELTQFTESQLQSHFGEKNGSWLYAMCRGIEHDPVKPRQLPKTIGCSKNFPGKTALATREQVQWWLLQLAQELEERLTKDRNDNDRVATQLVVSIRVQGDKRLSSLRRCCALTRYDAHKMSHDAFTVIKNCNTSGIQTEWSPPLTMLFLCATKFSASAPSSSTDITSFLSSDPSSLPKVPVTSSEAKTQGSGPAVTATKKATTSLESFFQKAAERQKVKEASLSSLTAPTQAPMSNSPSKPSLPFQTSQSTGTEPFFKQKSLLLKQKQLNNSSVSSPQQNPWSNCKALPNSLPTEYPGCVPVCEGVSKLEESSKATPAEMDLAHNSQSMHASSASKSVLEVTQKATPNPSLLAAEDQVPCEKCGSLVPVWDMPEHMDYHFALELQKSFLQPHSSNPQVVSAVSHQGKRNPKSPLACTNKRPRPEGMQTLESFFKPLTH

SEQ ID NO: 37 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDRCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 39 - RNA, 23 nt, synthetic construct
  source 1..23: mol_type = other RNA
agaaatgtgtccccagttgacac

SEQ ID NO: 40 - DNA, 20 bp, synthetic construct
  source 1..20: mol_type = other DNA
gacaaaccagaagccgctcc

SEQ ID NO: 41 - DNA, 20 bp, synthetic construct
  source 1..20: mol_type = other DNA
gttcacacccatgacgaaca

SEQ ID NO: 42 - DNA, 20 bp, synthetic construct
  source 1..20: mol_type = other DNA
gaacacaaagcatagactgc

SEQ ID NO: 43 - DNA, 20 bp, synthetic construct
  source 1..20: mol_type = other DNA
gaagaccaaggatagactgc

SEQ ID NO: 44 - DNA, 20 bp, synthetic construct
  source 1..20: mol_type = other DNA
gtggggaaggggcccccaag

SEQ ID NO: 45 - DNA, 20 bp, synthetic construct
  source 1..20: mol_type = other DNA
gagtccgagcagaagaagaa

SEQ ID NO: 46 - DNA, 20 bp, synthetic construct
  source 1..20: mol_type = other DNA
gtcatcttagtcattacctg

SEQ ID NO: 47 - DNA, 20 bp, synthetic construct
  source 1..20: mol_type = other DNA
gtcatccagtgctaccgctg

SEQ ID NO: 48 - DNA, 20 bp, synthetic construct
  source 1..20: mol_type = other DNA
atcttacaggaactccagga

SEQ ID NO: 49 - DNA, 20 bp, synthetic construct
  source 1..20: mol_type = other DNA
tgaatccaagtgtcctctga

SEQ ID NO: 50 - DNA, 20 bp, synthetic construct
  source 1..20: mol_type = other DNA
agacaccaaatcttactgga

SEQ ID NO: 51 - DNA, 20 bp, synthetic construct
  source 1..20: mol_type = other DNA
gccagcaagtgtgacagtca

SEQ ID NO: 52 - DNA, 20 bp, synthetic construct
  source 1..20: mol_type = other DNA
ctaggagatacacctccacc

SEQ ID NO: 53 - DNA, 20 bp, synthetic construct
  source 1..20: mol_type = other DNA
gtaaacaaagcatagactga

SEQ ID NO: 54 - DNA, 20 bp, synthetic construct
  source 1..20: mol_type = other DNA
gagtatgaggcatagactgc

SEQ ID NO: 55 - DNA, 20 bp, synthetic construct
  source 1..20: mol_type = other DNA
gtcaagaaagcagagactgc

SEQ ID NO: 56 - DNA, 20 bp, synthetic construct
  source 1..20: mol_type = other DNA
gagcaaagagaatagactgt

SEQ ID NO: 57 - DNA, 20 bp, synthetic construct
  source 1..20: mol_type = other DNA
gatgagataatgatgagtca

SEQ ID NO: 58 - DNA, 20 bp, synthetic construct
  source 1..20: mol_type = other DNA
ggattgacccaggccagggc

SEQ ID NO: 59 - DNA, 20 bp, synthetic construct
  source 1..20: mol_type = other DNA
taagcatagactccaggata

SEQ ID NO: 60 - DNA, 20 bp, synthetic construct
  source 1..20: mol_type = other DNA
cgggcatcagaattccctgg

SEQ ID NO: 61 - DNA, 20 bp, synthetic construct
  source 1..20: mol_type = other DNA
atgaggaaagggactagagt

SEQ ID NO: 62 - DNA, 20 bp, synthetic construct
  source 1..20: mol_type = other DNA
gctgactcagagaccctgag

SEQ ID NO: 63 - DNA, 20 bp, synthetic construct
  source 1..20: mol_type = other DNA
gaatactaagcatagactcc

SEQ ID NO: 64 - DNA, 20 bp, synthetic construct
  source 1..20: mol_type = other DNA
gaacataaagaatagaatga

SEQ ID NO: 65 - DNA, 20 bp, synthetic construct
  source 1..20: mol_type = other DNA
ggacaggcagcatagactgt

SEQ ID NO: 66 - DNA, 20 bp, synthetic construct
  source 1..20: mol_type = other DNA
atgactaagatgactgccaa

SEQ ID NO: 67 - DNA, 20 bp, synthetic construct
  source 1..20: mol_type = other DNA
gtcatacactgggctggcca

SEQ ID NO: 68 - DNA, 20 bp, synthetic construct
  source 1..20: mol_type = other DNA
ggtcatacactgggctggcc

SEQ ID NO: 69 - DNA, 20 bp, synthetic construct
  source 1..20: mol_type = other DNA
ctatattacttaccttatcc

SEQ ID NO: 70 - DNA, 20 bp, synthetic construct
  source 1..20: mol_type = other DNA
gaagatagagaatagactgc

SEQ ID NO: 71 - DNA, 20 bp, synthetic construct
  source 1..20: mol_type = other DNA
ggctaaagaccatagactgt

SEQ ID NO: 72 - DNA, 20 bp, synthetic construct
  source 1..20: mol_type = other DNA
gggaataaatcatagaatcc

SEQ ID NO: 73 - DNA, 20 bp, synthetic construct
  source 1..20: mol_type = other DNA
gacaaagaggaagagagacg

SEQ ID NO: 74 - DNA, 20 bp, synthetic construct
  source 1..20: mol_type = other DNA
acgtgccaggtggagcaccc

SEQ ID NO: 75 - DNA, 20 bp, synthetic construct
  source 1..20: mol_type = other DNA
catgactcagccatctgtta

SEQ ID NO: 76 - DNA, 20 bp, synthetic construct
  source 1..20: mol_type = other DNA
tagatacaaagatggtagga

SEQ ID NO: 77 - DNA, 20 bp, synthetic construct
  source 1..20: mol_type = other DNA
taattcaaaccagcagagtc

SEQ ID NO: 78 - DNA, 20 bp, synthetic construct
  source 1..20: mol_type = other DNA
tggatcaatagacaggataa

SEQ ID NO: 79 - DNA, 20 bp, synthetic construct
  source 1..20: mol_type = other DNA
ggtgggatggggtccccaag

SEQ ID NO: 80 - DNA, 20 bp, synthetic construct
  source 1..20: mol_type = other DNA
ggtagggagaggcccccaga

SEQ ID NO: 81 - DNA, 20 bp, synthetic construct
  source 1..20: mol_type = other DNA
ggtggggagcggccccccag

SEQ ID NO: 82 - DNA, 20 bp, synthetic construct
  source 1..20: mol_type = other DNA
gagttagagcagaagaagaa

SEQ ID NO: 83 - DNA, 20 bp, synthetic construct
  source 1..20: mol_type = other DNA
gagtctaagcagaagaagaa

SEQ ID NO: 84 - DNA, 20 bp, synthetic construct
  source 1..20: mol_type = other DNA
gaggccgagcagaagaaaga

SEQ ID NO: 85 - DNA, 22 bp, synthetic construct
  source 1..22: mol_type = other DNA
gtggtagacagcatgtgtccta

SEQ ID NO: 86 - DNA, 22 bp, synthetic construct
  source 1..22: mol_type = other DNA
atttacagcctggcctttgggg

SEQ ID NO: 87 - DNA, 22 bp, synthetic construct
  source 1..22: mol_type = other DNA
gtgtcaggtaatgtgctaaaca

SEQ ID NO: 88 - DNA, 22 bp, synthetic construct
  source 1..22: mol_type = other DNA
ggtggaggagggtgcatggggt

SEQ ID NO: 89 - DNA, 20 bp, synthetic construct
  source 1..20: mol_type = other DNA
tctgcttctccagccctggc

SEQ ID NO: 90 - DNA, 86 bp, synthetic construct
  source 1..86: mol_type = other DNA
cagcaaggagacacacaggtctgaattctgaaggacccccacaaatgcccaacttaagaccccccaaccggagtgccctcacccac

SEQ ID NO: 91 - DNA, 86 bp, synthetic construct
  source 1..86: mol_type = other DNA
atgcaaggagacacacggctctgaattctgaaggacccccacaaatgcccaacttaagaccccccaaccggagtgccctcacccac

(19) United States
(12) Patent Application Publication
(10) Pub. No.: US 2025/0304940 A1
(43) Pub. Date: Oct. 2, 2025

(54) PROGRAMMABLE ADENINE BASE EDITOR AND USES THEREOF

(71) Applicant: HuidaGene Therapeutics (Singapore) Pte. Ltd., Singapore (SG)

(72) Inventor: Huawei Tong, Shanghai (CN)

(21) Appl. No.: 18/865,035

(22) PCT Filed: May 12, 2023
(86) PCT No.: PCT/CN2023/094023

§ 371 Date: Nov. 12, 2024

(30) Foreign Application Priority Data
May 13, 2022 (WO) PCT/CN2022/092759
Dec. 16, 2022 (WO) PCT/CN2022/139699

(51) Int. Cl. C12N 9/78; C12N 9/22; C12N 9/24; C12N 15/11

(52) U.S. Cl. CPC C12N 9/78; C12N 9/226; C12N 9/2497; C12N 15/11; C12Y 302/02021; C12Y 305/04002; C07K 2319/00; C12N 2310/20

(57) ABSTRACT

Provided is a programmable adenine base editor and system comprising the same and method of using the same. Also provided are MPG and mutants thereof, which can be used in the programmable adenine base editor.

### CROSS-REFERENCE TO RELATED APPLICATIONS

This application claims the benefit of and priority to PCT Patent Application No. PCT/CN2022/092759, filed on May 13, 2022, entitled “ENGINGEERED N-METHYLPURINE DNA GLYCOSYLASE (MPG) AND USES THEREOF”, and PCT Patent Application No. PCT/CN2022/139699, filed on Dec. 16, 2022, entitled “PROGRAMMABLE ADENINE TRANSVERSION BASE EDITOR AND USES THEREOF”, the entire contents of which, including any sequence listing and drawings, are incorporated herein by reference in their entireties.

### REFERENCE TO AN ELECTRONIC SEQUENCE LISTING

The disclosure contains an electronic sequence listing (“HGP025PCT.xml” created on May 11, 2023, by software “WIPO Sequence” according to WIPO Standard ST. 26), which is incorporated herein by reference in its entirety. According to WIPO Standard ST. 26, symbol “t” is used to denote both T in DNA and U in RNA (See “Table 1: List of nucleotides symbols”, the definition of symbol “t” is “thymine in DNA/uracil in RNA (t/u)”). Thus, in a sequence listing prepared according to ST. 26, wherever a sequence is an RNA, the T in the sequence shall be deemed as U.

### BACKGROUND

Base editors are promising tools for precise base editing in basic research and therapeutic applications^{1,2}. Adenine base editors (ABEs) and cytosine base editors (CBEs) enable A:T to G: C and C: G to T: Atransitions, respectively^{3,4}. Recently, C-to-G base editors (CGBEs) were developed by replacing uracil glycosylase inhibitor (UGI) with uracil DNA N-glycosylase (UNG) in cytosine base editors⁵⁻⁹. However, no editor exists that can enable base conversions including transition and transversion. Base editor enabling A-to-T and A-to-C transversions remains to be achieved to repair a large number of point mutations², accounting for up to 27% genetic diseases (FIG. 3). It is needed in the art for an adenine base editor with expanded editing outcome to facilitate, for example, A-to-C and A-to-T transversion editing (AYBE, Y=C or T). especially in mammalian cells.

Citation or identification of any document in the disclosure is not an admission that such a document is available as prior art to the disclosure. Each of the references mentioned or cited in the disclosure is incorporated by reference in its entirety.

### SUMMARY

It is against the above background that the disclosure provides certain advantages and advancements over the prior art. Although the disclosure is not limited to specific advantages or functionalities, in one aspect, the disclosure provides a base editor comprising:
(1) a nucleic acid programmable DNA binding domain (napDNAbd) capable of binding a target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
(2) an adenine deaminase domain capable of deaminating the adenine of the target deoxyadenosine to a hypoxanthine; and
(3) a hypoxanthine excising domain capable of excising the hypoxanthine.

In another aspect, the disclosure provides a system comprising:
(1) a base editor or a polynucleotide encoding the base editor, the base editor comprising:
(a) a nucleic acid programmable DNA binding domain (napDNAbd) capable of binding a target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
(b) an adenine deaminase domain capable of deaminating the adenine of the target deoxyadenosine to a hypoxanthine; and
(c) a hypoxanthine excising domain capable of excising the hypoxanthine; and
(2) a guide nucleic acid or a polynucleotide encoding the guide nucleic acid, the guide nucleic acid comprising:
(a) a scaffold sequence capable of forming a complex with the napDNAbd; and
(b) a guide sequence capable of hybridizing to the target sequence on the target strand of the target dsDNA, thereby guiding the complex to the target dsDNA.

In yet another aspect, the disclosure provides a method of modifying a target dsDNA, comprising contacting the target dsDNA with a system,
the target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
the system comprising:
(1) a base editor or a polynucleotide encoding the base editor, the base editor comprising:
(a) a nucleic acid programmable DNA binding domain (napDNAbd) capable of binding the target dsDNA;
(b) an adenine deaminase domain capable of deaminating the adenine of the target deoxyadenosine to a hypoxanthine; and
(c) a hypoxanthine excising domain capable of excising the hypoxanthine; and
(2) a guide nucleic acid or a polynucleotide encoding the guide nucleic acid, the guide nucleic acid comprising:
(1) a scaffold sequence capable of forming a complex with the napDNAbd; and
(2) a guide sequence capable of hybridizing to the target sequence on the target strand of the target dsDNA, thereby guiding the complex to the target dsDNA.

In yet another aspect, the disclosure provides a polynucleotide encoding the base editor of the disclosure and optionally the guide nucleic acid as defined in the disclosure.

In yet another aspect, the disclosure provides a vector comprising the polynucleotide of the disclosure.

In yet another aspect, the disclosure provides a complex comprising the base editor of the disclosure and a guide nucleic acid as defined in the disclosure.

In yet another aspect, the disclosure provides a cell comprising the base editor or system of the disclosure, the polynucleotide of the disclosure, the vector of the disclosure, or the complex of the disclosure.

In yet another aspect, the disclosure provides a pharmaceutical composition comprising:
(i) the base editor or the system of the disclosure, the polynucleotide of the disclosure, the vector of the disclosure, the complex of the disclosure, or the cell of the disclosure; and
(ii) a pharmaceutically acceptable excipient.

In yet another aspect, the disclosure provides a method for treating a subject having or at a risk of developing a disease associated with a target deoxyadenosine of a target dsDNA, comprising administering to the subject (e.g., an effective amount of) the system of the disclosure, wherein the target deoxyadenosine is modified by the system, and the modification treats or prevents the disease.

In yet another aspect, the disclosure provides an MPG as defined in the disclosure.

The details of one or more embodiments of the disclosure are set forth in the description below. Other features or advantages of the disclosure will be apparent from the following drawings and detailed description of several embodiments, and also from the appended claims. It is understood that any aspect or embodiment of the disclosure can be combined with any other aspect or embodiment of the disclosure to constitute another embodiment explicitly or implicitly disclosed herein unless otherwise indicated.

### Definitions

The disclosure will be described with respect to particular embodiments, but the disclosure is not limited thereto but only by the claims. Unless defined otherwise, all technical and scientific terms used herein have the same meaning as is commonly understood by one of ordinary skill in the art to which this disclosure belongs. Terms as set forth hereinafter are generally to be understood in their common sense unless indicated otherwise.

### Overview

Nucleic acid programmable DNA binding protein (napDNAbp), for example, Cas9, Cas12, IscB, is substantially capable of binding to a target DNA (e.g., a dsDNA) as guided by a guide nucleic acid (e.g., a guide RNA) comprising a guide sequence targeting the target DNA. In some embodiments, the target DNA is eukaryotic.

Without wishing to be bound by theory, in some embodiments, the guide nucleic acid comprises a scaffold sequence responsible for forming a complex with the napDNAbp, and a guide sequence that is intentionally designed to be responsible for hybridizing to a target sequence of the target DNA, thereby guiding the complex comprising the napDNAbp and the guide nucleic acid to the target DNA.

Referring to FIG. 21, an exemplary target dsDNA is depicted to comprise a 5′ to 3′ single DNA strand and a 3′ to 5′ single DNA strand, the 5′ to 3′ single DNA strand comprises a first deoxyribonucleotide dA, and the 3′ to 5′ single DNA strand comprises a second deoxyribonucleotide dT that base pairs with the dA.

An exemplary guide nucleic acid is depicted to comprise a guide sequence and a scaffold sequence. The guide sequence is designed to hybridize to a part of the 3′ to 5′ single DNA strand, and so the guide sequence “targets” that part. And thus, the 3′ to 5′ single DNA strand is referred to as a “target strand (TS)” of the target dsDNA, while the opposite 5′ to 3′ single DNA strand is referred to as a “non-target strand (NTS)” of the target dsDNA. That part of the target strand based on which the guide sequence is designed and to which the guide sequence may hybridize is referred to as a “target sequence”, while the opposite part on the non-target strand corresponding to that part is referred to as the “protospacer sequence”, which is 100% (fully) reversely complementary to the target sequence.

Generally, a nucleic acid sequence (e.g., a DNA sequence, an RNA sequence) is written in 5′ to 3′ direction/orientation.

For example, for a DNA sequence of ATGC, it is usually understood as 5′-ATGC-3′ unless otherwise indicated. Its reverse sequence is 5′-CGTA-3′, its fully complement sequence is 5′-TACG-3′, and its fully reverse complement sequence is 5′-GCAT-3′.

Generally, the double-strand sequence of a dsDNA may be represented with the sequence of its 5′ to 3′ single DNA strand conventionally written in 5′ to 3′ direction/orientation unless otherwise indicated.

For example, for a dsDNA having a 5′ to 3′ single DNA strand of 5′-ATGC-3′ and a 3′ to 5′ single DNA strand of 3′-TACG-5′, the dsDNA may be simply represented as 5′-ATGC-3′.

5′-----ATGC-----3′
3′-----TACG-----5′

It should be noted that either the 5′ to 3′ single DNA strand or the 3′ to 5′ single DNA strand of a dsDNA can be a nontarget strand from which a protospacer sequence is selected.

In the sense of base editing, the strand on which the target nucleotide to be edited is located is termed as an edited strand, and the opposite strand is termed as a non-edited strand. As used herein, the nontarget strand is the edited strand, and the target strand is the non-edited strand.

Generally for a dsDNA, such as a gene, the 5′ to 3′ single DNA strand is the sense strand of the gene, and the 3′ to 5′ single DNA strand is the antisense strand of the gene. But it should be noted that either the sense strand or the antisense strand of a gene can be a nontarget strand from which a protospacer sequence is selected.

To hybridize to a target dsDNA, in one embodiment, the guide sequence of a guide nucleic acid is designed to have a sequence of 5′-AUGC-3′ that is fully reversely complementary to the 3′ to 5′ strand of the target dsDNA, which would be set forth in ATGC in the electric sequence listing but marked as an RNA sequence; and in another embodiment, the guide sequence of a guide nucleic acid is designed to have a sequence of 5′-GCAU-3′ that is fully reversely complementary to the 5′ to 3′ strand of the target dsDNA, which would be set forth in GCAT in the electric sequence listing but marked as an RNA sequence.

In the case that the guide sequence of a guide nucleic acid is fully reversely complementary to the target sequence and the target sequence is fully reversely complementary to the protospacer sequence, the guide sequence is identical to the protospacer sequence except for the U in the guide sequence due to its RNA nature and correspondingly the T in the protospacer sequence due to its DNA nature. According to WIPO standard ST. 26, symbol “t” is used to denote both T in DNA and U in RNA (See “Table 1: List of nucleotides symbols”, the definition of symbol “t” is “thymine in DNA/uracil in RNA (t/u)”). Thus, in the sequence listing of the disclosure prepared according to ST. 26, such a guide sequence could be set forth in the same sequence as a corresponding protospacer sequence. For convenience, a single SEQ ID NO in the sequence listing can be used to denote both such guide sequence and protospacer sequence, although such a single SEQ ID NO may be marked as either DNA or RNA in the sequence listing. When a reference is made to such a SEQ ID NO that sets forth a protospacer/guide sequence, it refers to either a protospacer sequence that is a DNA sequence or a guide sequence that is an RNA sequence depending on the context, no matter whether it is marked as a DNA or an RNA in the sequence listing.

### Term

Unless otherwise specified, all technical and scientific terms used in the disclosure have the meaning commonly understood by one of ordinary skill in the art to which the disclosure belongs. Throughout the specification, several terms are employed that are defined in the following paragraphs. Other definitions are also found within the body of the specification.

As used herein, the term “nucleic acid programmable DNA binding domain (napDNAbd)” may be used interchangeably with “nucleic acid programmable DNA binding protein (napDNAbp)” to refer to a protein that can associate (e.g., bind) with a programmable nucleic acid (e.g., DNA or RNA), such as a guide nucleic acid (e.g., gRNA), that is programmed to guide the protein to a specific sequence of a target DNA via the interaction (e.g., hybridization) between the programmable nucleic acid and the target DNA. The napDNAbd may be indirectly associated with (e.g., bound to) the target DNA via the interaction between the programmable nucleic acid and the target DNA.

As used herein, the terms “nucleic acid”, “nucleic acid molecule”, or “polynucleotide” are used interchangeably. They refer to a polymer of deoxyribonucleotides or ribonucleotides or their mixtures in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “polypeptide” and “protein” are used interchangeably to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component.

As used herein, a “fusion protein” refers to a protein created through the joining of two or more originally separate proteins, or portions thereof. In some embodiments, a linker may be present between each protein.

As used herein, the term “heterologous,” in reference to polypeptide domains, refers to the fact that the polypeptide domains do not naturally occur together (e.g., in the same polypeptide). For example, in fusion proteins generated by the hand of man, a polypeptide domain from one polypeptide may be fused to a polypeptide domain from a different polypeptide. The two polypeptide domains would be considered “heterologous” with respect to each other, as they do not naturally occur together.

As used herein, the term “nuclease” refers to a polypeptide capable of cleaving the phosphodiester bonds between the nucleotide subunits of nucleic acids; the term “endonuclease” refers to a polypeptide capable of cleaving the phosphodiester bond within a polynucleotide chain.

As used herein, the term “guide nucleic acid” refers to a nucleic acid-based molecule capable of forming a complex with a napDNAbp (e.g., Cas9, Cas12, IscB) (e.g., via a scaffold sequence of the guide nucleic acid), and comprises a sequence (e.g., guide sequences) that are sufficiently complementary to a target DNA to hybridize to the target DNA and guide the complex to the target DNA, which include but are not limited to RNA-based molecules, e.g., guide RNA. As used herein, the terms “crRNA”, “guide RNA (gRNA)”, “single guide RNA (sgRNA)”, and “RNA guide” are used interchangeably. As used in the disclosure, the term “guide sequence” is used interchangeably with the term “spacer sequence”, and the term “scaffold sequence” is used interchangeably with the term “direct repeat (DR) sequence”.

As used herein, the term “complex” refers to a grouping of two or more molecules. In some embodiments, the complex comprises a polypeptide and a nucleic acid interacting with (e.g., binding to, coming into contact with, adhering to) one another. As used herein, the term “complex” can refer to a grouping of a guide nucleic acid and a polypeptide (e.g., a napDNAbp). As used herein, the term “complex” can refer to a grouping of a guide nucleic acid, a polypeptide (e.g., a napDNAbp), and a target DNA.

As used herein, the term “activity” refers to a biological activity. In some embodiments, the activity includes enzymatic activity, e.g., catalytic ability of an effector. For example, the activity can include nuclease activity, e.g., DNA nuclease activity, dsDNA endonuclease activity, guide sequence-specific (on-target) dsDNA endonuclease activity, guide sequence-independent (off-target) dsDNA endonuclease activity.

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or cohesive ends.

As used herein, the meanings of “cleaving a nucleic acid” or “modifying a nucleic acid” may overlap. Modifying a nucleic acid includes not only modification of a mononucleotide but also insertion or deletion of a nucleic acid fragment.

As used herein, the term “on-target” refers to binding, cleavage, and/or editing of an intended or expected region of DNA, for example, by the base editor of the disclosure.

As used herein, the term “off-target” refers to binding, cleavage, and/or editing of an unintended or unexpected region of DNA, for example, by the base editor of the disclosure. In some embodiments, a region of DNA is an off-target region when it differs from the region of DNA intended or expected to be bound, cleaved and/or edited by 1, 2, 3, 4, 5, 6, 7, 8, 9, 10 or more nucleotides.

As used herein, if a DNA sequence, for example, 5′-ATGC-3′ is transcribed to an RNA sequence, with each dT (deoxythymidine, or “T” for short) in the primary sequence of the DNA sequence replaced with a U (uridine) and each dA (deoxyadenosine, or “A” for short), dG (deoxyguanosine, or “G” for short), and dC (deoxycytidine, or “C” for short) replaced with A (adenosine), G (guanosine), and C (cytidine), respectively, for example, 5′-AUGC-3′, it is said in the disclosure that the DNA sequence “encodes” the RNA sequence.

As used herein, the term “protospacer adjacent motif” or “PAM” refers to a short sequence (or a motif) adjacent to a protospacer sequence on the nontarget strand of a dsDNA recognized by the complex comprising a napDNAbp.

As used herein, the term “adjacent” includes instances wherein there is no nucleotide between the protospacer sequence and the PAM and also instances wherein there are a small number (e.g., 1, 2, 3, 4, or 5) of nucleotides between the protospacer sequence and the PAM. As used herein, A “immediately adjacent (to)” B, A “immediately 5′ to” B, and A “immediately 3′ to” B mean that there is no nucleotide between A and B.

As described herein, the guide sequence is so designed to be substantially capable of hybridizing to a target sequence. As used herein, the term “hybridize”, “hybridizing”, or “hybridization” refers to a reaction in which one or more polynucleotide sequences react to form a complex that is stabilized via hydrogen bonding between the bases of the one or more polynucleotide sequences. The hydrogen bonding may occur by Watson Crick base pairing, Hoogstein binding, or in any other sequence specific manner. A polynucleotide sequence capable of hybridizing to a given polynucleotide sequence is referred to as the “complement” of the given polynucleotide sequence. As used herein, the hybridization of a guide sequence and a target sequence is so stabilized to permit a napDNAbp that is complexed with a guide nucleic acid comprising the guide sequence or a function domain (e.g., a deaminase domain) associated (e.g., fused) with the napDNAbp to act (e.g., cleave, deaminize) at or near the target sequence or its complement (e.g., a sequence of a target DNA or its complement).

For the purpose of hybridization, in some embodiments, the guide sequence is reversely complementary to a target sequence. As used herein, the term “complementary” refers to the ability of nucleobases of a first polynucleotide sequence, such as a guide sequence, to base pair with nucleobases of a second polynucleotide sequence, such as a target sequence, by traditional Watson-Crick base-pairing. Two complementary polynucleotide sequences are able to non-covalently bind under appropriate temperature and solution ionic strength conditions. In some embodiments, a first polynucleotide sequence (e.g., a guide sequence) comprises 100% (fully) complementarity to a second nucleic acid (e.g., a target sequence). In some embodiments, a first polynucleotide sequence (e.g., a guide sequence) is complementary to a second polynucleotide sequence (e.g., a target sequence) if the first polynucleotide sequence comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide sequence (e.g., a guide sequence) that has a certain level of complementarity to a second polynucleotide sequence (e.g., a target sequence) such that the first polynucleotide sequence (e.g., a guide sequence) can hybridize to the second polynucleotide sequence (e.g., a target sequence) with sufficient affinity to permit a napDNAbd that is complexed with the first polynucleotide sequence or a nucleic acid comprising the first polynucleotide sequence or a function domain associated (e.g., fused) with the napDNAbd to act (e.g., cleave, deaminize) on the target sequence or its complement (e.g., a sequence of a target DNA or its complement). In some embodiments, a guide sequence that is substantially complementary to a target sequence has 100% or less than 100% complementarity to the target sequence. In some embodiments, a guide sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence.

As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. The comparison of sequences and determination of percent identity between two sequences can be accomplished using a mathematical algorithm. As is well known in the art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, the sequence identity is calculated by global alignment, for example, using the Needleman-Wunsch algorithm and an online tool at ebi.ac.uk/Tools/psa/emboss_needle/. In some embodiments, the sequence identity is calculated by local alignment, for example, using the Smith-Waterman algorithm and an online tool at ebi.ac.uk/Tools/psa/emboss_water/.

As used herein, the term “variant” refers to an entity that shows significant structural identity with a reference entity (e.g., a wild-type sequence) but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a polypeptide may have a characteristic sequence element comprising a plurality of amino acids having designated positions relative to one another in linear or three-dimensional space and/or contributing to a particular biological function; a nucleic acid may have a characteristic sequence element comprising a plurality of nucleotide residues having designated positions relative to one another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide (e.g., a nuclease described herein) that is at least 60%, 65%, 70%, 75%, 80%, 85%, 86%, 87%, 88%, 89%, 90%, 91%, 92%, 93%, 94%, 95%, 96%, 97%, 98% or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide, e.g., nuclease activity. In some embodiments, a variant polypeptide lacks one or more of the biological activities of the reference polypeptide. In some embodiments, a variant polypeptide shows a reduced level of one or more biological activities (e.g., nuclease activity, e.g., off-target nuclease activity) as compared with the reference polypeptide. In some embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2%, or 1% of the residues in the variant are substituted as compared with the parent or reference polypeptide. In some embodiments, a variant has 10, 9, 8, 7, 6, 5, 4, 3, 2, or 1 substituted residue as compared with a parent or reference polypeptide. Often, a variant has a very small number (e.g., fewer than 5, 4, 3, 2, or 1) of substituted functional residues (i.e., residues that participate in a particular biological activity). In some embodiments, a variant has not more than 5, 4, 3, 2, or 1 additions or deletions, and often has no additions or deletions, as compared with the parent or reference polypeptide. Moreover, any additions or deletions are typically fewer than about 25, about 20, about 19, about 18, about 17, about 16, about 15, about 14, about 13, about 12, about 11, about 10, about 9, about 8, about 7, about 6, and commonly are fewer than about 5, about 4, about 3, or about 2 residues. In some embodiments, the parent or reference polypeptide is a wild type. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the terms “non-naturally occurring” and “engineered” are used interchangeably and refer to artificial participation. When these terms are used to describe a nucleic acid or a polypeptide, it is meant that the nucleic acid or polypeptide is at least substantially freed from at least one other component of its association in nature or as found in nature.

Conservative substitutions of non-critical amino acids of a protein may be made without affecting the normal functions of the protein. Conservative substitutions refer to the substitution of amino acids with chemically or functionally similar amino acids. In some embodiments, a conservative amino acid substitution refers to an amino acid substitution that does not alter the relative charge or size characteristics of the protein in which the amino acid substitution was made. In some embodiments, a “conservative substitution” refers to a substitution of an amino acid made among amino acids within the following groups: i) methionine, isoleucine, leucine, valine, ii) phenylalanine, tyrosine, tryptophan, iii) lysine, arginine, histidine, iv) alanine, glycine, v) serine, threonine, vi) glutamine, asparagine and vii) glutamic acid, aspartic acid.

As used herein, the term “wild type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the description of “a variant or mutant (e.g., of a MPG) comprising an amino acid mutation (e.g., substitution) at a given position (e.g., N169) of a given polypeptide (e.g., SEQ ID NO: 2)” or similar description means that the polypeptide as set forth in the amino acid sequence of the given polypeptide serves as a parent or reference polypeptide, and the variant is a variant of the parent or reference polypeptide and comprises an amino acid mutation at a position of the amino acid sequence of the variant corresponding to the given position of the amino acid sequence of the given polypeptide. The position of the amino acid mutation in the amino acid sequence of the variant may be the same as the given position of the given polypeptide, for example, when the variant comprises just an amino acid substitution as compared with the given polypeptide and has the same length as the given polypeptide. The position of the amino acid mutation in the amino acid sequence of the variant may also be different from the given position of the given polypeptide, for example, when the variant comprises a N-terminal truncation as compared with the given polypeptide and the first N-terminal amino acid of the variant is not corresponding to the first N-terminal amino acid of the given polypeptide but to an amino acid within the given polypeptide, but the position of the amino acid mutation can be determined by alignment of the variant and the given polypeptide to identify the corresponding amino acids in their sequences as understood by a skilled in the art. For example, if the variant has a N-terminal truncation of 20 amino acids as compared with the given polypeptide, then the variant comprising an amino acid mutation at N169 of a given polypeptide means that the variant comprises an amino acid mutation at N149 of the variant since N149 in the variant is corresponding to N169 in the given polypeptide as determined by alignment of the variant and the given polypeptide, wherein the position is numbered according to the amino acid sequence of SEQ ID NO: 1.

As used herein, the description of “a variant or mutant (e.g., of a MPG) comprising a given amino acid substitution (e.g., N169S) relative to a given polypeptide (e.g., SEQ ID NO: 2)” means that the polypeptide as set forth in the amino acid sequence of the given polypeptide serves as a parent or reference polypeptide that does not comprise the given amino acid substitution, and the variant is a variant of the parent or reference polypeptide and comprises an amino acid substitution having the same type of substitution as the given amino acid substitution and at a position in the amino acid sequence of the variant corresponding to the position of the given amino acid substitution. For example, an MPG variant comprising an amino acid substitution N169S relative to SEQ ID NO: 2 refers to the fact that the amino acid sequence of SEQ ID NO: 2 comprises amino acid N at position 169, and the MPG variant comprises amino acid S at a position corresponding to N169 of the amino acid sequence of SEQ ID NO: 2, wherein the position is numbered according to the amino acid sequence of SEQ ID NO: 1. The corresponding relationship of positions in two amino acid sequences as determined by alignment is explained in the previous paragraph.

The position is numbered according to the given polypeptide as the parent or reference polypeptide. However, the position may also be numbered according to another polypeptide as the parent or reference polypeptide if specifically indicated, e.g., “wherein the position is numbered according to the amino acid sequence of SEQ ID NO: 1”. For example, SEQ ID NO: 1 is the full length wild type human MPG with N-terminal starting Methionine (M), and SEQ ID NO: 2 is a N-terminal truncation of SEQ ID NO: 1, wherein the N-terminal starting Methionine (M) is removed. The amino acid at position 169 of SEQ ID NO: 1 is N, while the corresponding amino acid N in SEQ ID NO: 2 is at position 168 of SEQ ID NO: 2. When an amino acid substitution is to be made at the amino acid N at position 168 of SEQ ID NO: 2 based on SEQ ID NO: 2 as the parent or reference polypeptide, the amino acid N can still be numbered according to SEQ ID NO: 1 and termed as N169 of SEQ ID NO: 2, although the corresponding amino acid N is indeed at position 168 of SEQ ID NO: 2, and such an amino acid substitution is termed as N169X (where X represents the amino acid that replaces N) relative to SEQ ID NO: 2.

As used herein, the terms “upstream” and “downstream” refer to relative positions within a single nucleic acid (e.g., DNA) sequence in a nucleic acid. “Upstream” and “downstream” relate to the 5′ to 3′ direction, respectively, in which transcription occurs. For a first sequence and a second sequence present on the same strand of a single nucleic acid written in 5′ to 3′ direction, the first sequence is upstream of the second sequence when the 3′ end of the first sequence is on the left side of the 5′ end of the second sequence, and the first sequence is downstream of the second sequence when the 5′ end of the first sequence is on the right side of the 3′ end of the second sequence. For example, a promoter is usually at the upstream of a sequence under the regulation of the promoter; and on the other hand, a sequence under the regulation of a promoter is usually at the downstream of the promoter.

As used herein, the term “regulatory element” refers to a DNA sequence that controls or impacts one or more aspects of transcription and/or expression is intended to include promoters, enhancers, silencers, termination signals, internal ribosome entry sites (IRES), and other expression control elements (e.g., transcription termination signals such as polyadenylation signals and poly-U sequences). Regulatory elements include those that direct constitutive expression of a nucleotide sequence in many types of host cells and those that direct expression of a nucleotide sequence only in certain host cells (e.g., tissue-specific regulatory sequences). Regulatory elements may also direct expression in a time-dependent manner, e.g., in a cell cycle-dependent or developmental stage-dependent manner, which may or may not be tissue or cell type specific.

As used herein, the term “operably linked” refers to a juxtaposition wherein the components described are in a relationship permitting them to function in their intended manner. A regulatory element “operably linked” to a functional element is associated in such a way that transcription, expression, and/or activity of the functional element is achieved under conditions compatible with the regulatory element. In some embodiments, “operably linked” regulatory elements are contiguous (e.g., covalently linked) with the functional elements of interest; in some embodiments, regulatory elements act in trans to or otherwise at a distance from the functional elements of interest.

As used herein, the term “cell” is understood to refer not only to a particular individual cell, but to the progeny or potential progeny of the cell. Because certain modifications may occur in succeeding generations due to either mutation or environmental influences, such progeny may not, in fact, be identical to the parent cell, but are still included within the scope of the term.

As used herein, the term “in vivo” means inside the body of an organism, and the terms “ex vivo” or “in vitro” means outside the body of an organism.

As used herein, the term “treat”, “treatment”, or “treating” is an approach for obtaining beneficial or desired results including clinical results. For purposes of the disclosure, the beneficial or desired clinical results include, but are not limited to, one or more of the following: alleviating one or more symptoms resulting from a disease, diminishing the extent of a disease, stabilizing a disease (e.g., preventing or delaying the worsening of a disease), preventing or delaying the spread (e.g., metastasis) of a disease, preventing or delaying the recurrence of a disease, reducing recurrence rate of a disease, delay or slowing the progression of a disease, ameliorating a disease state, providing a remission (partial or total) of a disease, decreasing the dose of one or more other medications required to treat a disease, delaying the progression of a disease, increasing the quality of life, and prolonging survival. Also encompassed by the term is a reduction of pathological consequence of a disease (such as cancer). The methods of the disclosure contemplate any one or more of these aspects of treatment.

As used herein, the term “disease” includes the terms “disorder” and “condition” and is not limited to those specific diseases that have been medically or clinically defined.

As used herein, reference to “not” a value or parameter generally means and describes “other than” a value or parameter. For example, the method is not used to treat cancer of type X means the method may be used to treat cancer of types other than X.

As used herein, the singular forms “a”, “an”, and “the” include plural referents unless the context clearly dictates otherwise. That is, articles “a/an” and “the” are used herein to refer to one or more than one (i.e., at least one) grammatical object of the article. For example, “an element” means one element or more than one element, e.g., two elements.

As used herein, the term “and/or” in a phrase such as “A and/or B” is intended to mean either or both of the alternatives, including both A and B, A or B, A (alone), and B (alone). Likewise, the term “and/or” in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, when the term “about” is ahead of a serious of numbers (for example, about 1, 2, 3), it is understood that each of the serious of numbers is modified by the term “about” (that is, about 1, about 2, about 3). The term “about X-Y” used herein has the same meaning as “about X to about Y.”

As used herein, the terms “about” and “approximately,” in reference to a number, is used herein to include numbers that fall within a range of 20%, 15%, 10%, 9%, 8%, 7%, 6%, 5%, 4%, 3%, 2%, or 1% in either direction (greater than or less than) of the number unless otherwise stated or otherwise evident from the context (except where such number would exceed 100% of a possible value).

As used herein, a numerical range includes the end values of the range, and each specific value within the range, for example, “16 to 100 nucleotides” includes 16 nucleotides and 100 nucleotides, and each specific value between 16 and 100, e.g., 17, 23, 34, 52, 78.

As used herein, the terms “comprise”, “include”, “contain”, and “have” are to be understood as implying that a stated element or a group of elements is included, but not excluding any other element or a group of elements, unless the context requires otherwise. In certain embodiments, the terms “comprise”, “include”, “contain”, and “have” are used synonymously.

As used herein, the phrase “consist essentially of” is intended to include any element listed after the phrase “consist essentially of” and is limited to other elements that do not interfere with or contribute to the activities or actions specified in the disclosure of the listed elements. Thus, the phrase “consist essentially of” is intended to indicate that the listed elements are required, but no other elements are optional, and may or may not be present depending on whether they affect the activities or actions of the listed elements.

As used herein, the phrase “consist of” means including but limited to any element after the phrase “consist of”. Thus, the phrase “consist of” indicates that the listed elements are required, and that no other elements can be present.

As used herein, the term “comprises” also encompasses the terms “consists essentially of” and “consists of”. It is understood that the “comprising” embodiments of the disclosure described herein also include “consisting essentially of” and “consisting” embodiments.

Throughout the specification, reference to “one embodiment”, “embodiment”, “a specific embodiment”, “a related embodiment”, “an embodiment”, “another embodiment”, or “a further embodiment” or a combination thereof means that specific elements, features, structures, or characteristics described in connection with the embodiment are included in at least one embodiment of the disclosure. Accordingly, the appearances of the foregoing phrases in various places throughout the specification are not necessarily all referring to the same embodiments. Furthermore, specific elements, features, structures, or characteristics may be combined in any suitable manner in one or more embodiments.

It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1 shows, in some embodiments, engineering and optimization of adenine transversion base editor. FIG. 1a, Schematic diagram of potential pathway for adenine transversion and editing outcomes. After adenine deamination on the edited (nontarget) strand by ABE8e and nicking on the nonedited (target) strand by Cas9 nickase (nCas9-D10A), MPG induces hypoxanthine excision, followed by DNA repair/translesio n synthesis (TLS) and/or replication, thus leading to diverse editing outcomes. I, deoxyinosine (the corresponding base is hypoxanthine (Hx)). MPG, N-methylpurine DNA glycosylase. AP, apurinic/apyrimidinic site. DSB, double-strand break. TLS: translesion synthesis. FIG. 1b, Schematic designs of reporter and transversion base editor constructs for A-to-T editing detection. Y=C or T. P2A, 2A peptide from porcine teschovirus-1. FIG. 1c, Representative flow cytometry scatter plots showing gating strategy and the percentages of EGFP⁺ cells for each base editor. NT, non-target. FIG. 1d, Percentage of EGFP⁺ cells. e, MFI (mean fluorescence intensity) of EGFP. Dotted line, mean value of wild-type MPG group. Fold changes are calculated relative to the wild-type MPG group. a. u., arbitrary units. n=3 in FIG. 1d and FIG. 1e. FIG. 1f, Schematic of mutagenesis and screening strategy. MPG-N169S was a constant mutation during the two rounds of mutagenesis and screening. FIG. 1g and FIG. 1h, Performance of engineered AYBE variants measured by EGFP expression in Round 1 and Round 2 mutagenesis and screening. Each dot represents the mean of three biological replicates of each AYBE variant. Dotted line, mean value of MPG-N169S group. Fold changes are calculated relative to the MPG-N169S group. FIG. 1i, Gradual improvement of AYBE-mediated EGFP activation. n=3. Dotted line, mean value of wild-type MPG group. Fold changes are calculated relative to the wild-type MPG group. All values are presented as mean±s.e.m.

FIG. 2 shows, in some embodiments, characterization of editing profiles for AYBE via high-throughput target sequencing. FIG. 2a, Bar plots showing the on-target DNA base editing frequencies of adenines with most A-to-C and/or A-to-T edits with ABE8e and AYBEv3 at the top 12 efficiently edited genomic sites in HEK293T cells. Editing frequencies of three independent replicates (n=3) at each base are displayed side-by-side. Transfected mCherry positive cells were sorted for further characterization. FIG. 2b-2d, Editing purity of A-to-C(b), A-to-T (c), or A-to-G (d) by ABE8e and AYBEv3 at the edited sites from FIG. 2a. FIG. 2e, Frequencies of A-to-C and A-to-T editing by AYBEv3 across the protospacer positions 1-20 from the edited sites in FIG. 2a (where PAM is at positions 21-23). Single dot represents individual replicate (n=3 independent replicates per site). Boxes span the interguartile range (IQR) (25th to 75th percentile), horizontal lines indicate the median (50^{th} percentile); and whiskers extend to minima and maxima. FIG. 2f, gRNA-dependent off-target (OT) analysis comparing ABE8e and AYBEv3 at site 5 (HBG) and site 6 (EMX1) (n=3). Note that a high-fidelity version¹⁷ TadA8e^{V106W} was used in ABE8e and AYBEv3. FIG. 2g, gRNA-independent off-target editing detected by the orthogonal R-loop assay at each R-loop site (n=3). FIG. 2h, Potential correction of DMD (Duchenne muscular dystrophy) nonsense mutation by AYBEv3. Allele frequencies of on-target editing by AYBEv3 in stable HEK293T cell lines generated via lentiviral transduction. Arrowheads in red indicate targeted adenines for correction. Arrowheads in green show the allele correction with potential therapeutic benefits. The values in right represent frequencies and reads of mutation alleles. FIG. 2i, Schematic diagram of potential pathway to increase the A-to-T editing outcomes. FIG. 2j-2k, A-to-T editing outcomes for the introduction of Poln (n=3). FIG. 21, Diagram showing types of achievable point mutations with the available base editors. All values are presented as mean±s.e.m.

FIG. 3 shows, in some embodiments, distribution of human pathogenic SNP variants and demonstration of potential codon transversions with adenine transversion base editor. FIG. 3a, Base pair changes required to correct pathogenic human SNPs in the ClinVar database (accessed Jul. 23, 2022). FIG. 3b, Table of all potential codon transversions enabled by A-to-C or A-to-T editing. The potential outcomes by AYBE (adenine transversion base editor) are highlighted in blue.

FIG. 4. shows, in some embodiments, prototype AYBEs and structure of MPG. FIG. 4a, prototype AYBE candidates designed in three orientations/configurations: MTC, TMC, and TCM. M=MPG, T=TadA8e, C=nCas9. FIG. 4b, Percentage of EGFP⁺ cells for each prototype. All values are presented as mean±s.e.m., n=3. FIG. 4c, View of MPG structure in ribbon representation predicted by AlphaFold (alphafold. com/entry/P29372). The non-conserved N-terminal region (1-79 aa) is intrinsically disordered, and the rest region of MPG contributes to base excision and DNA binding activities.

FIG. 5 shows, in some embodiments, characterizations of A-to-C and A-to-T reporter. FIG. 5a, Schematic construct designs for detecting A-to-C editing. FIG. 5b, Representative flow cytometry scatter plots showing gating strategy and the percentages of BFP+ and EGFP⁺ cells for each base editor at the splice acceptor site, respectively. At least 2000 BFP⁺ cells from each sample were analyzed. FIG. 5c, Representative flow cytometry scatter plots showing gating strategy and the percentages of EGFP⁺ cells for each base editor.

FIG. 6 shows, in some embodiments, gradual improvement of AYBE-mediated transversion editing at an endogenous genomic and effective residue shown on structure. FIG. 6a, Bar plots showing on-target DNA base editing frequencies with ABE8e and different AYBE variants at site 3. Editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. FIG. 6b, A-to-T and A-to-C outcomes with ABE8e and different AYBE variants at the edited sites A7 from FIG. 6a. FIG. 6c, Percentage of alleles that contain an insertion and/or deletion across the entire protospacer with various base editor from FIG. 6a. All values are presented as mean±s.e.m. Transfected mCherry positive cells were sorted for further characterization. FIG. 6d, Location of effective residues of AYBEv3 variant shown in magenta on the three-dimensional structure. Overlaid structures with DNA from a deposited structure (PDB 1BNK, shown in gray) and a structure for 78-298aa region of MPG protein predicted by AlphaFold (from FIG. 4c).

FIG. 7 shows, in some embodiments, characterization of editing profiles for AYBEv3 at the top 12 efficiently edited genomic sites in HEK293T cells. Bar plots showing on-target DNA base editing frequencies with ABE8e and AYBEv3. Editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. Arrowheads indicate adenines with the most transversion edits. Transfected mCherry positive cells were sorted for further characterization of AYBEv3.

FIG. 8 shows, in some embodiments, characterization of AYBEv3. Frequencies of A-to-G and A-to-Y by ABE8e or AYBEv3 editor across the protospacer positions 1-20 from FIG. 7 (where PAM is at positions 21-23). Data are presented as median values.

FIG. 9 shows, in some embodiments, characterization of editing profiles for AYBEv3 at seven more genomic sites with an A7 or A8. Bar plots showing on-target DNA base editing frequencies with ABE8e and AYBEv3. Editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. Arrowheads indicate adenines with the most transversion edits. Transfected mCherry positive cells were sorted for further characterization of AYBEv3.

FIG. 10 shows, in some embodiments, characterization of editing profiles for AYBEv3 at seven more genomic sites with high editing activity at A5, A6, or A9. Bar plots showing on-target DNA base editing frequencies with ABE8e and AYBEv3. Editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. Arrowheads indicate adenines with the most transversion edits. Transfected mCherry positive cells were sorted for further characterization of AYBEv3.

FIG. 11 shows, in some embodiments, additional characterization of AYBEv3 on-target editing activities in HEK293T cells. FIG. 11a, Dot and box plots representing the combined distribution of A-to-C, A-to-T, A-to-G and indel frequencies per nucleotide across the entire protospacer from experiments performed with ABE8e and AYBEv3 using 26 guide sequences. For indels frequencies, percentage of alleles that contain an insertion and/or deletion across the entire protospacer for different sites were also presented at bottom. Single dot represents individual replicate (n=3 independent replicates per site). FIG. 11b, Frequencies of A-to-C and A-to-T editing at NAN DNA motifs appear in the protospacer positions 6-9 from genomic sites with AYBEv3 used in FIG. 11a. Each dot indicates mean value of the motif from corresponding site. Boxes span the IQR (25^{th} to 75^{th} percentile), horizontal lines indicate the median (50^{th} percentile) and whiskers extend to minima and maxima. Data points in plots represent full range of values plotted. The graphs were derived from the data shown in FIGS. 7, 9, and 10.

FIG. 12 shows, in some embodiments, allele compositions following treatment with AYBEv3. FIG. 12a-12b, Allele frequencies of DNA on-target editing within site 33 (FIG. 12a) and site 35 (FIG. 12b) by AYBEv3 in HEK293T cells. The values in right represent frequencies and reads of mutation alleles. Site 33 has multiple A within the target window, some of which were edited to C while others were edited to T. AYBEv3 could induce less bystander editing than ABE8e. Decreased percentage of alleles simultaneously containing multiple edits after treatment with AYBEv3 compared to ABE8e was observed.

FIG. 13 shows, in some embodiments, characterization of editing profiles for AYBEv3 in HeLa, U2OS, and K562 cells. Bar plots showing on-target DNA base editing frequencies with ABE8e and AYBEv3 using 5 gRNAs targeting genomic sites in different cells. Editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. Arrowheads indicate adenines with the most transversion edits. Transfected mCherry positive cells were sorted for further characterization of AYBEv3.

FIG. 14 shows, in some embodiments, additional characterization of AYBEv3 on-target editing activities in HeLa, U2OS, and K562 cells. FIG. 14a, Dot and box plots representing the combined distribution of A-to-C, A-to-T and indel frequencies per nucleotide across the entire protospacer from experiments performed with AYBEv3 using 5 guide sequences. Boxes span the IQR (25th to 75th percentile), horizontal lines indicate the median (50th percentile), and whiskers extend to minima and maxima. Data points in plots represent full range of values plotted. Single dots represent individual replicates (n=3 independent replicates per site). FIG. 14b, A-to-C and A-to-T editing purity for AYBEv3 in different cells. n=3. All values are presented as mean±s.e.m. The graphs were derived from the data shown in FIG. 13.

FIG. 15 shows, in some embodiments, off-target analysis of AYBEv3. FIG. 15a, Bar plots showing on-target DNA base editing frequencies with ABE8e (TadA8e-V106W) and AYBEv3 (TadA8e-V106W) at site 5 (HBG) and site 6 (EMX1) in HEK293T cells. FIG. 15b, Orthogonal R-loop assay overview. FIG. 15c, Bar plots showing on-target DNA base editing frequencies with ABE8e and AYBEv3 at site 3. FIG. 15d, On-target base editing efficiencies for ABE8e and AYBEv3 at A5 and A7 of site 3 in HEK293T cells. Dots represent individual biological replicates and bars represent mean±s.e.m., n=3. In FIG. 15a and FIG. 15c, editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. Arrowheads indicate adenines with the most transversion edits. Transfected mCherry positive cells were sorted for further characterization of AYBEv3.

FIG. 16 shows, in some embodiments, potential correction of disease-related transversion mutations with AYBEv3. FIG. 16a-16b, DNA sequencing chromatograms of different disease-related mutations corrected by A-to-C editing (FIG. 16a) or A-to-T editing (FIG. 16b) with AYBEv3. SAS, splicing acceptor site. Arrows indicate targeted adenines for correction. The corresponding consequences of the correction were showed below. The mutation in DMD gene was associated with Duchenne muscular dystrophy. The mutation in SLC26A4 gene was associated with autosomal recessive non-syndromic hearing loss 4. The mutation in ATM gene was associated with Ataxia-telangiectasia syndrome. The mutation in TTN gene was associated with Dilated cardiomyopathy 1G. FIG. 16c, Stacked bar plot showing the on-target DNA base editing frequencies of 4 targeted disease-related mutations with AYBEv3 in stable HEK293T cell lines generated via lentiviral transduction. Editing frequencies of corrected base are displayed (n=3). All values are presented as mean±s.e.m. FIG. 16d-16f, Allele frequencies of on-target editing for different disease-related mutations corrected by AYBEv3. Arrowheads in red indicate targeted adenines for correction. Arrowheads in green show the allele correction with potential therapeutic benefits. The values in right represent frequencies and reads of mutation alleles. The data shown is representative of three biological replicates.

FIG. 17 shows, in some embodiments, characterization of editing profiles for AYBEv3 together with Poln. Bar plots showing on-target DNA base editing frequencies with AYBEv3 and AYBEv3+Polη. Editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. Arrowheads indicate adenines with the most transversion edits. Transfected mCherry positive cells were sorted for further characterization of AYBEv3.

FIG. 18 shows, in some embodiments, characterization of editing profiles for AYBE developed from ABE8e or ABEmax. Bar plots showing on-target DNA base editing frequencies with ABE8e and AYBEv3 using 10 genomic sites in HEK293T cells. Editing frequencies of three independent replicates (n=3) at each base are displayed side-by-side. The protospacer positions of target adenines are highlighted in red. Percentage values below specific adenines bases indicate the average A-to-T (light green) or A-to-C(light blue) editing observed. Arrowheads indicate adenines with the most transversion edits. Transfected mCherry positive cells were sorted for further characterization of AYBEv3.

FIG. 19 shows, in some embodiments, transversion activity of dead Cas9-containing AYBEs as compared with ABE8e.

FIG. 20 shows, in some embodiments, transversion activity of Cas12i nickase-containing AYBEs as compared with ABE8e.

FIG. 21 illustrates, in some embodiments, before base editing, an exemplify target dsDNA containing an exemplify target deoxyribonucleotide dA, an exemplify guide nucleic acid, and an exemplify napDNAbp that is a nickase (but may also not be a nickase in some other embodiments).

FIG. 22 illustrates, in some embodiments, after base editing, an exemplify target dsDNA containing an exemplify deoxyribonucleotide dC as base editing outcome.

FIG. 23 illustrates, in some embodiments, after base editing, an exemplify target dsDNA containing an exemplify deoxyribonucleotide dT as base editing outcome.

The figures herein are for illustrative purposes only and are not necessarily drawn to scale.

### DETAILED DESCRIPTION

### Overview

The disclosure provides a novel adenine base editor capable of expanding the editing outcome and system comprising the same and methods of using the same.

### BE

In an aspect, the disclosure provides a base editor comprising:
(1) a nucleic acid programmable DNA binding domain (napDNAbd) capable of binding a target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
(2) an adenine deaminase domain capable of deaminating the adenine of the target deoxyadenosine to a hypoxanthine; and
(3) a hypoxanthine excising domain capable of excising the hypoxanthine.

The base editor comprises a nucleic acid programmable DNA binding domain (napDNAbd). The napDNAbd may be associated with a guide nucleic acid (e.g., a guide RNA), which localizes/targets the napDNAbd to a target DNA that comprises a DNA strand (i.e., a target strand) that is complementary to the guide nucleic acid, or a portion thereof (e.g., the guide sequence of a guide RNA). In other words, the guide nucleic acid “programs” the napDNAbd to localize and bind to the target DNA. Binding of the napDNAbd of the base editor to the target DNA enables the functional domains of the base editor to access to and function on the target DNA as required. The components of the base editor are described more specifically in below.

### BE System

In another aspect, the disclosure provides a system comprising:
(1) a base editor or a polynucleotide encoding the base editor, the base editor comprising:
(a) a nucleic acid programmable DNA binding domain (napDNAbd) capable of binding a target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
(b) an adenine deaminase domain capable of deaminating the adenine of the target deoxyadenosine to a hypoxanthine; and
(c) a hypoxanthine excising domain capable of excising the hypoxanthine; and
(2) a guide nucleic acid or a polynucleotide encoding the guide nucleic acid, the guide nucleic acid comprising:
(a) a scaffold sequence capable of forming a complex with the napDNAbd; and
(b) a guide sequence capable of hybridizing to the target sequence on the target strand of the target dsDNA, thereby guiding the complex to the target dsDNA.

In some embodiments, the system is a complex comprising the base editor complexed with the guide nucleic acid. In some embodiments, the complex further comprises the target dsDNA hybridized with the guide sequence. In some embodiments, the system is a composition comprising the component (1) and the component (2). The components of the system are described more specifically in below. The guide nucleic acid is so designed to target the base editor comprising the napDNAbp to the target dsDNA, by relying on the hybridization between the guide sequence and the target dsDNA.

### Be Method

In yet another aspect, the disclosure provides a method of modifying a target dsDNA, comprising contacting the target dsDNA with a system,
the target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
the system comprising:
(1) a base editor or a polynucleotide encoding the base editor, the base editor comprising:
(a) a nucleic acid programmable DNA binding domain (napDNAbd) capable of binding the target dsDNA;
(b) an adenine deaminase domain capable of deaminating the adenine of the target deoxyadenosine to a hypoxanthine; and
(c) a hypoxanthine excising domain capable of excising the hypoxanthine; and
(2) a guide nucleic acid or a polynucleotide encoding the guide nucleic acid, the guide nucleic acid comprising:
(1) a scaffold sequence capable of forming a complex with the napDNAbd; and
(2) a guide sequence capable of hybridizing to the target sequence on the target strand of the target dsDNA, thereby guiding the complex to the target dsDNA.

The components of the method are described more specifically in below.

### Identity of Target Deoxyadenosine

In some embodiments, the target dsDNA is a wild type.

In some embodiments, the target deoxyadenosine is native to the target dsDNA.

In some embodiments, the target deoxyadenosine is a mutation in the target dsDNA.

In some embodiments, the target deoxyadenosine is a pathogenic mutation in the target dsDNA.

In some embodiments, the target dsDNA is a target gene.

### Mechanism

Without wishing to be bound to any particular theory, it is believed that the adenine base editing ability of the base editor of the disclosure relies on the ability of the hypoxanthine excising domain to excise the hypoxanthine induced by the deamination by the adenine deaminase domain.

In some embodiments, the target deoxyadenosine (first deoxyribonucleotide) is replaced with a fourth deoxyribonucleotide that is different from the target deoxyadenosine (first deoxyribonucleotide) by the base editor.

In some embodiments, the adenine of the target deoxyadenosine is deaminized by the adenine deaminase domain to form a hypoxanthine in situ.

In some embodiments, the hypoxanthine excising domain is substantially capable of excising the hypoxanthine formed in situ by the adenine deaminase domain.

In some embodiments, the hypoxanthine excising domain is substantially capable of cleaving or hydrolyzing the glycosidic bond linking the hypoxanthine formed in situ and the deoxyribose of the target deoxyadenosine.

In some embodiments, the excision of the hypoxanthine formed in situ converts the target deoxyadenosine in the protospacer sequence to an abasic site having the sugar-phosphate backbone of the target deoxyadenosine.

In some embodiments, the target strand is nicked by the napDNAbd.

In some embodiments, the nicking at the target strand induces a deletion in the target strand.

In some embodiments, the dsDNA is in a target cell.

In some embodiments, the deletion at the target strand is repaired, e.g., by translesion synthesis (TLS) in the target cell using the protospacer sequence containing the abasic site as a repair template.

In some embodiments, during the repair of the target strand, a third deoxyribonucleotide (e.g., dG, dA) different from the deoxythymidine (dT) (second deoxyribonucleotide) is formed at the site in the target sequence opposite to the abasic site in the protospacer sequence as a repair template.

In some embodiments, the sugar-phosphate backbone of the target deoxyadenosine at the abasic site is removed, e.g., by an enzyme in the target cell.

In some embodiments, upon the removal of the sugar-phosphate backbone of the target deoxyadenosine at the abasic site, a fourth deoxyribonucleotide (e.g., dC, dT) is formed at the abasic site in the protospacer sequence to base pair with the third deoxyribonucleotide (e.g., dG, dA) in the target sequence, leading to replacement of a target deoxyadenosine to a fourth deoxyribonucleotide (e.g., dA-to-dC, dA-to-dT) in the protospacer sequence.

In some embodiments, the third deoxyribonucleotide is dA, dC, or dG.

In some embodiments, the fourth deoxyribonucleotide is dT, dC, or dG.

In some embodiments, the replacement of the target deoxyadenosine to the fourth deoxyribonucleotide is dA-to-dC, dA-to-dT, or dA-to-dG.

In some embodiments, the replacement converts a stop codon to a non-stop codon or converts a non-stop codon to a stop codon.

In some embodiments, the stop codon is on the sense strand of the dsDNA.

In some embodiments, the replacement occurs on the sense strand or the nonsense strand of the dsDNA.

In some embodiments, the replacement occurs on the sense strand of the dsDNA, converting a stop codon on the sense strand to a non-stop codon or converts a non-stop codon on the sense strand to a stop codon.

In some embodiments, the replacement occurs on the nonsense strand of the dsDNA, converting a stop codon on the sense strand to a non-stop codon or converts a non-stop codon on the sense strand to a stop codon.

In some embodiments, the replacement occurs in the splicing site (e.g., splicing donor, splicing acceptor) of the target dsDNA.

In some embodiments, the replacement occurring in the splicing site (e.g., splicing donor, splicing acceptor) increases or decreases the translation of a transcript transcribed from the target dsDNA.

### Be Orientation

In some embodiments, the base editor is a fusion protein.

In some embodiments, the base editor comprises, from N-terminal to C-terminal,
(1) the adenine deaminase domain, the napDNAbd, and the hypoxanthine excision domain;
(2) the hypoxanthine excision domain, the adenine deaminase domain, and the napDNAbd; or
(3). the adenine deaminase domain, the hypoxanthine excision domain, and the napDNAbd.

In some embodiments, any two adjacent domains of (1), (2), or (3) are fused with or without a linker. Suitable linkers include, for example, those listed in WO2020/181195, which is incorporated herein by reference in its entirety.

### Base Excision Domain

In some embodiments, the base editor comprises one, two, three, or more hypoxanthine excising domains.

In some embodiments, the hypoxanthine excising domain is substantially capable of or has been engineered to be substantially capable of excising the hypoxanthine.

In some embodiments, the hypoxanthine excising domain comprises a glycosylase or

a variant thereof.

In some embodiments, the glycosylase or a variant thereof is substantially capable of or has been engineered to be substantially capable of excising the hypoxanthine.

Various glycosylases are known in the art, including those listed in WO2020/181195, which is incorporated herein by reference in its entirety. Representative glycosylases include, for example, N-methylpurine DNA glycosylase (MPG), 8-oxoguanine DNA glycosylase (OGG1), methyl-CpG binding domain 4, DNA glycosylase (MBD4), thymine DNA glycosylase (TDG), uracil DNA glycosylase (UNG), single-strand-selective monofunctional uracil-DNA glycosylase 1 (SMUG1), mutY DNA glycosylase (MUTYH), nth like DNA glycosylase 1 (NTHL1), nei like DNA glycosylase 1 (NEIL1), nei like DNA glycosylase 2 (NEIL2), nei like DNA glycosylase 3 (NEIL3), and mutants or variants capable of excising the hypoxanthine.

### MPG

In some embodiments, the hypoxanthine excising domain comprises a N-methylpurine DNA glycosylase protein (MPG).

In some embodiments, the MPG is substantially capable of or has been engineered to be substantially capable of excising the hypoxanthine.

In some embodiments, the MPG substantially has or has been engineered to substantially have N-methylpurine DNA glycosylase activity.

In some embodiments, the MPG comprises a motif Gxx Yxxxx YGxxxxxN.

Non-limiting examples of the MPG include any MPG from any of the species selected from Table A. In some embodiments, the MPG is obtained from a species selected from Table A. In some embodiments, the MPG is a variant of an MPG obtained from a species selected from Table A.

Non-limiting examples of the MPG include any MPG as set forth in Table B. In some embodiments, the MPG is a variant of human MPG (SEQ ID NO: 1 or 2) or any MPG as set forth in Table B.

In some embodiments, the MPG comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 1 or 2 or any MPG as set forth in Table B.

In some embodiments, the MPG comprises an amino acid substitution at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, and/or 297 of SEQ ID NO: 2 or a corresponding position of a MPG of another species other than human (e.g., a species selected from Table A other than human), wherein the position is numbered according to the amino acid sequence of SEQ ID NO: 1.

In some embodiments, the MPG comprises an amino acid substitution at position N169 of SEQ ID NO: 2 or a corresponding position of an MPG of another species other than human (e.g., a species selected from Table A other than human), wherein the position is numbered according to the amino acid sequence of SEQ ID NO: 1.

In some embodiments, the amino acid substitution is a substitution with Alanine (Ala/A) or Serine (Ser/S).

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 28. In some embodiments, the MPG comprises the amino acid sequence of SEQ ID NO: 28.

In some embodiments, the MPG further comprises an amino acid substitution at position S198, K202, G203, S206, and/or K210 of SEQ ID NO: 28, wherein the position is numbered according to the amino acid sequence of SEQ ID NO: 1.

In some embodiments, the amino acid substitution is a substitution with Alanine (Ala/A).

In some embodiments, the MPG further comprises an amino acid substitution selected from the group consisting of S198A, K202A, G203A, S206A, and K210A relative to SEQ ID NO: 28, wherein the position is numbered according to the amino acid sequence of SEQ ID NO: 1.

In some embodiments, the MPG comprises amino acid substitutions N169S, S198A, K202A, G203A, S206A, and K210A relative to SEQ ID NO: 2, wherein the position is numbered according to the amino acid sequence of SEQ ID NO: 1.

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 30. In some embodiments, the MPG comprises the amino acid sequence of SEQ ID NO: 30.

In some embodiments, the MPG further comprises an amino acid substitution at position S78, P79, K80, G81, R110, T115, E116, R120, R138, G163, Q173, G174, D175, A177, E185, L187, E188, L190, E191, T192, Q195, S198, T199, R201, K202, V208, K210, R212, S216, K220, A226, N228, K229, S230, Q238, E240, A241, R246, L249, P251, E253, P254, A255, R272, P274, V279, R280, G281, V291, Q294, D295, T296, Q297, and/or A298 of SEQ ID NO: 28, wherein the position is numbered according to the amino acid sequence of SEQ ID NO: 1.

In some embodiments, the amino acid substitution is a substitution with Arginine (Arg/R) or Lysine (Lys/K).

In some embodiments, the MPG further comprises an amino acid substitution selected from the group consisting of S78R, P79R, K80R, G81R, R110K, T115R, E116R, R120K, R138K, G163R, Q173R, G174R, D175R, A177R, E185R, L187R, E188R, L190R, E191R, T192R, Q195R, S198R, T199R, R201K, K202R, V208R, K210R, R212K, S216R, K220R, A226R, N228R, K229R, S230R, Q238R, E240R, A241R, R246K, L249R, P251R, E253R, P254R, A255R, R272K, P274R, V279R, R280K, G281R, V291R, Q294R, D295R, T296R, Q297R, and A298R relative to SEQ ID NO: 28, wherein the position is numbered according to SEQ ID NO: 1.

In some embodiments, the MPG comprises amino acid substitutions N169S and G163R relative to SEQ ID NO: 2, wherein the position is numbered according to SEQ ID NO: 1.

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 32. In some embodiments, the MPG comprises the amino acid sequence of SEQ ID NO: 32.

In some embodiments, the MPG comprises amino acid substitutions N169S, G163R, S198A, K202A, G203A, S206A, and K210A relative to SEQ ID NO: 2, wherein the position is numbered according to the amino acid sequence of SEQ ID NO: 1.

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 34. In some embodiments, the MPG comprises the amino acid sequence of SEQ ID NO: 34.

### Adenine Deaminase Domain

Various adenine deaminases are known in the art, including, for example, those listed in WO2020/181195, which is incorporated herein by reference in its entirety. Representative adenine deaminases include, for example, TadA and homologs and variants thereof, and APOBEC and homologs and variants thereof.

In some embodiments, the adenine deaminase domain comprises a tRNA adenosine deaminase (TadA) or a functional variant or fragment thereof, e.g., TadA8e (SEQ ID NO: 3), TadA8.17, TadA8.20, TadA9, TadA8E^{V106W}, TadA8E^{V106W+D108Q} TadA-CDa, TadA-CDb, TadA-CDc, TadA-CDd, TadA-CDe, TadA-dual, T_{AD}AC-1.2, TAD AC-1.14, T_{AD}AC-1.17, T_{AD}AC-1.19, T_{AD}AC-2.5, T_{AD}AC-2.6, T_{AD}AC-2.9, T_{AD}AC-2.19, T_{AD}AC-2.23, TadA8e-N46L, TadA8e-N46P.

In some embodiments, the adenine deaminase domain comprises an apolipoprotein B mRNA-editing complex (APOBEC) family deaminase, an activation induced deaminase (AID), a cytidine deaminase 1 from Petromyzon marinus (pmCDA1), or a functional variant or fragment thereof, e.g., APOBEC1, APOBEC2, APOBEC3A, APOBEC3B, APOBEC3C, APOBEC3D, APOBEC3F, APOBEC3G, APOBEC3H.

In some embodiments, the adenine deaminase domain comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 3.

In some embodiments, the adenine deaminase domain comprises the amino acid sequence of SEQ ID NO: 3.

napDNAbd

Various napDNAbd are known in the art, including, for example, those listed in WO2020/181195, which is incorporated herein by reference in its entirety. Representative napDNAbd include, for example, CRISPR-associated (Cas) proteins, IscB, IsrB, and TnpB.

In some embodiments, the napDNAbd substantially lacks dsDNA cleavage activity.

In some embodiments, the napDNAbd substantially lacks dsDNA cleavage activity and nickase activity.

In some embodiments, the napDNAbd has nickase activity.

In some embodiments, the napDNAbd has nickase activity to nick the target strand.

In some embodiments, the napDNAbd comprises a Cas nickase or a dead Cas of a Cas protein.

In some embodiments, the Cas protein is selected from a group consisting of a Cas9 protein (such as, SpCas9, SaCas9, GeoCas9, CjCas9, Cas9-KKH, circularly permuted Cas9, Argonaute (Ago), SmacCas9, Spy-macCas9, xCas9, SpCas9-NG,); a Cas 12 protein (such as, Cas12a, AsCas12a, LbCas12a, Cas12b, Cas12c, Cas12d, Cas12e, Cas12f (Cas14), Cas12g, Cas12h, Cas12i, xCas12i, Cas12Max, hfCas12Max, Cas12j, Cas12k, Cas121, Cas12m, Cas12n, Cas120, Cas12p, Cas12q, Cas12r, Cas12s, Cas12t, Cas12u, Cas12v, Cas12w, Cas12x, Cas12y, Cas12z); a Cas13 protein (such as, Cas13a, Cas13b, Cas13c, Cas13d, Cas13e, Cas13f, Cas13x, Cas13y); Csn2; and a mutant thereof.

In some embodiments, the Cas nickase is a Cas9 nickase (nCas9), such as SpCas9 nickase (SpCas9-D10A). In some embodiments, the napDNAbd comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 4. In some embodiments, the napDNAbd comprises the amino acid sequence of SEQ ID NO: 4.

In some embodiments, the dead Cas is a dead Cas9 (dCas9), such as dead SpCas9 (SpCas9-D10A+H840A). In some embodiments, the napDNAbd comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 37. In some embodiments, the napDNAbd comprises the amino acid sequence of SEQ ID NO: 37.

In some embodiments, the Cas nickase is a Cas12i nickase (nCas12i) or dead Cas12i (dCas12i), such as a deadCas 12i of xCas12i polypeptide. In some embodiments, the napDNAbd comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 38. In some embodiments, the napDNAbd comprises the amino acid sequence of SEQ ID NO: 38.

In some embodiments, the napDNAbd comprises an IscB nickase (nIscB) or a dead IscB (dIscB) of an IscB protein (e.g., OgeuIscB).

In some embodiments, the napDNAbd comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 4, 37, or 38. In some embodiments, the napDNAbd comprise an amino acid sequence of SEQ ID NO: 4, 37, or 38.

In some embodiments, the napDNAbd comprises a TnpB nickase or a dead TnpB of a TnpB protein.

### Full Length Base Editor

In some embodiments, the base editor comprises an NLS at the N-terminal and/or C-terminal of the napDNAbp.

In some embodiments, the base editor comprises an NLS at the N-terminal and/or C-terminal of the hypoxanthine excising domain.

In some embodiments, the base editor comprises an NLS at the N-terminal and/or C-terminal of the adenine deaminase domain.

In some embodiments, the NLS is a SV40 NLS, a bpSV40 NLS (e.g., SEQ ID NO: 11 or 12), or a NP NLS (Xenopus laevis Nucleoplasmin NLS, nucleoplasmin NLS). Additional NLS suitable for the disclosure or the way of linking an NLS to any of the components of the base editor of the disclosure include, for example, those listed in WO2020/181195, which is incorporated herein by reference in its entirety.

In some embodiments, the base editor comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 5, 6, 7, 29, 31, 33, and 35. In some embodiments, the base editor comprise an amino acid sequence of any one of SEQ ID NOs: 5, 6, 7, 29, 31, 33, and 35.

Target dsDNA

In some embodiments, the target deoxyadenosine is at a position of the protospacer sequence selected from the group consisting of position 1, position 2, position 3, position 4, position 5, position 6, position 7, position 8, position 9, position 10, position 11, position 12, position 13, position 14, position 15, position 16, position 17, position 18, position 19, position 20, and a combination thereof.

In some embodiments, the target deoxyadenosine is at a position of the protospacer sequence selected from the group consisting of position 3, position 4, position 5, position 6, position 7, position 8, position 9, position 10, and a combination thereof.

In some embodiments, the target deoxyadenosine is at a position of the protospacer sequence selected from the group consisting of position 5, position 6, position 7, position 8, position 9, and a combination thereof.

In some embodiments, the target deoxyadenosine is at position 7 or 8 of the protospacer sequence.

In some embodiments, the target deoxyadenosine is the N₂ nucleotide in a motif of N₁N₂N₃, wherein N₁, N₂, or N₃ is A, T, G, or C. In some embodiments, the target deoxyadenosine is the deoxyadenosine (dA) in a motif of CAA or CAG.

### Protospacer Sequence

In some embodiments, the protospacer sequence comprises about or at least about 16 contiguous nucleotides of the target dsDNA, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target dsDNA, or in a numerical range between any two of the preceding values, e.g., from about 16 to about 50, or from about 17 to about 22 contiguous nucleotides of the target dsDNA. In some embodiments, the protospacer sequence comprises about 20 contiguous nucleotides of the target dsDNA.

In some embodiments, the protospacer sequence is immediately 5′ or 3′ to a protospacer adjacent motif (PAM) comprises sequence 5′-NN-3′, 5′-NNN-3′, 5′-NNNN-3′, 5′-NNNNN-3′, or 5′-NNNNNN-3′, wherein N is A, T, G, or C.

In some embodiments, the protospacer sequence is immediately 5′ to a protospacer adjacent motif (PAM) comprises sequence 5′-NGG-3′, wherein N is A, T, G, or C.

In some embodiments, the protospacer sequence is immediately 3′ to a protospacer adjacent motif (PAM) comprises sequence 5′-TTN-3′, wherein N is A, T, G, or C.

### Guide Sequence

In some embodiments, the guide sequence is about or at least about 16 nucleotides in length, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any two of the preceding values, e.g., in a length of from about 16 to about 50 nucleotides, or from about 17 to about 22 nucleotides. In some embodiments, the spacer sequence is about 20 nucleotides in length.

In some embodiments, (1) the guide sequence is at least about 50%, 55%, 60%, 65%, 70%, 75%, 80%, 85%, 90%, 95%, or 100% (fully), optionally about 100% (fully), reversely complementary to the target sequence; (2) the guide sequence contains no more than 5, 4, 3, 2, or 1 mismatch or contains no mismatch with the target sequence; or (3) the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence when the PAM is immediately 5′ to the protospacer sequence or at the 3′ end of the guide sequence when the PAM is immediately 3′ to the protospacer sequence.

In some embodiments, the guide sequence comprises a sequence having a sequence identity of at least about 80% (e.g., at least about 80%, 85%, 90%, 91%, 92%, 93%, 94%, 95%, 96%, 97%, 98%, 99%, or 100%) to the sequence of any one of SEQ ID NOs: 40-89; or a sequence having at most 1, 2, 3, 4, 5, 6, 7, 8, 9, or 10 nucleotide differences, whether consecutive or not, compared to the sequence of any one of SEQ ID NOs: 40-89. In some embodiments, the guide sequence comprises the polynucleotide sequence of any one of SEQ ID NOs: 40-89.

### Scaffold Sequence

For the purpose of the disclosure, the scaffold sequence is compatible with the napDNAbd of the disclosure and is capable of complexing with the napDNAbd. The scaffold sequence may be a naturally occurring scaffold sequence identified along with the napDNAbd, or a variant thereof maintaining the ability to complex with the napDNAbd. Generally, the ability to complex with the napDNAbd is maintained as long as the secondary structure of the variant is substantially identical to the secondary structure of the naturally occurring scaffold sequence. A nucleotide deletion, insertion, or substitution in the primary sequence of the scaffold sequence may not necessarily change the secondary structure of the scaffold sequence (e.g., the relative locations and/or sizes of the stems, bulges, and loops of the scaffold sequence do not significantly deviate from that of the original stems, bulges, and loops). For example, the nucleotide deletion, insertion, or substitution may be in a bulge or loop region of the scaffold sequence so that the overall symmetry of the bulge and hence the secondary structure remains largely the same. The nucleotide deletion, insertion, or substitution may also be in the stems of the scaffold sequence so that the lengths of the stems do not significantly deviate from that of the original stems (e.g., adding or deleting one base pair in each of two stems correspond to 4 total base changes).

In some embodiments, the scaffold sequence is 5′ or 3′ to the guide sequence.

In some embodiments, the scaffold sequence is compatible to the napDNAbp.

In some embodiments, the scaffold sequence has substantially the same secondary structure as the secondary structure of SEQ ID NO: 13 or 39.

In some embodiments, the scaffold sequence comprises a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of SEQ ID NO: 13 or 39. In some embodiments, the scaffold sequence comprises the polynucleotide sequence of SEQ ID NO: 13 or 39.

### Translesion Synthesis (TLS) Polymerase

In some aspects, the base editor of the disclosure may be used in combination with a translesion synthesis (TLS) polymerase for improved outcome purity. By “purity” it means the percentage/proportion of an outcome among all possible outcomes. For example, purity of dT means the percentage/proportion of dT as an outcome among all possible outcomes including, for example, dA, dT, dG, and dC. TLS polymerases may have their own inclination of incorporating various deoxyribonucleotide opposite a AP site during polymerization, as listed in Table 4. By taking advantage of such inclination, the base editing outcome may be intentionally controlled to improve outcome purity. For example, human Poln (SEQ ID NO: 36) is a TLS polymerase preferentially incorporating dA opposite AP sites. With combination use of human Poln, the base editing outcome may be adjusted toward dT, thereby increasing purity of dT.

TABLE 4
DNA polymerases for incorporating perfect base opposite AP sites.

| DNA polymerases | Perfect base opposite AP sites |
|---|---|
| Polα (alpha) | dA |
| Polδ (delta)/PCNA | dA |
| Polγ (gamma) | dA |
| Polη (eta) | dT and dA |
| Polι (iota) | dT, dG, and dA |
| Polκ (kappa) | dC and dA |
| Polθ (theta) | dA |
| REV1 | dC |

In some embodiments, the base editor or system further comprises a translesion synthesis (TLS) polymerase or a recruiting domain capable of recruiting a TLS polymerase optionally fused to the base editor, or a coding sequence thereof.

Non-limiting examples of the TLS polymerase include Pola (alpha), Polß (beta), Polò (delta) (PCNA), Poly (gamma), Poln (eta), Poli (iota), Polk (kappa), Poli (lamda), Polu (mu), Polv (nu), Pol0 (theta), and REV1.

In some embodiments, the TLS polymerase comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 36. In some embodiments, the TLS polymerase comprises the amino acid sequence of SEQ ID NO: 36.

In some embodiments, the base editor or system further comprising the translesion synthesis (TLS) polymerase or a recruiting domain capable of recruiting a TLS polymerase leads to replacement of the target deoxyadenosine (first deoxyribonucleotide) with dC, dT, or dG.

### Additional Cytidine Deaminase Domain

In some aspects, the base editor of the disclosure may be used in combination with a cytidine deaminase domain for improved outcome purity, especially the purity of dT. By “purity” it means the percentage/proportion of an outcome among all possible outcomes. For example, purity of dT means the percentage/proportion of dC as an outcome among all possible outcomes including, for example, dA, dT, dG, and dC. It is believed that the introduction of cytidine deaminase domain may contribute to further conversion of outcome dC to dT by C-to-T base editing. So in summary there is a two-stage conversion, first, the target dA is converted to dC by the A-to-C base editing as described herein, and second, the dC is converted to dT by the C-to-T base editing.

In some embodiments, the base editor or system further comprises a cytidine deaminase domain.

In some embodiments, the base editor or system further comprising the cytidine deaminase domain leads to replacement of the target deoxyadenosine (first deoxyribonucleotide) with dT.

In some embodiments, the cytidine deaminase domain facilitates the conversion of the fourth deoxyribonucleotide that is dC to dT.

### Extended

In yet another aspect, the disclosure provides a polynucleotide encoding the base editor of the disclosure and optionally the guide nucleic acid as defined in the disclosure.

In yet another aspect, the disclosure provides a vector comprising the polynucleotide of the disclosure.

In yet another aspect, the disclosure provides a complex comprising the base editor of the disclosure and a guide nucleic acid as defined in the disclosure.

In yet another aspect, the disclosure provides a cell comprising the base editor or system of the disclosure, the polynucleotide of the disclosure, the vector of the disclosure, or the complex of the disclosure.

In yet another aspect, the disclosure provides a pharmaceutical composition comprising:
(i) the base editor or the system of the disclosure, the polynucleotide of the disclosure, the vector of the disclosure, the complex of the disclosure, or the cell of the disclosure; and
(ii) a pharmaceutically acceptable excipient.

In yet another aspect, the disclosure provides a method for treating a subject having or at a risk of developing a disease associated with a target deoxyadenosine of a target dsDNA, comprising administering to the subject (e.g., an effective amount of) the system of the disclosure, wherein the target deoxyadenosine is modified by the system, and the modification treats or prevents the disease.

### MPG

In yet another aspect, the disclosure provides an MPG substantially capable of or has been engineered to be substantially capable of excising hypoxanthine.

In some embodiments, the MPG is not wild type human MPG (hMPG; SEQ ID NO: 1), hMPG-N169A, hMPG-N169S, hMPG-N169D, hMPG-N169H, or a variant thereof without N-terminal starting Methionine (M) (e.g., SEQ ID NO: 2).

In some embodiments, the MPG substantially has or has been engineered to substantially have N-methylpurine DNA glycosylase activity.

In some embodiments, the MPG comprises a motif Gxx Yxxxx YGxxxxxN.

In some embodiments, the MPG is obtained from a species selected from Table A.

In some embodiments, the MPG is a variant of an MPG obtained from a species selected from Table A.

In some embodiments, the MPG is a variant of human MPG (SEQ ID NO: 1 or 2) or any MPG as set forth in Table B.

In some embodiments, the MPG comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 1 or 2 or any MPG as set forth in Table B.

In some embodiments, the MPG comprises an amino acid substitution at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, and/or 297 of SEQ ID NO: 2 or a corresponding position of a MPG of another species other than human (e.g., a species selected from Table A other than human), wherein the position is numbered according to the amino acid sequence of SEQ ID NO: 1.

In some embodiments, the MPG comprises an amino acid substitution at position N169 of SEQ ID NO: 2 or a corresponding position of an MPG of another species other than human (e.g., a species selected from Table A other than human), wherein the position is numbered according to the amino acid sequence of SEQ ID NO: 1.

In some embodiments, the amino acid substitution is a substitution with Alanine (Ala/A) or Serine (Ser/S).

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 28. In some embodiments, the MPG comprises the amino acid sequence of SEQ ID NO: 28.

In some embodiments, the MPG further comprises an amino acid substitution at position S198, K202, G203, S206, and/or K210 of SEQ ID NO: 28, wherein the position is numbered according to the amino acid sequence of SEQ ID NO: 1.

In some embodiments, the amino acid substitution is a substitution with Alanine (Ala/A).

In some embodiments, the MPG further comprises an amino acid substitution selected from the group consisting of S198A, K202A, G203A, S206A, and K210A relative to SEQ ID NO: 28, wherein the position is numbered according to the amino acid sequence of SEQ ID NO: 1.

In some embodiments, the MPG comprises amino acid substitutions N169S, S198A, K202A, G203A, S206A, and K210A relative to SEQ ID NO: 2, wherein the position is numbered according to the amino acid sequence of SEQ ID NO: 1.

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 30. In some embodiments, the MPG comprises the amino acid sequence of SEQ ID NO: 30.

In some embodiments, the MPG further comprises an amino acid substitution at position S78, P79, K80, G81, R110, T115, E116, R120, R138, G163, Q173, G174, D175, A177, E185, L187, E188, L190, E191, T192, Q195, S198, T199, R201, K202, V208, K210, R212, S216, K220, A226, N228, K229, S230, Q238, E240, A241, R246, L249, P251, E253, P254, A255, R272, P274, V279, R280, G281, V291, Q294, D295, T296, Q297, and/or A298 of SEQ ID NO: 28, wherein the position is numbered according to the amino acid sequence of SEQ ID NO: 1.

In some embodiments, the amino acid substitution is a substitution with Arginine (Arg/R) or Lysine (Lys/K).

In some embodiments, the MPG further comprises an amino acid substitution selected from the group consisting of S78R, P79R, K80R, G81R, R110K, T115R, E116R, R120K, R138K, G163R, Q173R, G174R, D175R, A177R, E185R, L187R, E188R, L190R, E191R, T192R, Q195R, S198R, T199R, R201K, K202R, V208R, K210R, R212K, S216R, K220R, A226R, N228R, K229R, S230R, Q238R, E240R, A241R, R246K, L249R, P251R, E253R, P254R, A255R, R272K, P274R, V279R, R280K, G281R, V291R, Q294R, D295R, T296R, Q297R, and A298R relative to SEQ ID NO: 28, wherein the position is numbered according to SEQ ID NO: 1.

In some embodiments, the MPG comprises amino acid substitutions N169S and G163R relative to SEQ ID NO: 2, wherein the position is numbered according to SEQ ID NO: 1.

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 32. In some embodiments, the MPG comprises the amino acid sequence of SEQ ID NO: 32.

In some embodiments, the MPG comprises amino acid substitutions N169S, G163R, S198A, K202A, G203A, S206A, and K210A relative to SEQ ID NO: 2, wherein the position is numbered according to the amino acid sequence of SEQ ID NO: 1.

In some embodiments, the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 34. In some embodiments, the MPG comprises the amino acid sequence of SEQ ID NO: 34.

### Regulation of Guide Nucleic Acid

In some embodiments, the polynucleotide encoding the guide nucleic acid is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the guide nucleic acid is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

### Regulation of Base Editor

In some embodiments, the polynucleotide encoding the base editor is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the polynucleotide encoding the base editor is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1a-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a human synapsin (hSyn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nß2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a OTOF promoter, a GRK1 promoter, a CRX promoter, a NRL promoter, a MECP2 promoter, a mMECP2 promoter, a hMECP2 promoter, an APP promoter, and a RCVRN promoter.

### Delivery

Various ways of delivery can be applied to the base editor of the disclosure or the system of the disclosure as needed in practices.

In yet another aspect, the disclosure provides a delivery system comprising (1) the base editor of the disclosure, the polynucleotide of the disclosure, or the system of the disclosure; and (2) a delivery vehicle.

In yet another aspect, the disclosure provides a vector comprising the polynucleotide of the disclosure. In some embodiments, the vector encodes a guide nucleic acid of the disclosure. In some embodiments, the vector is a plasmid vector, a recombinant AAV (rAAV) vector (vector genome), or a recombinant lentivirus vector.

In yet another aspect, the disclosure provides a recombinant AAV (rAAV) particle comprising the rAAV vector genome of the disclosure. A simple introduction of AAV for delivery may refer to “Adeno-associated Virus (AAV) Guide” (addgene. org/guides/aav/).

Adeno-associated virus (AAV), when engineered to delivery, e.g., a protein-encoding sequence of interest, may be termed as a (r) AAV vector, a (r) AAV vector particle, or a (r) AAV particle, where “r” stands for “recombinant”. And the genome packaged in AAV vectors for delivery may be termed as a (r) AAV vector genome, vector genome, or vg for short, while viral genome may refer to the original viral genome of natural AAVs.

The serotypes of the capsids of rAAV particles can be matched to the types of target cells. For example, Table 2 of WO2018002719A1 lists exemplary cell types that can be transduced by the indicated AAV serotypes (incorporated herein by reference).

In some embodiments, the rAAV particle comprising a capsid with a serotype suitable for delivery into ear cells (e.g., inner hair cells). In some embodiments, the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV. PHP. eB, a member of the Clade to which any of the AAV1-AAV13 belong, or a functional variant (e.g., a functional truncation) thereof, encapsidating the rAAV vector genome. In some embodiments, the serotype of the capsid is AAV9 or a functional variant thereof.

General principles of rAAV particle production are known in the art. In some embodiments, rAAV particles may be produced using the triple transfection method (described in detail in U.S. Pat. No. 6,001,650).

The vector titers are usually expressed as vector genomes per ml (vg/ml). In some embodiments, the vector titer is above 1×10⁹, above 5×10¹⁰, above 1×10¹¹, above 5×10¹¹, above 1×10¹², above 5×10¹², or above 1×10¹³ vg/ml.

Instead of packaging a single strand (ss) DNA sequence as a vector genome of a rAAV particle, systems and methods of packaging an RNA sequence as a vector genome into a rAAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of rAAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be a functional RNA sequence for use, or an RNA sequence can be produced from the RNA coding sequence, e.g., by RNA processing, or a protein can be translated from the RNA coding sequence.

For example, a base editor coding sequence encoding a base editor covers either a base editor DNA coding sequence from which a base editor is expressed (indirectly via transcription and translation) or a base editor RNA coding sequence from which a base editor is translated (directly).

For example, a gRNA coding sequence encoding a gRNA covers either a gRNA DNA coding sequence from which a gRNA is transcribed or a gRNA RNA coding sequence (1) which per se is the functional gRNA for use, or (2) from which a gRNA is produced, e.g., by RNA processing.

In some embodiments for rAAV RNA vector genomes, 5′-ITR and/or 3′-ITR as DNA packaging signals may be unnecessary and can be omitted at least partly, while RNA packaging signals can be introduced.

In some embodiments for rAAV RNA vector genomes, a promoter to drive transcription of DNA sequences may be unnecessary and can be omitted at least partly.

In some embodiments for rAAV RNA vector genomes, a sequence encoding a polyA signal may be unnecessary and can be omitted at least partly, while a polyA tail can be introduced.

Similarly, other DNA elements of rAAV DNA vector genomes can be either omitted or replaced with corresponding RNA elements and/or additional RNA elements can be introduced, in order to adapt to the strategy of delivering an RNA vector genome by rAAV particles.

In yet another aspect, the disclosure provides a ribonucleoprotein (RNP) comprising the base editor of the disclosure and a guide nucleic acid of the disclosure.

In yet another aspect, the disclosure provides a lipid nanoparticle (LNP) comprising an RNA (e.g., mRNA) encoding the base editor of the disclosure and a guide nucleic acid of the disclosure.

### Method of Modification

The system of the disclosure comprising the base editor of the disclosure has a wide variety of utilities, including modifying (e.g., cleaving, deleting, inserting, translocating, inactivating, or activating) a target DNA in a multiplicity of cell types. The systems have a broad spectrum of applications requiring high cleavage activity and small sizes, e.g., drug screening, disease diagnosis and prognosis, and treating various genetic disorders.

The methods and/or the systems of the disclosure can be used to modify a target DNA, for example, to modify the translation and/or transcription of one or more genes of the cells. For example, the modification may lead to increased transcription/translation/expression of a gene. In other embodiments, the modification may lead to decreased transcription/translation/expression of a gene.

In yet another aspect, the disclosure provides a method for modifying a target DNA, comprising contacting the target DNA with the system of the disclosure, the vector of the disclosure, the ribonucleoprotein of the disclosure, or the lipid nanoparticle of the disclosure, wherein the guide sequence is capable of hybridizing to a target sequence of the target DNA, wherein the target DNA is modified by the complex.

In some embodiments, the target DNA is in a cell.

In some embodiments, the modification comprises one or more of cleavage, base editing, repairing, and exogenous sequence insertion or integration of the target DNA.

### Cells

The methods of the disclosure can be used to introduce the systems of the disclosure into a cell and cause the cell to alter the production of one or more cellular produces, such as antibody, starch, ethanol, or any other desired products. Such cells and progenies thereof are within the scope of the disclosure.

In yet another aspect, the disclosure provides a cell comprising the system of the disclosure. In some embodiments, the cell is a eukaryote. In some embodiments, the cell is a human cell.

In yet another aspect, the disclosure provides a cell modified by the system of the disclosure or the method of the disclosure. In some embodiments, the cell is a eukaryote. In some embodiments, the cell is a human cell. In some embodiments, the cell is modified in vitro, in vivo, or ex vivo.

In some embodiments, the cell is a stem cell. In some embodiments, the cell is not a human embryonic stem cell. In some embodiments, the cell is not a human germ cell.

In some embodiments, the cell is a prokaryotic cell.

In some embodiments, the cell is a eukaryotic cell (e.g., an animal cell, a vertebrate cell, a mammalian cell, a non-human mammalian cell, a non-human primate cell, a rodent (e.g., mouse or rat) cell, a human cell, a plant cell, or a yeast cell) or a prokaryotic cell (e.g., a bacteria cell).

In some embodiments, the cell is from a plant or an animal.

In some embodiments, the plant is a dicotyledon. In some embodiments, the dicotyledon is selected from the group consisting of soybean, cabbage (e.g., Chinese cabbage), rapeseed, brassica, watermelon, melon, potato, tomato, tobacco, eggplant, pepper, cucumber, cotton, alfalfa, eggplant, grape.

In some embodiments, the plant is a monocotyledon. In some embodiments, the monocotyledon is selected from the group consisting of rice, corn, wheat, barley, oat, sorghum, millet, grasses, Poaceae, Zizania, Avena, Coix, Hordeum, Oryza, Panicum (e.g., Panicum miliaceum), Secale, Setaria (e.g., Setaria italica), Sorghum, Triticum, Zea, Cymbopogon, Saccharum (e.g., Saccharum officinarum), Phyllostachys, Dendrocalamus, Bambusa, Yushania.

In some embodiments, the animal is selected from the group consisting of pig, ox, sheep, goat, mouse, rat, alpaca, monkey, rabbit, chicken, duck, goose, fish (e.g., zebra fish).

In some embodiments, the cell is a eukaryotic cell, such as a mammalian cell, including a human cell (a primary human cell or an established human cell line). In some embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc.). In some embodiments, the cell is from fish (such as salmon), bird (such as poultry bird, including chick, duck, goose), reptile, shellfish (e.g., oyster, claim, lobster, shrimp), insect, worm, yeast, etc. In some embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

### Pharmaceutical Composition

In yet another aspect, the disclosure provides a pharmaceutical composition comprising (1) the system of the disclosure, the vector of the disclosure, the ribonucleoprotein of the disclosure, the lipid nanoparticle of the disclosure, or the cell of the disclosure; and (2) a pharmaceutically acceptable excipient.

In some embodiments, the pharmaceutical composition comprises the rAAV particle in a concentration selected from the group consisting of about 1×10¹⁰ vg/mL, 2×10¹⁰ vg/mL, 3×10¹⁰ vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical range between any of two preceding values, e.g., in a concentration of from about 9×10¹⁰ vg/mL to about 8×10¹¹ vg/mL.

In some embodiments, the pharmaceutical composition is an injection.

In some embodiments, the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

### Method of Treatment

In yet another aspect, the disclosure provides a method for diagnosing, preventing, or treating a disease in a subject in need thereof, comprising administering to the subject (e.g., a therapeutically effective dose of) the system of the disclosure, the vector of the disclosure, the ribonucleoprotein of the disclosure, the lipid nanoparticle of the disclosure, the cell of the disclosure, or the pharmaceutical composition of the disclosure, wherein the disease is associated with a target DNA, wherein the guide sequence is capable of hybridizing to a target sequence of the target DNA, wherein the target DNA is modified by the complex, and wherein the modification of the target DNA diagnose, prevents, or treats the disease.

In some embodiments, the disease is selected from the group consisting of Angelman syndrome (AS), Alzheimer's disease (AD), transthyretin amyloidosis (ATTR), transthyretin amyloid cardiomyopathy (ATTR-CM), cystic fibrosis (CF), hereditary angioedema, diabetes, progressive pseudohypertrophic muscular dystrophy, Duchenne muscular dystrophy (DMD), Becker muscular dystrophy (BMD), spinal muscular atrophy (SMA), alpha-1-antitrypsin deficiency, Pompe disease, myotonic dystrophy, Huntington's disease (HTT), fragile X syndrome, Friedreich ataxia, amyotrophic lateral sclerosis (ALS), frontotemporal dementia, hereditary chronic kidney disease, hyperlipidemia, Leber congenital amaurosis (LCA), sickle cell disease, thalassemia (e.g., β-thalassemia), Parkinson's disease (PD), myelodysplastic syndrome (MDS), retinitis pigmentosa (RP), age-related macular degeneration (AMD), Hepatitis B, nonalcoholic fatty liver disease (NAFLD), Acquired Immune Deficiency Syndrome, corneal dystrophy (CD), hypercholesterolemia, familial hypercholesterolemia (FH), heart disease (e.g., hypertrophic cardiomyopathy (HCM)), and cancer.

In some embodiments, the target DNA encodes a mRNA, a tRNA, a ribosomal RNA (rRNA), a microRNA (miRNA), a non-coding RNA, a long non-coding (Inc) RNA, a nuclear RNA, an interfering RNA (iRNA), a small interfering RNA (siRNA), a ribozyme, a riboswitch, a satellite RNA, a microswitch, a microzyme, or a viral RNA.

In some embodiments, the target DNA is a eukaryotic DNA.

In some embodiments, the eukaryotic DNA is a mammal DNA, such as a non-human mammalian DNA, a non-human primate DNA, a human DNA, a plant DNA, an insect DNA, a bird DNA, a reptile DNA, a rodent (e.g., mouse, rat) DNA, a fish DNA, a nematode DNA, or a yeast DNA.

In some embodiments, the target DNA is in a eukaryotic cell, for example, a human cell, a non-human primate cell, or a mouse cell.

In some embodiments, the administrating comprises local administration or systemic administration.

In some embodiments, the administrating comprises intrathecal administration, intramuscular administration, intravenous administration, transdermal administration, intranasal administration, oral administration, mucosal administration, intraperitoneal administration, intracranial administration, intracerebroventricular administration, or stereotaxic administration.

In some embodiments, the administration is injection or infusion.

In some embodiments, the subject is a human, a non-human primate, or a mouse.

In some embodiments, the level of the transcript (e.g., mRNA) of the target DNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target DNA in the subject prior to the administration.

In some embodiments, the level of the transcript (e.g., mRNA) of the target DNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target DNA in the subject prior to the administration.

In some embodiments, the level of the expression product (e.g., protein) of the target DNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target DNA in the subject prior to the administration.

In some embodiments, the level of the expression product (e.g., protein) of the target DNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target DNA in the subject prior to the administration. In some embodiments, the expression product is a functional mutant of the expression product of the target DNA.

In some embodiments, the median survival of the subject suffering from the disease but receiving the administration is 5 days, 10 days, 20 days, 30 days, 2 months, 3 months, 4 months, 5 months, 6 months, 7 months, 8 months, 9 months, 10 months, 11 months, 12 months, 1.5 year, 2 years, 2.5 years, 3 years, 4 years, 5 years, 6 years, 7 years, 8 years, 9 years, 10 years or more longer than that of a subject or a population of subjects suffering from the disease and not receiving the administration.

The therapeutically effective dose may be either via a single dose, or multiple doses. One skilled in the art understands that the actual dose may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

For example, the therapeutically effective dose of the rAAV particle may be about 1.0E+8, 2.0E+8, 3.0E+8, 4.0E+8, 6.0E+8, 8.0E+8, 1.0E+9, 2.0E+9, 3.0E+9, 4.0E+9, 6.0E+9, 8.0E+9, 1.0E+10, 2.0E+10, 3.0E+10, 4.0E+10, 6.0E+10, 8.0E+10, 1.0E+11, 2.0E+11, 3.0E+11, 4.0E+11, 6.0E+11, 8.0E+11, 1.0E+12, 2.0E+12, 3.0E+12, 4.0E+12, 6.0E+12, 8.0E+12, 1.0E+13, 2.0E+13, 3.0E+13, 4.0E+13, 6.0E+13, 8.0E+13, 1.0E+14, 2.0E+14, 3.0E+14, 4.0E+14, 6.0E+14, 8.0E+14, 1.0E+15, 2.0E+15, 3.0E+15, 4.0E+15, 6.0E+15, 8.0E+15, 1.0E+16, 2.0E+16, 3.0E+16, 4.0E+16, 6.0E+16, 8.0E+16, or 1.0E+17 vg, or within a range of any two of the those point values. vg stands for vector genomes of rAAV particles for administration.

### Method of Detection

In yet another aspect, the disclosure provides a method of detecting a target DNA, comprising contacting the target DNA with the system of the disclosure, wherein the target DNA is modified by the complex, and wherein the modification detects the target DNA. In some embodiments, the modification generates a detectable signal, e.g., a fluorescent signal.

### Kits

In yet another aspect, the disclosure provides a kit comprising the base editor of the disclosure, the system of the disclosure, the polynucleotide of the disclosure, the vector of the disclosure, the RNP of the disclosure, the LNP of the disclosure, the delivery system of the disclosure, the cell of the disclosure, or the pharmaceutical composition of the disclosure, or any one, two, or all components of the same.

In some embodiments, the kit further comprises an instruction to use the component(s) contained therein, and/or instructions for combining with additional component(s) that may be available or necessary elsewhere.

In some embodiments, the kit further comprises one or more buffers that may be used to dissolve any of the component(s) contained therein, and/or to provide suitable reaction conditions for one or more of the component(s). Such buffers may include one or more of PBS, HEPES, Tris, MOPS, Na₂CO₃, NaHCO₃, NaB, or combinations thereof. In some embodiments, the reaction condition includes a proper pH, such as a basic pH. In some embodiments, the pH is between 7-10.

In some embodiments, any one or more of the kit components may be stored in a suitable container or at a suitable temperature, e.g., 4 Celsius degree.

Further embodiments are illustrated in the following Examples which are given for illustrative purposes only and are not intended to limit the scope of the disclosure.

### Examples

### Material and Methods

Unless otherwise specified, the experimental methods used in the Examples are conventional.

Unless otherwise specified, the materials, reagents, etc., used in the Examples are commercially available.

Unless otherwise specified, the following materials and experimental methods were used in the Examples.

### Molecular Cloning

Base editor constructs used in this study were cloned into a mammalian expression plasmid backbone under the control of an EFla promoter by standard molecular cloning techniques. KOD-Plus-Neo DNA polymerase (KOD-401, TOYOBO) was used to amplify the insertion fragments, and NEBuilder HiFi DNA Assembly Master Mix (E2621L, New England Biolabs) was used to perform the Gibson assembly of multiple DNA fragments. The Gibson reaction was then transformed into chemically competent E. Coli. DH5a.

The wild-type human MPG sequence without N-terminal starting Methionine (M) (297 amino acids long, SEQ ID NO: 2) was PCR-amplified from cDNA of HEK293T and fused to ABESe at three different orientations with respect to nCas9 (D10A) via the Gibson assembly method. Thus, bpNLS-^{M}PG-Linker-^{T}adA8e-Linker-n^{C}as9 (D10A)-bpNLS (“MTC”), bpNLS-^{T}adA8e-Linker-^{M}PG-Linker-n^{C}as9 (D10A)-bpNLS (“TMC”), and bpNLS-^{T}adA8e-Linker-n^{C}as9 (D10A)-bpNLS-^{M}PG-bpNLS (“TCM”) fusion proteins were generated as prototype versions of adenine transversion base editor (AYBE) for A-to-T and A-to-C editing. MPG-N₁₆₉S mutation was introduced via site-directed mutagenesis by PCR.

For disease-related single nucleotide variant (SNV) transversion editing, four disease-related mutations with the upstream and downstream flanking sequences (50 bp) were constructed in tandem into lentivirus vector. The human Poln sequence was PCR-amplified from cDNA of HEK293T. bpNLS-Polη-P2A-BFP driven by a CAG promoter was constructed by standard molecular cloning techniques.

### Design and Construct of MPG Mutants

N169S was a constant mutation during the two rounds of MPG mutant screening in Example 2 based on human MGP-N169S mutant without N-terminal starting Methionine (M) (SEQ ID NO: 3). MPG mutagenesis libraries were designed and generated as previously described¹⁶. The amino acid sequence from position 78 to position 298 (numbering based on human wild type MPG with N-terminal starting Methionine as set forth in SEQ ID NO: 1) of MPG-N169S mutant was divided into 13 segments, with each 17 aa long. Thirteen (13) BpiI-harboring mutants were introduced via site-directed mutagenesis by PCR. In Round 1 screening, 52 mutants were designed, with 4 or 5 random mutation sites distributed near-uniformly in distance for each variant. All non-alanine amino acids were replaced with alanine (X>A). To cover all the residues in the segments mentioned herein, we also mutated alanine to valine (A>V). In Round 2 screening, 221 mutants scanning the protein with sequential arginine (X>R) substitutions were designed, with all arginine amino acids replaced with lysine to cover all the residues in the segments mentioned here, because both have similar size and charge. Oligos coding for mutants in the two rounds of screening were annealed and ligated into corresponding BpiI-digested backbone vectors. The MPG mutants were listed in Table 2 and 3.

### Cell Culture, Transfection, and Flow Cytometry Analysis

HEK293T, Hela, and U2OS cells were cultured with DMEM (11995065, Gibco) supplemented with 10% fetal bovine serum (04-001-1ACS, BI Worldwide) and 0.1 mM non-essential amino acids (11875-093, Gibco). K562 cells were cultured with RPMI-1640 (11875-093, Gibco) supplemented with 10% fetal bovine serum (04-001-1ACS, BI Worldwide), 1% penicillin-streptomycin (15070-063, Gibco), and 0. mM non-essential amino acids (11140-050, Gibco). Cells were grown in an incubator at 37° C. with 5% CO₂.

MPG mutant screening was conducted in 48-well plates or 24-well plates. The day before transfection, 3×10⁴ HEK293T cells per well were plated in 250 μL of complete growth medium in the 48-well plates. After 12 h, 100 ng AYBE plasmids and 200 ng A-to-T reporter plasmids were co-transfected into cells with 600 ng polyethylenimine (PEI) (DNA/PEI ratio of 1:2.5) per well. In the 24-well plates, 2×10⁵ cells were plated per well in 500 μL of complete growth medium, and 150 ng AYBE plasmids and 300 ng reporter plasmids were co-transfected into HEK293T cells with 900 ng PEI.

Disease-related SNV transversion editing was tested in stable HEK293T cell lines via lentiviral. For lentivirus packaging, plasmid with disease-related mutations (1.2 μg) was co-transfected with the packaging plasmids Pax2 (0.9 μg) and Vsvg (0.6 μg) into HEK293T cells using the FuGENE HD transfection reagent (E2311, Promega). After 72 h lentivirus-containing media was collected for infection and then filtered through a 0.45-μm low protein binding membrane (Millipore). For lentiviral infection, HEK293T cells were dissociated by trypsin-EDTA (25200-072, Gibco), and suspensions were diluted to 18×10⁵ cells per well in 6-well plates, and incubated with 150 μl lentiviruses for 48 h. Then, the medium was replaced with fresh complete medium.

For cell transfection of HEK293T, Hela, U2OS, and K562 cells for FACS, 5×10⁵ cells per well were plated in 12-well plates with 1 ml complete growth medium the day before transfection. After 14-16 h, 2 μg AYBE-gRNA plasmids were transfected into cells using PEI (DNA/PEI ratio of 1:2.5) or FuGENE HD transfection reagent (E2311, Promega) (DNA: FuGENE ratio of 1:3).

Orthogonal R-loop assays were performed as described previously¹⁷, with minor modifications. Then, 1 μg of AYBE plasmid with single guide RNA (sgRNA) targeting site 3 and 1 μg of dSaCas9 plasmid with corresponding sgRNA targeting five OT sites to generate R-loops were co-transfected into HEK293T cells in 12-well plates using PEI (DNA: PEI ratio of 1:2.5).

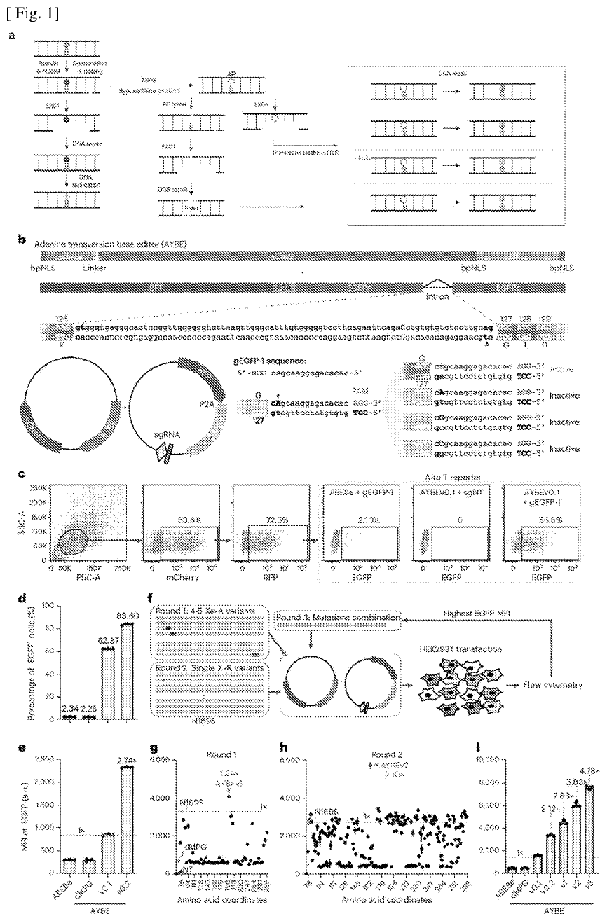

48 h after transfection, expression of mCherry, BFP, and EGFP fluorescence was analyzed by BD FACSAria III or Beckman CytoFLEX S. Flow cytometry results were analyzed with FlowJo version 10.5.3. The gating strategy in the identification of mCherry+, BFP+ and EGFP+ cells for on-target editing efficiency evaluation is supplied in FIG. 1c.

### Target Sequencing of Endogenous Sites

At 72 h after transfection, 10,000 mCherry positive cells were isolated by FACS. Genomic DNA was extracted by the addition of 40 μl of lysis buffer and 1 uL proteinase K (PD101-01, Vazyme) directly into each tube of sorted cells. The genomic DNA/lysis buffer mixture was incubated at 55° C. for 45 min, followed by a 95° C. enzyme inactivation step for 10 min. The regions of interest for target sites were amplified by PCR using site-specific primers. The PCR reaction was performed at 95° C. for 5 min, 28 cycles at 95° C. for 15 s, 60° C. for 15 s, 72° C. 30 s, and a final extension at 72° C. for 5 min using Phanta Max Super-Fidelity DNA Polymerase (P505-d3, Vazyme). PCR products were purified using universal DNA purification kit (TIANGEN) according to the manufacturer's instructions and analyzed by Sanger sequencing (GENEWIZ). The amplicons were ligated to adapters and sequencing was performed on the Illumina MiSeq platform. Protospacer sequences/guide sequences (SEQ ID NOs: 40-89) for the tested genomic locus are listed in Table 1.

TABLE 1

| | Protospacer sequence/ | SEQ ID | |
|---|---|---|---|
| site# | guide (spacer) sequence | NO | PAM |
| On-targets | | | |
| site1 (CTNNB1) | GACAAACCAGAAGCCGCTCC | 40 | TGG |
| site2 (GAPDH) | GTTCACACCCATGACGAACA | 41 | TGG |
| site3 (VISTA enhancer) | GAACACAAAGCATAGACTGC | 42 | GGG |
| site4 (HIRA) | GAAGACCAAGGATAGACTGC | 43 | TGG |
| site5 (HGB2) | GTGGGGAAGGGGCCCCCAAG | 44 | AGG |
| site6 (EXM1) | GAGTCCGAGCAGAAGAAGAA | 45 | GGG |
| site7 (RNF2) | GTCATCTTAGTCATTACCTG | 46 | AGG |
| site8 (ABLIM3) | GTCATCCAGTGCTACCGCTG | 47 | TGG |
| site9 (DMD) | ATCTTACAGGAACTCCAGGA | 48 | TGG |
| site10 (TTR) | TGAATCCAAGTGTCCTCTGA | 49 | TGG |
| sitell (TTR) | AGACACCAAATCTTACTGGA | 50 | AGG |
| site12 (PCSK9) | GCCAGCAAGTGTGACAGTCA | 51 | TGG |
| site13 (PCSK9) | CTAGGAGATACACCTCCACC | 52 | AGG |
| site14 (PSMB2) | GTAAACAAAGCATAGACTGA | 53 | GGG |
| site15 | GAGTATGAGGCATAGACTGC | 54 | AGG |
| site16 (CHM13) | GTCAAGAAAGCAGAGACTGC | 55 | CGG |
| site17 (CHM13) | GAGCAAAGAGAATAGACTGT | 56 | AGG |
| site18 (CIIM13) | GATGAGATAATGATGAGTCA | 57 | GGG |
| site19 (LINC01509) | GGATTGACCCAGGCCAGGGC | 58 | TGG |
| site20 (UPS46) | TAAGCATAGACTCCAGGATA | 59 | AGG |
| site21 (NIBAN1) | CGGGCATCAGAATTCCCTGG | 60 | AGG |
| site22 (PSMB2) | ATGAGGAAAGGGACTAGAGT | 61 | AGG |
| site23 (PPP1R12C) | GCTGACTCAGAGACCCTGAG | 62 | TGG |
| site24 | GAATACTAAGCATAGACTCC | 63 | AGG |
| site25 | GAACATAAAGAATAGAATGA | 64 | TGG |
| site26 (SEMA6D) | GGACAGGCAGCATAGACTGT | 65 | GGG |
| site27 (RNF2) | ATGACTAAGATGACTGCCAA | 66 | GGG |
| site28 (PPP1R12C) | GTCATACACTGGGCTGGCCA | 67 | GGG |
| site29 (PPP1R12C) | GGTCATACACTGGGCTGGCC | 68 | AGG |
| site30 | CTATATTACTTACCTTATCC | 69 | TGG |
| site31 (CHM13) | GAAGATAGAGAATAGACTGC | 70 | TGG |
| site32 | GGCTAAAGACCATAGACTGT | 71 | GGG |
| site33 | GGGAATAAATCATAGAATCC | 72 | TGG |
| site34 | GACAAAGAGGAAGAGAGACG | 73 | GGG |
| site35 (HFE) | ACGTGCCAGGTGGAGCACCC | 74 | AGG |
| site36 (DMD) | CATGACTCAGCCATCTGTTA | 75 | GGG |
| site37 (ATM) | TAGATACAAAGATGGTAGGA | 76 | GGG |
| site38 (SLC26A4) | TAATTCAAACCAGCAGAGTC | 77 | AGG |
| site39 (TTN) | TGGATCAATAGACAGGATAA | 78 | TGG |
| Off-targets | | | |
| SG5-OT1 | GGTGGGATGGGGTCCCCAAG | 79 | TGG |
| SG5-OT2 | GGTAGGGAGAGGCCCCCAGA | 80 | GGG |
| SG5-OT3 | GGTGGGGAGCGGCCCCCCAG | 81 | TGG |
| SG6-OT1 | GAGTTAGAGCAGAAGAAGAA | 82 | AGG |
| SG6-OT2 | GAGTCTAAGCAGAAGAAGAA | 83 | GAG |
| SG6-OT3 | GAGGCCGAGCAGAAGAAAGA | 84 | CGG |
| R-loop 1 | GTGGTAGACAGCATGTGTCCTA | 85 | AAGGGT |
| R-loop 2 | ATTTACAGCCTGGCCTTTGGGG | 86 | TCGGGT |
| R-loop 3 | GTGTCAGGTAATGTGCTAAACA | 87 | GAGAGT |
| R-loop 4 | GGTGGAGGAGGGTGCATGGGGT | 88 | CAGAAT |
| R-loop 5 | TCTGCTTCTCCAGCCCTGGC | 89 | CTGGGT |

### Target Sequencing Data Analysis.

Targeted amplicon sequencing reads were first input to trim_galore (powered by Cutadapt 0.6.6) for quality trimming, and the reads with fewer than 30 bp were filtered. The cleaned pairs were then merged using FLASH version 1.2.11. The amplified sequences from individual targets were demultiplexed using fastx_barcode_splitter. pl from the fastx_toolkit (0.0.14). Further amplicon sequencing analysis was performed by CRISPResso2²⁰. A 10-bp window was used to quantify modifications centered around the middle of the 20-bp gRNA. Otherwise, the default parameters were used for analysis. The output files, “Quantification_window_nucleotide_frequency_table. txt” and “Quantification_window_modification_count_vectors. txt” were combined to calculate the base substitution and indel rates for each individual targeting. Briefly, counts of nucleotide bases (A, C, G, and T) as well as deletion (−) and ambiguous bases (N) for each position in sgRNA were extracted from “alleles_frequency_table_ar ound_sgRNA_*. txt”. The aligned sequences with inserted bases were assigned to the reference base when insertions appear for some specific position. To give a global view of the modifications of individual position of the reference, the counts of the insertions from “Quantification_window_modification_count_vectors. txt” were introduced and used to verify the counts of the reference base though subtracting the insertion counts from the counts of reference base. The verified counts of the nucleotide bases (A, C, G, and T) as well as indels were further used to calculate the base substitution and indel rates for each position of sgRNA.

### Statistical Analysis

Statistical tests performed by GraphPad Prism 8 included the two-tailed, unpaired, two-sample t-test or Dunnett's multiple comparisons test after one-way ANOVA. All values are reported as mean+s.e.m.

### Example 1 Evaluation of Transversion Activity of Prototype AYBEs

### Prototype ABYE Construction

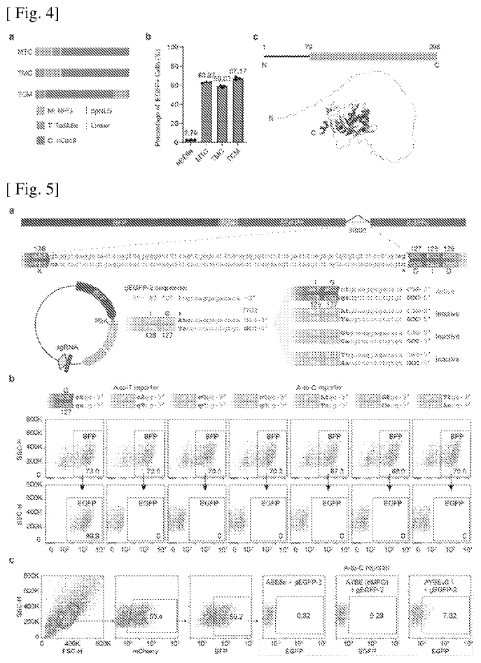

Without wishing to be bound by any particular theory, it was hypothesized by the applicant that, to induce A-to-T and A-to-C transversion editing, the excision of hypoxanthine of ABE-induced deoxyinosine might enable more versatile base editing outcomes, by triggering base excision repair (BER) pathway^{10,11} in cells (FIG. 1a). Three prototypes (MTC, TMC, and TCM) of adenine transversion base editor (AYBE, Y=C or T base) were developed by fusing ABE8e (SEQ ID NO: 3) to wild-type human N-methylpurine DNA glycosylase protein (MPG; also known as alkyladenine DNA glycosylase (AAG)) (without N-terminal starting Methionine (M); SEQ ID NO: 2), which could excise hypoxanthine (Hx) in damaged DNA^{12, 13}, at different orientations with respect to nCas9 (D10A) (SEQ ID NO: 4) (FIG. 1b and FIG. 4a).

The full-length amino acid sequence of wild type human MPG is set forth in SEQ ID NO: 1 with N-terminal starting Methionine (M) (corresponding to start codon ATG), on which the numbering of the position of any mutation of the wild type human MPG throughout the disclosure is based. In case that the wild type human MPG (or a mutant thereof) is N-terminally fused with an additional element such as a NLS or TadA, the N-terminal starting Methionine (M) (corresponding to start codon ATG) of the wild type human MGP of SEQ ID NO: 1 (or a mutant thereof) would be removed, leading to wild type human MGP without N-terminal starting Methionine (M) as set forth in SEQ ID NO: 2 (or a mutant thereof), which is termed as “MPG” (SEQ ID NO: 2) for short hereinafter unless otherwise indicated.

The amino acid sequence of TadA8e (without N-terminal starting Methionine (M) for use in AYBE is set forth in SEQ ID NO: 3.

The amino acid sequence of the nCas9 (SpCas9-D10A nickase, “nCas9” for short hereinafter unless otherwise indicated) for use in AYBE (without N-terminal starting Methionine (M), while the position of the D10A mutation is numbered based on the full length SpCas9 with N-terminal starting Methionine (M)) is set forth in SEQ ID NO: 4.

The prototype version MTC (SEQ ID NO: 5) has a configuration of N′-MPG-TadA8e-nCas9-C′. Specifically, the prototype version MTC is composed of, from N-terminal to C-terminal, a Methionine (M; corresponding to start codon ATG), bpNLS 1 (SEQ ID NO: 11), MPG (SEQ ID NO: 2), TadA8e (SEQ ID NO: 3), SpCas9-D10A nickase (SEQ ID NO: 4), and bpNLS 2 (SEQ ID NO: 12).

The prototype version TMC (SEQ ID NO: 6) has a configuration of N′-TadA8e-MPG-nCas9-C′. Specifically, the prototype version TMC is composed of, from N-terminal to C-terminal, a Methionine (M; corresponding to start codon ATG), bpNLS 1 (SEQ ID NO: 11), TadA8e (SEQ ID NO: 3), MPG (SEQ ID NO: 2), SpCas9-D10A nickase (SEQ ID NO: 4), and bpNLS 2 (SEQ ID NO: 12).

The prototype version TCM (SEQ ID NO: 7) has a configuration of N′-TadA8e-nCas9-MPG-C′. Specifically, the prototype version TCM (also termed as “AYBEv0.1”) is composed of, from N-terminal to C-terminal, a Methionine (M; corresponding to start codon ATG), bpNLS 1 (SEQ ID NO: 11), TadA8e (SEQ ID NO: 3), SpCas9-D10A nickase (SEQ ID NO: 4), bpNLS 2 (SEQ ID NO: 12), MPG (SEQ ID NO: 2), and bpNLS 2 (SEQ ID NO: 12).

In addition, a MPG mutant substantially lacking glycosylase activity (SEQ ID NO: 8; dead MPG, dMPG, or inactivated MPG) was constructed by introducing E125A, Y127A, H136A triple mutations into MPG (SEQ ID NO: 2).

Based on dMPG, AYBE-dMPG (SEQ ID NO: 9) was constructed as a negative control, which is composed of, from N-terminal to C-terminal, a Methionine (M; corresponding to start codon ATG), bpNLS 1 (SEQ ID NO: 11), TadA8e (SEQ ID NO: 3), SpCas9-D10A nickase (SEQ ID NO: 4), bpNLS 2 (SEQ ID NO: 12), dMPG (SEQ ID NO: 8), and bpNLS 2 (SEQ ID NO: 12).

In addition, ABE8e (TadA8e-nCas9 adenine base editor; SEQ ID NO: 10), which is composed of, from N-terminal to C-terminal, a Methionine (M; corresponding to start codon ATG), bpNLS 1 (SEQ ID NO: 11), TadA8e (SEQ ID NO: 3), SpCas9-D10A nickase (SEQ ID NO: 4), bpNLS 2 (SEQ ID NO: 12), was used as a blank control.

### Plasmid Construction

To conveniently evaluate the transversion activity of AYBE, a simple intron-split EGFP reporter system (FIG. 1b and FIG. 5a) comprising an expression plasmid and a reporter plasmid was designed.

The expression plasmid (vector) (FIG. 1b) comprises, in 5′-3′ orientation, a polynucleotide sequence encoding a base editor of the disclosure (e.g., MTC, TMC, TCM (also termed as “AYBEv0.1”), AYBE-dMPG (negative control), or ABE8e (blank), as described above) under the regulation of a human EF-1α promoter and followed by a sequence encoding SV40 polyA signal, and a mCherry reporter system (a polynucleotide sequence encoding mCherry under the regulation of a CBH promoter and followed by a sequence encoding bGH polyA signal) indicative of successful transfection and expression of the expression plasmid.

The reporter plasmid (vector) comprises, in 5′-3′ orientation, a polynucleotide sequence encoding, from N-terminal to C-terminal, BFP-P2A-activable EGxxFP under the regulation of a human CAG promoter and followed by a sequence encoding SV40 polyA signal, and a polynucleotide sequence encoding a EGxxFP-targeting single guide RNA (sgRNA) consisting of a EGxxFP-targeting spacer sequence and a Cas9 scaffold sequence (SEQ ID NO: 13) under the regulation of a human U6 promoter.

Seq ID NO: 13, gRNA Scaffold for Cas9, 76 bp

gttttagagctagaaatagcaagttaaaataaggctagtccgttatcaacttgaaaaagtggcaccgagtcggtgc

The intron-split EGFP reporters were engineered by insertion of the last intron (86 bp long) of human ribosomal protein S5 (RPS5) between the K126 and G127 codons of the EGFP coding sequence. Modification of the 68th base (G-to-C) or the 70th base (T-to-C) in the intron sequence for introducing artificial Cas9 protospacer adjacent motif (PAM) on the template strand, and corresponding mutations at the splice acceptor site, were made to construct A-to-T reporter or A-to-C reporter via site-directed mutagenesis by PCR, respectively. Mutations at the splice acceptor site led to inactive EGFP production by non-spliced EGFP transcripts. Transversion corrections in A-to-T reporter or A-to-C reporter were required for proper splicing of the EGxxFP coding sequence. Correctly spliced EGxxFP transcript could produce active EGFP signals detectable by flow cytometry.

In the case that the reporter plasmid was intended for A-to-T transversion evaluation, the EGFP coding sequence was inserted with a A-to-T insertion sequence of SEQ ID NO: 14 (target strand) between codon AAG (amino acid residue K at position 126) and codon GGC (amino acid residue G at position 127) of the EGFP coding sequence.

SEQ ID NO: 14, A-to-T reporter, A-to-T insertion sequence (target strand),

5′-gtgggtgagggcactccggttggggggtcttaagttgggcatttgt
gggggtccttcagaattcaga [symbol] gtgtgtctccttgcTg-3′

which also refers to its reversely complementary sequence on the non-target strand of the reporter plasmid (SEQ ID NO: 90)

(SEQ ID NO: 90)
5′-cAgcaaggagacacac [symbol] tctgaattctgaaggacccccacaaat
gcccaacttaagaccccccaaccggagtgccctcacccac -3′

The EGxxFP protospacer sequence immediately 5′ to the SpCas9 PAM ( [symbol] ) in the insertion sequence of the A-to-T reporter plasmid for designing the spacer sequence of the sgRNA is set forth in SEQ ID NO: 15,

SEQ ID NO: 15
5′-GCCcAgcaaggagacacac-3′, 19 bp,

wherein the double-underlined sequence of the protospacer sequence is a part of the insertion sequence (SEQ ID NO: 90), the upper letters GCC correspond to the codon GGC (amino acid residue G at position 127) of the EGFP coding sequence, and the double-underlined upper letter A corresponds to the double-underlined upper letter T of the insertion sequence, which indicates the target A on the reporter plasmid that is intended for A-to-T transversion.

The EGxxFP-targeting spacer sequence of the EGxxFP-targeting sgRNA for A-to-T transversion is set forth in SEQ ID NO: 16,

5′-GCC^{cAgcaaggagacacac}-3′, 19 bp, SEQ ID NO: 16 (T stands for U in the context of an RNA sequence)

which is capable of hybridizing to the reversely complementary sequence of the protospacer sequence (target sequence) on the target strand and is identical to the protospacer sequence in sequence on the nontarget strand despite of natural difference between a DNA sequence and a corresponding RNA sequence.

Four base editing consequences may be generated by use of the EGxxFP-targeting sgRNA for A-to-T transversion, as shown in FIG. 1b, where the protospacer sequence was edited to be:

SEQ ID NO: 17
(1) 5′-GCCcTgcaaggagacacac-3′,;
SEQ ID NO: 18
(2) 5′-GCCcAgcaaggagacacac-3′,;
SEQ ID NO: 19
(3) 5′-GCCcGgcaaggagacacac-3′,;
or
SEQ ID NO: 20
(4) 5′-GCCcCgcaaggagacacac-3′,.

For (1), the inserted intron can be properly spliced, leading to expression of EGFP and hence green fluorescence signals. For (2) to (4), the inserted intron cannot be properly spliced, leading to none or little expression of EGFP and hence green fluorescence signals.

A similar design was applied to A-to-C transversion evaluation (FIG. 5), where the insertion sequence was set forth in SEQ ID NO: 21 (target strand).

SEQ ID NO: 21, A-to-C reporter, A-to C insertion sequence

5′-gtgggtgagggcactccggttggggggtcttaagttgggcatttgtg
ggggtccttcagaattcagag [symbol] tgtgtctccttgcaT-3′

which also refers to its reversely complementary sequence on the non-target strand of the reporter plasmid (SEQ ID NO: 91)

(SEQ ID NO: 91)
5′- Atgcaaggagacaca [symbol] ctctgaattctgaaggacccccacaaat
gcccaacttaagaccccccaaccggagtgccctcacccac -3′

The EGxxFP protospacer sequence immediately 5′ to SpCas9 PAM ( [symbol] ) in the insertion sequence of the A-to-C reporter plasmid for designing the spacer sequence of the sgRNA is set forth in SEQ ID NO: 22,

SEQ ID NO: 22
5′-ATGCCAtgcaaggagacaca-3′, 20 bp,

wherein the double-underlined sequence of the protospacer sequence is a part of the insertion sequence (SEQ ID NO: 91), the upper letters ATGCC correspond to the codon GGC (amino acid residue G at position 127) and partial codon AT (of codon ATC; amino acid residue I at position 128) of the EGFP coding sequence, and the double-underlined upper letter A corresponds to the double-underlined upper letter T of the insertion sequence, which indicates the target A on the reporter plasmid that is intended for A-to-C transversion.

The EGxxFP-targeting spacer sequence of the EGxxFP-targeting sgRNA for A-to-C transversion is set forth in SEQ ID NO: 23,

5′-ATGCC^{Atgcaaggagacaca}-3′, 20 bp, SEQ ID NO: 23 (T stands for U in the context of an RNA sequence)

which is capable of hybridizing to the reversely complementary sequence of the protospacer sequence (target sequence) on the target strand and is identical to the protospacer sequence on the nontarget strand in sequence despite of natural difference between a DNA sequence and a corresponding RNA sequence.

Four base editing consequences may be generated by use of the EGxxFP-targeting sgRNA for A-to-C transversion, as shown in FIG. 5, where the protospacer sequence was edited to be:

SEQ ID NO: 24
(1) 5′-ATGCCCtgcaaggagacaca-3′,;
SEQ ID NO: 25
(2) 5′-ATGCCAtgcaaggagacaca-3′,;
SEQ ID NO: 26
(3) 5′-ATGCCGtgcaaggagacaca-3′,;
or
SEQ ID NO: 27
(4) 5′-ATGCCTtgcaaggagacaca-3′,.

For (1), the inserted intron can be properly spliced, leading to expression of EGFP and green fluorescence signals. For (2) to (4), the inserted intron cannot be properly spliced, leading to none or little expression of EGFP and green fluorescence signals.

### Results

Three prototype AYBEs (TCM, MTC, and TMC) were evaluated for their A-to-T transversion by using the A-to-T reporter system. After co-transfected with the A-to-T reporter vector, in which the sgRNA targeted the intronic mis-splicing mutation, the prototype TCM with MPG fused at the C-terminus (TCM, hereafter designated as AYBEv0.1) showed the highest transversion activity (67.17%) in HEK293T cells compared with the prototype MTC with MPG fused at the N-terminus (63.27%) and the prototype TMC with MPG internally fused between TadA8e and nCas9 (59.03%) (FIG. 4b). Therefore, AYBEv0.1 was used in the subsequent experiments.

Further, both the A-to-T transversion and A-to-C transversion of AYBEv0.1 were evaluated by using the A-to-T and A-to-C reporter systems, respectively. It was shown that AYBEv0.1 achieved 56.6% A-to-T transversion, whereas ABE8e without MPG (blank) and AYBEv0.1 with a non-targeting spacer sequence (sgNT, negative control) achieved 2.10% and 0% A-to-T transversion, respectively. (FIG. 1c). In additional experiments, AYBEv0.1 achieved 62.37% A-to-T transversion, whereas ABE8e without MPG (blank) and AYBE with dead MPG (negative control) achieved 2.34% and 2.25% A-to-T transversion, respectively (FIG. 1d).

On the other hand, AYBEv0.1 achieved 7.32% A-to-C transversion, whereas ABE8e without MPG (blank) and AYBE with dead MPG (negative control) achieved 0.32% and 0.29% A-to-T transversion, respectively (FIG. 5b).

It is therefore demonstrated that AYBEv0.1 could achieve significant A-to-T and A-to-C transversion compared with blank control and negative control.

Example 2 Protein engineering of MPG to improve transversion activity of AYBEv0.1

To improve the transversion activity of AYBEv0.1, rational mutagenesis of MPG was performed to generate hundreds of AYBE variants for screening, by using the A-to-T reporter to evaluate their transversion editing activity.

First, N169S, a mutation enhancing the hypoxanthine excision activity of MPG¹⁴, was introduced into the MPG domain of AYBEv0.1, thus generating AYBEv0.2 (SEQ ID NO: 29) containing MPG-N169S (SEQ ID NO: 28). It was observed that AYBEv0.2 increased the percentage (up to 83.60%, FIG. 1d) and the MFI (mean fluorescence intensity) (2.74-fold increase; FIG. 1e) of EGFP⁺ cells compared with AYBEv0.1, indicating the improved transversion activity by introduction of the MPG mutation, N169S.

Furthermore, two parallel rounds of mutagenesis and screening were performed based on AYBEv0.2 to further improve transversion activity. Based on structural analysis (FIG. 4c) and biochemical characterization of MPG, the non-conserved N-terminal region (1-79 aa) has no effect on either base excision or DNA binding activities of the enzyme^{13, 15}, and thus the 78-298 aa region of MPG-N169S was evenly divided into 13 segments (F1-F13, 17 amino acids each) using a recently developed strategy¹⁶.

In Round 1 mutagenesis and screening, 52 mutants with 4 or 5 random amino acid substitutions in each segment (replacing all non-alanine to alanine (X-to-A) and alanine to valine (A-to-V)) distributed near-uniformly in distance were designed and generated, as shown in Table 2.

TABLE 2
AYBE variants with MPG variants in
Round 1 and transversion activity
thereof normalized to MPG-N169S

| AYBE | Amino acid | Transversion |
|---|---|---|
| variants | mutations | activity |
| MPG-N169S | MYFCMSISSQGDGACVL | 1.00 |
| MPG-F1V1 | APAAHLARLALEFFDQP | 0.86 |
| MPG-F1V2 | SPKGHATRAGLEFFAAP | 0.73 |
| MPG-F1V3 | SPKGALTRLGAEAADQP | 0.18 |
| MPG-F1V4 | SAKGHLTALGLAFFDQA | 0.76 |
| MPG-F2V1 | AAPLARAFLAAALARRL | 0.21 |
| MPG-F2V2 | AVPAARAFAGQVAVRRA | 0.19 |
| MPG-F2V3 | VVALVRAALGQVLVRRL | 0.33 |
| MPG-F2V4 | AVPLAAVELGQVLVAAL | 0.18 |
| MPG-F3V1 | PNAAELRARIVEAEAAL | 0.19 |
| MPG-F3V2 | PAGTEARGRIAETEAYA | 0.19 |
| MPG-F3V3 | PNGTALRGRAVATAAYL | 0.17 |
| MPG-F3V4 | ANGTELAGAIVETEVYL | 0.22 |
| MPG-F4V1 | APEDEAAHARAARQAPR | 0.16 |
| MPG-F4V2 | GPAAEAAASRGGRATPR | 0.19 |
| MPG-F4V3 | GAEDAVAHSRGGRQTAR | 0.26 |
| MPG-F4V4 | GPEDEAVHSAGGAQTPA | 0.19 |
| MPG-F5V1 | NRAMFMAPAALAVYIIY | 0.19 |
| MPG-F5V2 | ARGMFMKPGTLYAAIIA | 0.18 |
| MPG-F5V3 | NRGAFAKPGTAYVYAIY | 0.18 |
| MPG-F5V4 | NAGMAMKAGTLYVYIAY | 0.17 |
| MPG-F6V1 | AMYFCMAIAAQADGACV | 0.18 |
| MPG-F6V2 | GMAFCMSISSQGAAACA | 0.18 |
| MPG-F6V3 | GAYFAMSISSAGDGAAV | 0.26 |
| MPG-F6V4 | GMYACASASSQGDGVCV | 0.19 |
| MPG-F7V1 | AARALEPLEALEAMRAL | 0.21 |
| MPG-F7V2 | LLRAAEPAEGAETMRQA | 0.18 |
| MPG-F7V3 | LLRALAPLAGLATARQL | 0.18 |
| MPG-F7V4 | LLAVLEALEGLETMAQL | 0.18 |
| MPG-F8V1 | RATLRAATAARVLADRE | 1.24 |
| MPG-F8V2 | RSALRKGAASRALKARE | 0.92 |
| MPG-F8V3 | RSTARKGTVSRVAKDRA | 0.80 |
| MPG-F&V4 | ASTLAKGTASAVLKDAE | 0.19 |
| MPG-F9V1 | LCAGPAALCQALAINAA | 0.20 |
| MPG-F9V2 | ACSAPSKLCAALAIAKS | 0.20 |
| MPG-F9V3 | LASGPSKAAQAAAINKS | 0.16 |
| MPG-F9V4 | LCSGASKLCQVLVANKS | 0.15 |
| MPG-F10V1 | FAQRALAQAEAAWLERA | 0.21 |
| MPG-F10V2 | FDARDAAADEAVWAERG | 0.26 |
| MPG-F10V3 | ADQRDLVQDAAVWLARG | 0.23 |
| MPG-F10V4 | FDQADLAQDEVVALEAG | 0.48 |
| MPG-F11V1 | PLEPAEPAAAAAARAAV | 0.16 |
| MPG-F11V2 | PAAPSAPAVVAAARVGA | 0.21 |
| MPG-F11V3 | ALEASEAVVVAAARVGV | 0.80 |
| MPG-F11V4 | PLEPSEPAVVVVVAVGV | 0.16 |
| MPG-F12V1 | AHAAEWARAPLRFAVRA | 0.18 |
| MPG-F12V2 | GAAGEWARKPARAYARG | 0.18 |
| MPG-F12V3 | GHVGAWVRKALRFYVRG | 0.25 |
| MPG-F12V4 | GHAGEAAAKPLAFYVAG | 0.17 |
| MPG-F13V1 | APWVAVVARVAEQAAQA | 0.18 |
| MPG-F13V2 | SPWASAADRAAEQDTQA | 0.45 |
| MPG-F13V3 | SAWVSVVDRVAAADTAA | 0.54 |
| MPG-F13V4 | SPAVSVVDAVVEQDTQV | 0.66 |

In Round 2 mutagenesis and screening, the MPG-N169S protein was scanned with sequential arginine substitutions (X-to-R) or R-to-K substitutions, aiming to enhance the MPG interaction with the substrate DNA (FIG. 1f).

TABLE 3
AYBE variants with MPG variants in Round 2 and transversion
activity thereof normalized to MPG-N169S

| | Trans- | | Trans- | | Trans- |
|---|---|---|---|---|---|
| ΛYBE | version | ΛYBE | version | ΛYBE | version |
| variants | activity | variants | activity | variants | activity |
| MPG-N169S | 1.00 | M151R | 0.35 | L225R | 0.17 |
| S78R | 1.04 | K152R | 0.84 | A226R | 1.10 |
| P79R | 1.01 | P153R | 0.34 | I227R | 0.15 |
| K80R | 1.15 | G154R | 0.17 | N228R | 1.12 |
| G81R | 1.01 | T155R | 0.31 | K229R | 1.06 |
| H82R | 0.26 | L156R | 0.15 | S230R | 1.23 |
| L83R | 0.71 | Y157R | 0.15 | F231R | 0.34 |
| T84R | 0.76 | V158R | 0.15 | D232R | 0.19 |
| R85K | 0.65 | Y159R | 0.18 | Q233R | 0.79 |
| L86R | 0.44 | I160R | 0.72 | R234K | 0.76 |
| G87R | 0.55 | I161R | 0.18 | D235R | 0.48 |
| L88R | 0.60 | Y162R | 0.30 | L236R | 0.14 |
| E89R | 0.91 | G163R | 2.10 | A237R | 0.89 |
| F90R | 0.39 | M164R | 0.50 | Q238R | 1.08 |
| F91R | 0.15 | Y165R | 0.22 | D239R | 0.68 |
| D92R | 0.71 | F166R | 0.55 | E240R | 1.10 |
| Q93R | 0.67 | C167R | 0.15 | A241R | 1.04 |
| P94R | 0.82 | M168R | 0.16 | V242R | 0.16 |
| A95R | 0.13 | S169R | 0.14 | W243R | 0.74 |
| V96R | 0.84 | I170R | 0.15 | L244R | 0.15 |
| P97R | 0.79 | S171R | 0.16 | E245R | 0.58 |
| L98R | 0.16 | S172R | 0.14 | R246K | 1.12 |
| A99R | 0.17 | Q173R | 1.06 | G247R | 0.78 |
| R100K | 0.97 | G174R | 1.27 | P248R | 0.98 |
| A101R | 0.77 | D175R | 1.21 | L249R | 1.02 |
| F102R | 0.22 | G176R | 0.14 | E250R | 0.97 |
| L103R | 0.15 | A177R | 1.02 | P251R | 1.02 |
| G104R | 0.73 | C178R | 0.13 | S252R | 0.99 |
| Q105R | 0.89 | V179R | 0.13 | E253R | 1.10 |
| V106R | 0.78 | L180R | 0.15 | P254R | 1.07 |
| L107R | 0.20 | L181R | 0.16 | A255R | 1.05 |
| V108R | 0.55 | R182K | 0.14 | V256R | 0.76 |
| R109K | 0.79 | A183R | 0.17 | V257R | 0.69 |
| R110K | 1.04 | L184R | 0.15 | A258R | 0.89 |
| L111R | 0.83 | E185R | 1.08 | A259R | 0.63 |
| P112R | 0.92 | P186R | 0.17 | A260R | 0.81 |
| N113R | 0.74 | L187R | 1.07 | R261K | 0.14 |
| G114R | 0.88 | E188R | 1.13 | V262R | 0.35 |
| T115R | 1.08 | G189R | 0.99 | G263R | 0.27 |
| E116R | 1.01 | L190R | 1.12 | V264R | 0.15 |
| L117R | 0.20 | E191R | 1.14 | G265R | 0.66 |
| R118K | 0.98 | T192R | 1.12 | H266R | 0.74 |
| G119R | 0.20 | M193R | 0.14 | A267R | 0.53 |
| R120K | 1.05 | R194K | 0.98 | G268R | 0.93 |
| I121R | 0.20 | Q195R | 1.04 | E269R | 0.98 |
| V122R | 0.68 | L196R | 0.99 | W270R | 0.14 |
| E123R | 0.15 | R197K | 0.82 | A271R | 0.58 |
| T124R | 0.15 | S198R | 1.11 | R272K | 1.12 |
| E125R | 0.15 | T199R | 1.18 | K273R | 0.92 |
| A126R | 0.15 | L200R | 0.83 | P274R | 1.05 |
| Y127R | 0.14 | R201K | 1.04 | L275R | 0.13 |
| L128R | 0.61 | K202R | 1.12 | R276K | 0.23 |
| G129R | 0.14 | G203R | 0.98 | F277R | 0.57 |
| P130R | 0.70 | T204R | 1.00 | Y278R | 0.21 |
| E131R | 0.76 | A205R | 0.95 | V279R | 1.12 |
| D132R | 0.15 | S206R | 0.99 | R280K | 1.03 |
| E133R | 0.90 | R207K | 0.79 | G281R | 1.05 |
| A134R | 0.21 | V208R | 1.10 | S282R | 0.66 |
| A135R | 0.14 | L209R | 0.94 | P283R | 0.89 |
| H136R | 0.15 | K210R | 1.11 | W284R | 0.24 |
| S137R | 0.15 | D211R | 0.97 | V285R | 0.54 |
| R138K | 1.06 | R212K | 1.03 | S286R | 0.32 |
| G139R | 0.58 | E213R | 0.98 | V287R | 0.66 |
| G140R | 0.48 | L214R | 0.15 | V288R | 0.90 |
| R141K | 0.98 | C215R | 0.15 | D289R | 0.59 |
| Q142R | 0.84 | S216R | 1.07 | R290K | 0.96 |
| T143R | 0.39 | G217R | 0.16 | V291R | 1.12 |
| P144R | 0.99 | P218R | 0.15 | A292R | 0.92 |
| R145K | 1.00 | S219R | 0.16 | E293R | 0.82 |
| N146R | 0.00 | K220R | 1.01 | Q294R | 1.19 |
| R147K | 0.86 | L221R | 0.17 | D295R | 1.21 |
| G148R | 0.14 | C222R | 0.15 | T296R | 1.14 |
| M149R | 0.14 | Q223R | 0.79 | Q297R | 1.09 |
| F150R | 0.15 | A224R | 0.16 | A298R | 1.12 |

Among others, the AYBE variant v1 (AYBEv1, SEQ ID NO: 31) containing MPG-F8V1 (MPGv1, MPG-N169S+S198A+K202A+G203A+S206A+K210A, SEQ ID NO: 30) from Round 1 and the AYBE variant v2 (AYBEv2, SEQ ID NO: 33) containing MPG-G163R+N169S (MPGv2, SEQ ID NO: 32) from Round 2 showed the highest transversion activity in each Round (FIG. 1g-h). AYBEv1 and AYBEv2 exhibited 1.24- and 2.10-fold increase of transversion activity compared with AYBEv0.2, respectively (FIG. 1g-h), and 2.83- and 3.83-fold increase of transversion activity compared with AYBEv0.1, respectively (FIG. 1i).

To investigate the additive effect of the MPG mutations in MPGv1 and MPGv2, the mutations in MPGv1 and MPGv2 were combined in Round 3 into MPGv3 (MPG-G163R+N169S+S198A+K202A+G203A+S206A+K210A, SEQ ID NO: 34) to construct AYBEv3 (SEQ ID NO: 35) containing MPGv3, and surprisingly, synergistic improvement of transversion activity of 4.78-fold compared with AYBEv0.1 was achieved in view of the improvement of 2.83-fold by AYBEv1 alone and 3.83-fold by AYBEv2 alone compared with AYBEv0.1 (FIG. 1i).

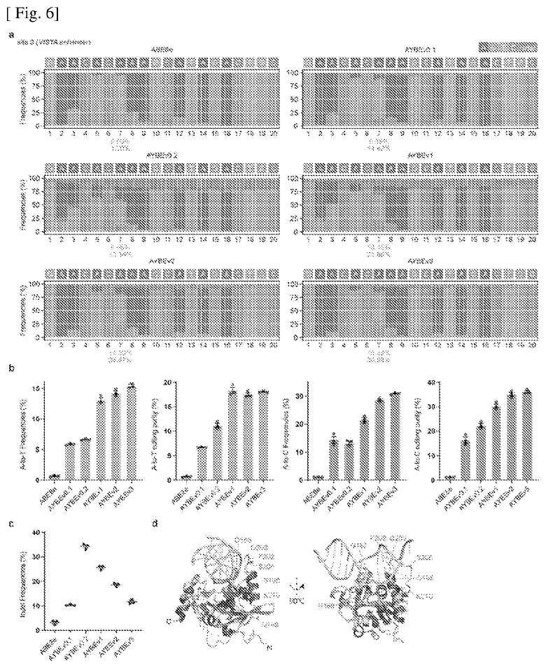

The improvement of the transversion activity by the progressive AYBE variants was validated at an endogenous genomic site using fluorescence-activated cell sorting (FACS) (FIGS. 6a and 6b).

Similar to the synergistic improvement of the transversion activity (from 5.88% to 15.49% for A-to-T transversion and from 14.42% to 30.98% for A-to-C transversion), synergistic reduction of indel (insertion and/or deletion) frequencies (indicating undesired DNA cleavage) was also observed for AYBEv3 (from 34.28% to 11.64%) (FIG. 6c), indicating improved safety. Without wishing to be bound to theory, it is believed that the MPG mutations in AYBEv3 might facilitate specific substrate selection or modulate the DNA-binding activity of MPG protein (FIG. 6d).

In conclusion, the results above indicate effective optimization of AYBE toward high activity for A-to-T and A-to-C transversion editing.

### Example 3 Evaluation of On-Target Editing Profiles of AYBEv3

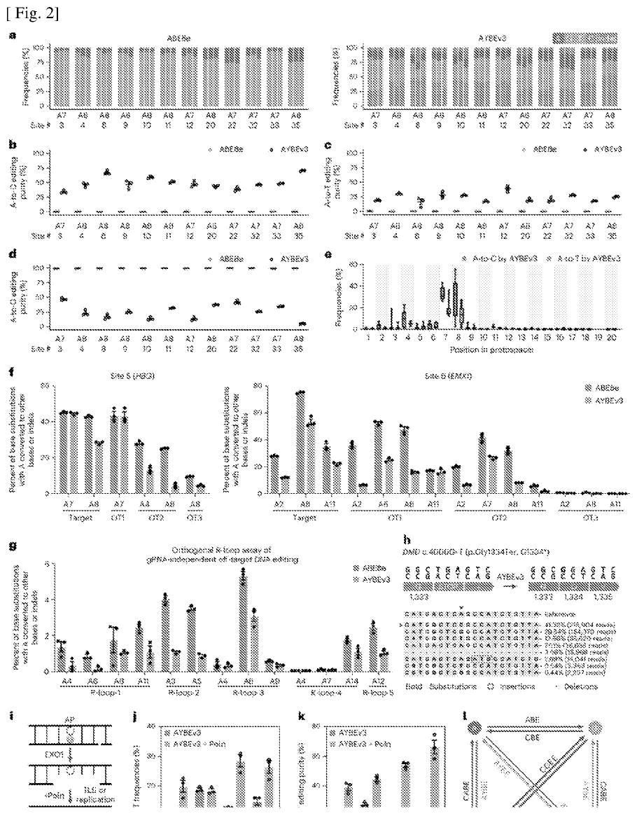

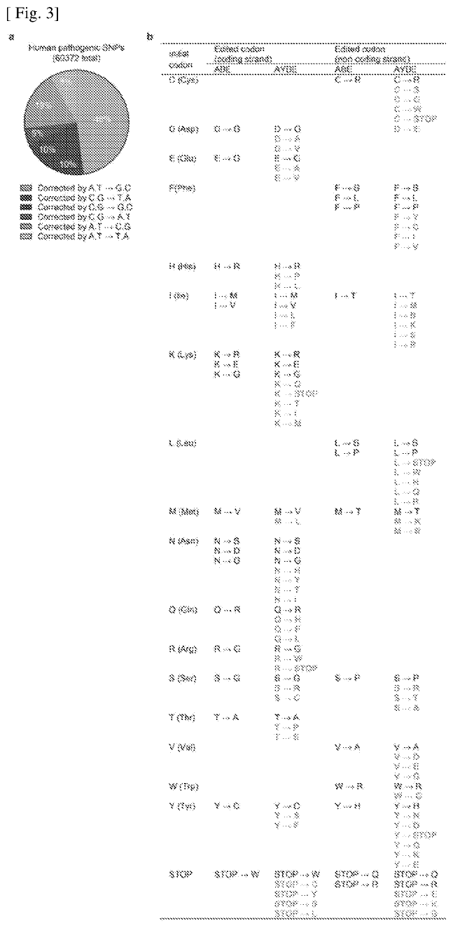

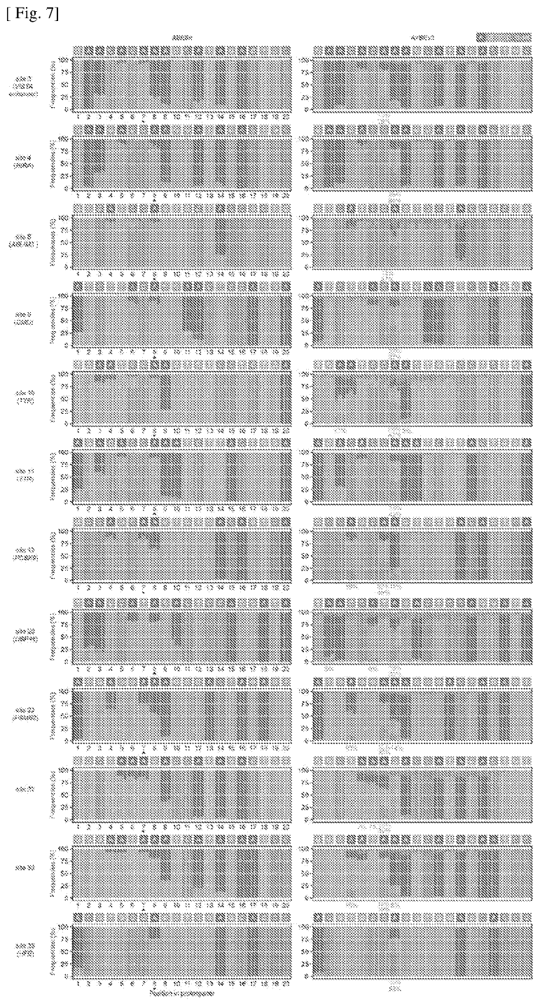

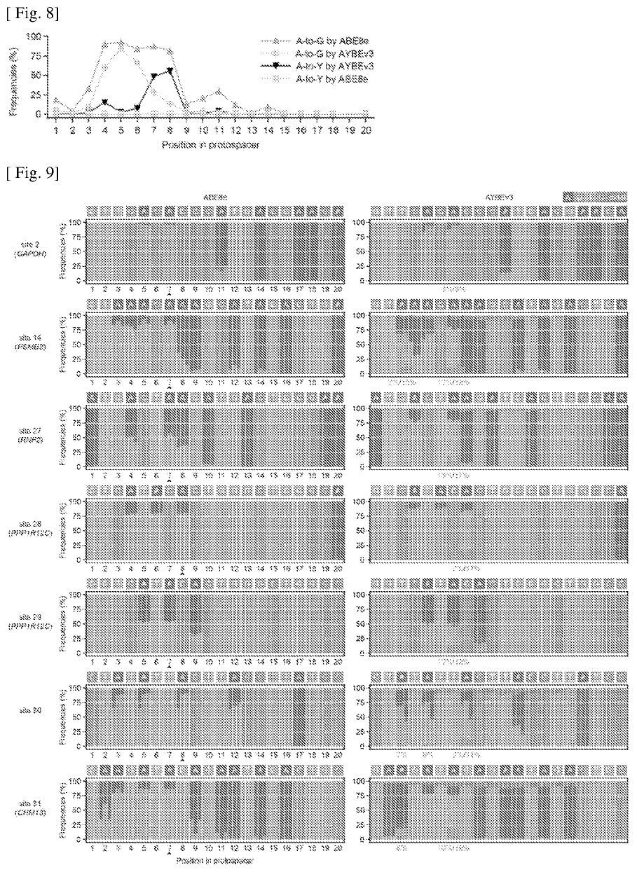

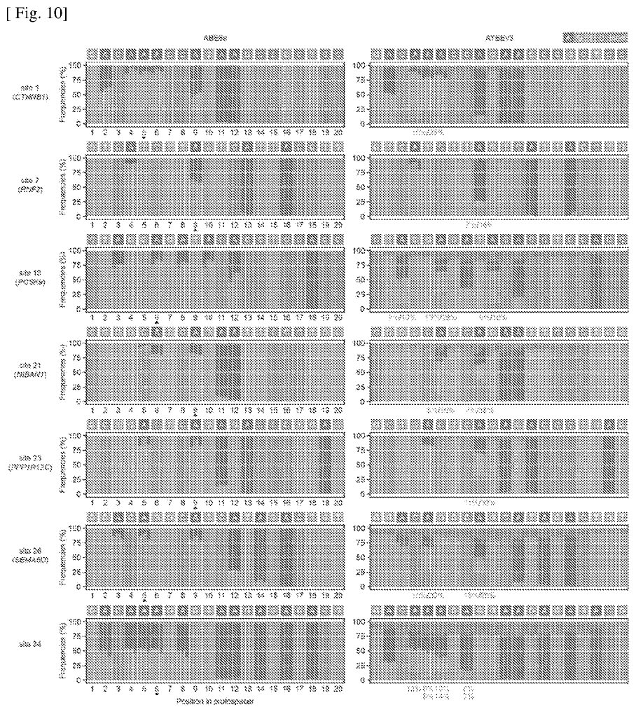

The editing profiles of AYBEv3 was further characterized by targeting dozens of endogenous genomic loci. Efficient A-to-C or A-to-T edits were observed with AYBEv3, but almost no A-to-Y (A-to-C or A-to-T) transversion editing at any position of the 26 sites tested with ABE8e (FIG. 7-10). The top 12 efficiently edited sites included five sites with an A7 and seven sites with an A8 (FIG. 2a and FIG. 7), with A-to-C edits as the predominant product (mean editing frequencies ranging from 34.14 to 70%, up to 70% purity for site 35), with the mean editing frequencies of A-to-T edits ranging from 16.29 to 39.09% (up to 39.09% purity for site 12) (FIG. 2b-d), indicating that AYBEv3 exhibited high editing efficiency for A-to-Y transversion at protospacer positions 7 and 8 (mean editing frequencies ranging from 8 to 72%), including 3 to 53% editing efficiency for A-to-C transversion and 3 to 32% for A-to-T transversion (FIG. 2a and FIG. 7).

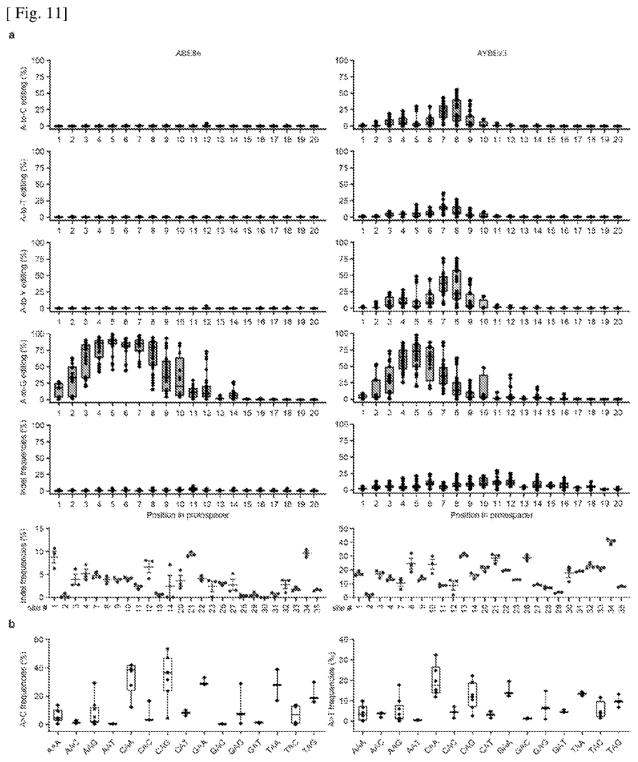

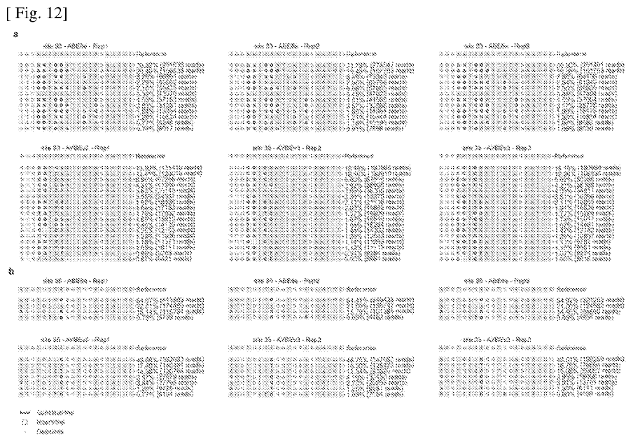

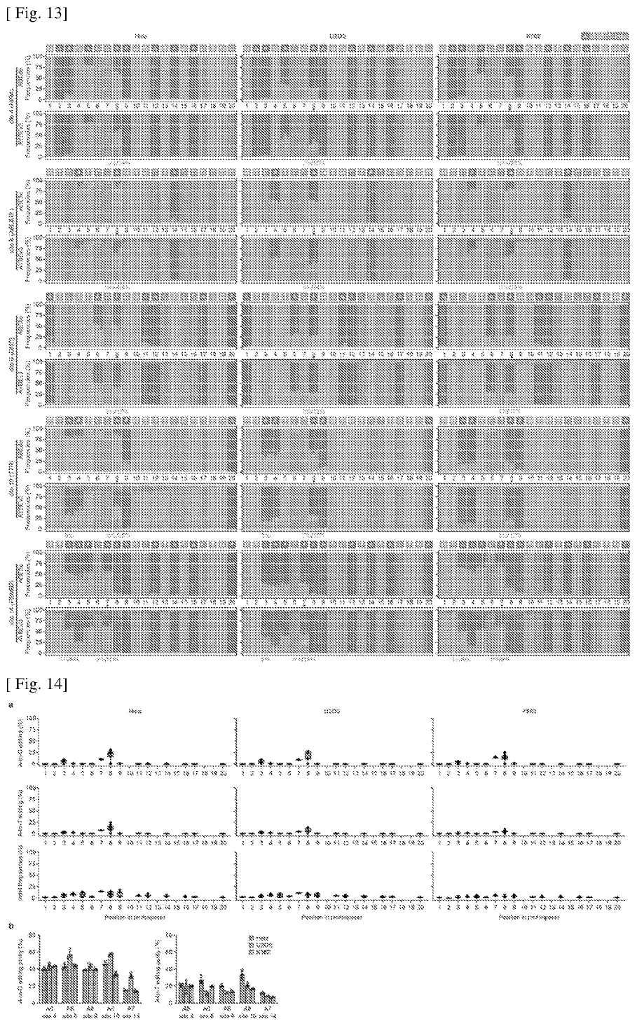

Overall, it showed that editing window of AYBEv3 existed at positions 3 to 10 or preferably positions 5 to 9 on the protospacer and that indels were distributed throughout the protospacer (FIG. 11a), with CAA and CAG as the top two preferred editing motifs (FIG. 11b). Note that AYBEv3 induced mean indel frequencies (percentage of alleles that contain an insertion or deletion across the entire protospacer) ranging from 1.63% to 40.68% (FIG. 11a). In addition, analysis of allele compositions showed that AYBEv3 induced less bystander editing than ABE8e (FIG. 12). Moreover, AYBEv3 also exhibited efficient A-to-C and A-to-T transversion editing activity at protospacer positions 7 and 8, with A-to-C edits as the predominant product, across three different human cell lines (HeLa, U2OS, and K562 cells) (FIG. 13 and FIG. 14).

### Example 4 Evaluation of Off-Target Editing of AYBEv3

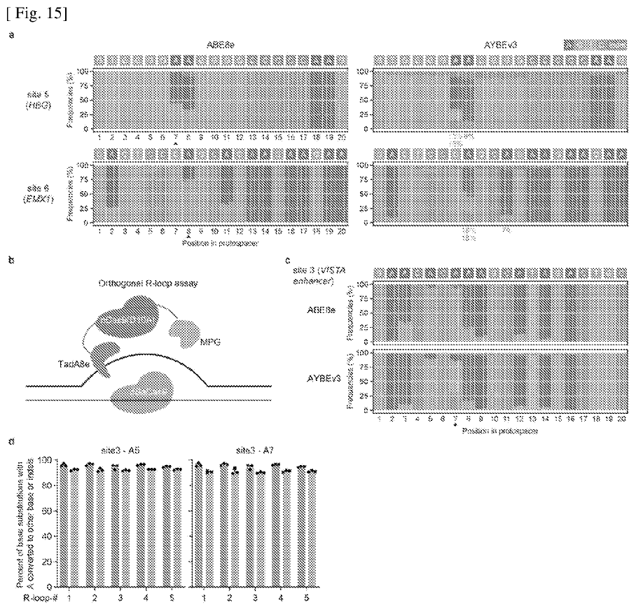

To investigate the off-target effect of AYBE, gRNA-dependent off-target (OT) activity of AYBEv3 was analyzed at two previously reported gRNA-dependent off-target (OT) sites (FIG. 2f), and the ability of AYBEv3 to mediate gRNA-independent off-target DNA editing was characterized by using orthogonal R-loop assay in five dSaCas9 R-loops¹⁷ (FIG. 2g). A decrease in editing at all six gRNA-dependent off-target sites and all five guide-independent off-target sites was observed when comparing AYBEv3 to ABE8e (FIGS. 2f and 2g and FIG. 15).

### Example 5 Evaluation of Therapeutic Potential of AYBEv3

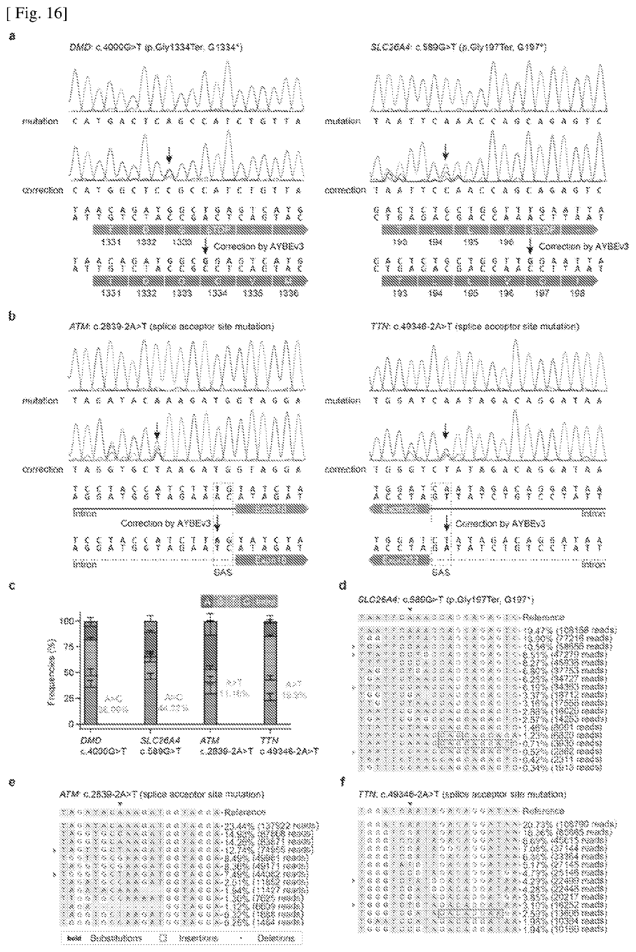

In addition, a proof-of-concept (POC) study was performed to investigate the therapeutic potential of AYBEv3 for correcting disease-related transversion mutations. By testing two nonsense mutations and two splicing acceptor site mutations with AYBEv3 in a stable HEK293T cell lines generated via lentiviral transduction, about 36% and 44% correction frequencies of A-to-C edits at DMD and SLC26A4 nonsense mutation sites (spacer sequences of SEQ ID NOs: 75 and 77, respectively) and about 11% and 20% correction frequencies of A-to-T edits at ATM and TTN splicing acceptor site mutations (spacer sequences of SEQ ID NOs: 76 and 78, respectively) were observed, respectively (FIG. 2h and FIG. 16), indicating promising potential for AYBE in both basic research and therapeutic applications.

### Example 6 Improved Transversion Purity by Introduction of a TLS Polymerase for More Precise Editing

In the AYBE-mediated transversion editing process, cellular DNA repair machinery was channeled to favor base excision repair (BER) pathway by the activity of hypoxanthine excision repair proteins after adenine deamination.

Human Polη (SEQ ID NO: 36), a translesion synthesis (TLS) polymerase preferentially incorporating dA opposite AP sites18, was co-expressed with AYBEv3 to increase the percentage or purity of A-to-T editing (FIG. 2i-k, FIG. 17). After co-transfection of plasmids encoding AYBEv3 and Polη separately, the purity of A-to-T editing outcomes was significantly increased and achieved up to 66% (FIG. 2k). Polη was expressed with a plasmid comprising a polynucleotide encoding Polη under the regulation of CAG promoter and followed by a sequence encoding bGH polyA signal.

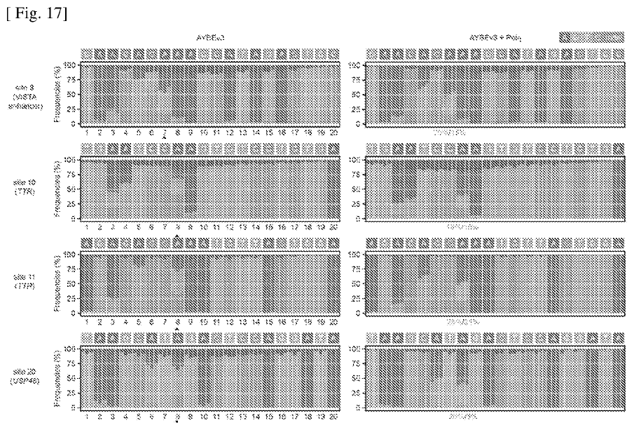

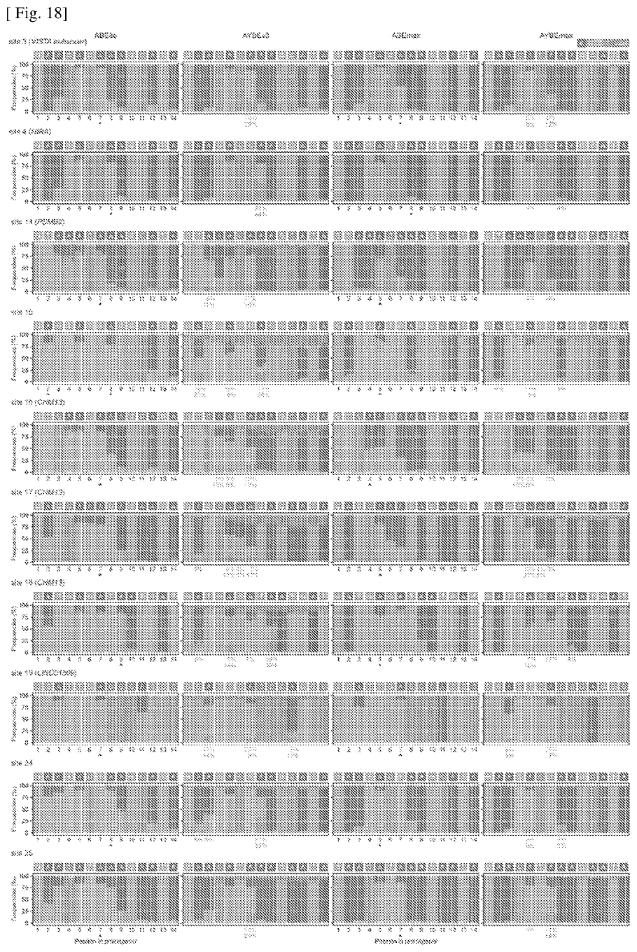

AYBEv3 was also tested with a less processive deaminase TadA7.10 from ABEmax, termed AYBEmax, and it was found that AYBEmax did not lead to more dominant A>T or A>C outcome (FIG. 18).

### Example 7 Alternative Nucleic Acid Programmable DNA Binding Domain (napDNAbd)

To expand the scope for selecting the napDNAbd of the AYBE of the disclosure, the Cas9 nickase (SpCas9-D10A, SEQ ID NO: 4) in AYBEv0.2 and AYBEv3 was replaced with a dead Cas9 (SpCas9-D10A+H840A, SEQ ID NO: 37) or a Cas12i nickase (nCas12imax (SiCas12i-N243R+E336R), SEQ ID NO: 38; corresponding scaffold sequence of SEQ ID NO: 39) to evaluate the transversion activity in the A-to-T reporter system.

It was observed (FIG. 19) that a quite high transversion activity of about 90% was achieved with the use of dead Cas9 in place of Cas9 nickase, indicating that the AYBE of the disclosure can not only use a Cas nickase but also a dead Cas (inactivate) substantially lacking endonuclease activity.

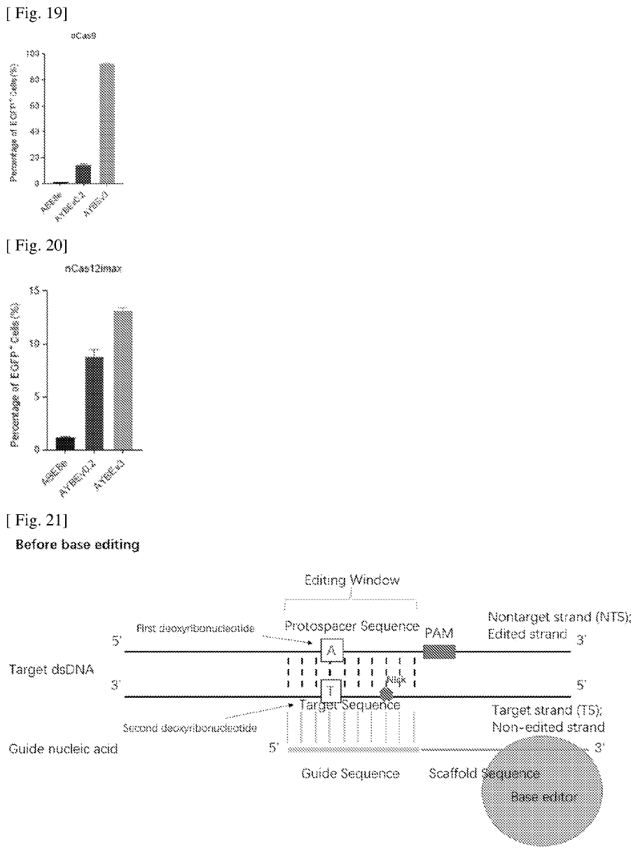

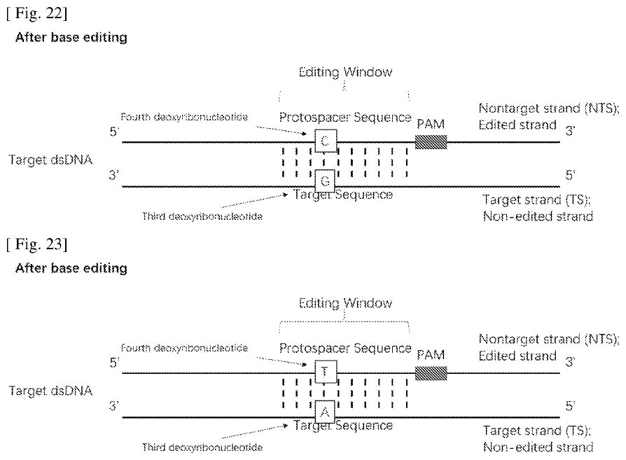

It was also observed (FIG. 20) that a significant transversion activity of about 14% was achieved with the use of Cas12i nickase in place of Cas9 nickase, indicating that the AYBE of the disclosure is not limited to a particular type of Cas (i.e., Cas9) but can also make use of additional effector proteins that could serve as a nucleic acid programmable DNA binding domain, such as, Cas12, IscB.

### REFERENCES

1. Porto, E. M., Komor, A. C., Slaymaker, I. M. &Yeo, G. W. Base editing: advances and therapeutic opportunities. Nat Rev Drug Discov 19, 839-859 (2020).
2. Rees, H. A. &Liu, D. R. Base editing: precision chemistry on the genome and transcriptome of living cells. Nat Rev Genet 19, 770-788 (2018).
3. Gaudelli, N. M. et al. Programmable base editing of A*T to G*C in genomic DNA without DNA cleavage. Nature 551, 464-471 (2017).
4. Komor, A. C., Kim, Y. B., Packer, M. S., Zuris, J. A. &Liu, D. R. Programmable editing of a target base in genomic DNA without double-stranded DNA cleavage. Nature 533, 420-424 (2016).
5. Zhao, D. et al. Glycosylase base editors enable C-to-A and C-to-G base changes. Nat Biotechnol 39, 35-40 (2021).
6. Kurt, I. C. et al. CRISPR C-to-G base editors for inducing targeted DNA transversions in human cells. Nat Biotechnol 39, 41-46 (2021).
7. Koblan, L. W. et al. Efficient C*G-to-G*C base editors developed using CRISPRi screens, target-library analysis, and machine learning. Nat Biotechnol 39, 1414-1425 (2021).
8. Chen, L. et al. Programmable C: G to G: C genome editing with CRISPR-Cas9-directed base excision repair proteins. Nat Commun 12, 1384 (2021).
9. Yuan, T. et al. Optimization of C-to-G base editors with sequence context preference predictable by machine learning methods. Nat Commun 12, 4902 (2021).
10. Robertson, A. B., Klungland, A., Rognes, T. &Leiros, I. DNA repair in mammalian cells: Base excision repair: the long and short of it. Cell Mol Life Sci 66, 981-993 (2009).
11. Hindi, N. N., Elsakrmy, N. &Ramotar, D. The base excision repair process: comparison between higher and lower eukaryotes. Cell Mol Life Sci 78, 7943-7965 (2021).
12. Saparbaev, M. &Laval, J. Excision of hypoxanthine from DNA containing dIMP residues by the Escherichia coli, yeast, rat, and human alkylpurine DNA glycosylases. Proc Natl Acad Sci USA 91, 5873-5877 (1994).
13. Lau, A. Y., Scharer, O. D., Samson, L., Verdine, G. L. &Ellenberger, T. Crystal structure of a human alkylbase-DNA repair enzyme complexed to DNA: mechanisms for nucleotide flipping and base excision. Cell 95, 249-258 (1998).
14. Connor, E. E. &Wyatt, M. D. Active-site clashes prevent the human 3-methyladenine DNA glycosylase from improperly removing bases. Chem Biol 9, 1033-1041 (2002).
15. Vallur, A. C., Maher, R. L. &Bloom, L. B. The efficiency of hypoxanthine excision by alkyladenine DNA glycosylase is altered by changes in nearest neighbor bases. DNA Repair (Amst) 4, 1088-1098 (2005).
16. Tong, H. et al. High-fidelity Cas13 variants for targeted RNA degradation with minimal collateral effects. Nat Biotechnol (2022).
17. Richter, M. F. et al. Phage-assisted evolution of an adenine base editor with improved Cas domain compatibility and activity. Nat Biotechnol 38, 883-891 (2020).
18. Choi, J. Y., Lim, S., Kim, E. J., Jo, A. &Guengerich, F. P. Translesion synthesis across abasic lesions by human B-family and Y-family DNA polymerases alpha, delta, eta, iota, kappa, and REV1. J Mol Biol 404, 34-44 (2010).
19. Thompson, P. S. &Cortez, D. New insights into abasic site repair and tolerance. DNA Repair (Amst) 90, 102866 (2020).
20. Clement, K. et al. CRISPResso2 provides accurate and rapid genome editing sequence analysis. Nat Biotechnol 37, 224-226 (2019).

Various modifications and variations of the described products, methods, and uses of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

SEQUENCES
wild type human MPG with N-terminal starting Methionine (M), full
length, 298 aa
SEQ ID NO: 1
MVTPALQMKKPKQFCRRMGQKKQRPARAGQPHSSSDAAQAPAEQPHSSSDAAQAPCPRERCLGPPTTPG
PYRSIYFSSPKGHLTRLGLEFFDQPAVPLARAFLGQVLVRRLPNGTELRGRIVETEAYLGPEDEAAHSRGG
RQTPRNRGMFMKPGTLYVYIIYGMYFCMNISSQGDGACVLLRALEPLEGLETMRQLRSTLRKGTASRVL
KDRELCSGPSKLCQALAINKSFDQRDLAQDEAVWLERGPLEPSEPAVVAAARVGVGHAGEWARKPLRFY
VRGSPWVSVVDRVAEQDTQA,
wild type human MPG without N-terminal starting Methionine (M)
(“MPG” for short), 297 aa
SEQ ID NO: 2
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYGMYFCMNISSQGDGACVLLRALEPLEGLETMRQLRSTLRKGTASRVLK
DRELCSGPSKLCQALAINKSFDQRDLAQDEAVWLERGPLEPSEPAVVAAARVGVGHAGEWARKPLRFYV
RGSPWVSVVDRVAEQDTQA,
TadA8e without N-terminal starting Methionine (M)
SEQ ID NO: 3
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNSKRGAAGSLMNVLNYPGMNHRVEITEGILA
DECAALLCDFYRMPRQVFNAQKKAQSSIN,
SpCas9-D10A, Cas9 nickase, nCas9, without N-terminal starting
Methionine (M)
SEQ ID NO: 4
DKKYSIGLAIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNEDLAEDAKLQLSKDTYDDDLDNLLAQIGD
QYADLFLAAKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTEDNGSIPHQIHLGELHAILRRQE
DFYPFLKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVKQLKED
YFKKIECFDSVEISGVEDRENASLGTYHDLLKIIKDKDFLDNEENEDILEDIVLTLTLFEDREMIEERLKTYA
HLFDDKVMKQLKRRRYTGWGRLSRKLINGIRDKQSGKTILDELKSDGFANRNFMQLIHDDSLTFKEDIQK
AQVSGQGDSLHEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDTTIDRKRYTSTKEV
LDATLIHQSITGLYETRIDLSQLGGD,
prototype AYBE, MTC, full length with N-terminal starting
Methionine (M)
SEQ ID NO: 5
MKRTADGSEFESPKKKRKVVTPALQMKKPKQFCRRMGQKKQRPARAGQPHSSSDAAQAPAEQPHS
SSDAAQAPCPRERCLGPPTTPGPYRSIYFSSPKGHLTRLGLEFFDQPAVPLARAFLGQVLVRRLPNGT
ELRGRIVETEAYLGPEDEAAHSRGGRQTPRNRGMFMKPGTLYVYIIYGMYFCMNISSQGDGACVL
LRALEPLEGLETMRQLRSTLRKGTASRVLKDRELCSGPSKLCQALAINKSFDQRDLAQDEAVWLER
GPLEPSEPAVVAAARVGVGHAGEWARKPLRFYVRGSPWYSYVDRVAEQDTQALGGDSGGSGGSGGS
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNSKRGAAGSLMNVLNYPGMNHRVEITEGILA
DECAALLCDFYRMPRQVFNAQKKAQSSINSGGSSGGSSGSETPGTSESATPESSGGSSGGSDKKYSIGLAI
GTNSVGWAVITDEYKVPSKKFKVLGNTDRHSIKKNLIGALLEDSGETAEATRLKRTARRRYTRRKNRICYL
QEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQSKNGYAGYIDGGA
SQEEFYKFIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYAHLEDDKVMKQL
KRRRYTGWGRLSRKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKRPLIETNGETGEI
VWDKGRDFATVRKVLSMPQVNIVKKTEVQTGGESKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKEVLDATLIHQSITGL
YETRIDLSQLGGDSGGSKRTADGSEFEPKKKRKV,
prototype AYBE, TMC, full length with N-terminal starting
Methionine (M)
SEQ ID NO: 6
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINLGGDSGGSGGSGGSVTPALQ
MKKPKQFCRRMGQKKQRPARAGQPHSSSDAAQAPAEQPHSSSDAAQAPCPRERCLGPPTTPGPYR
SIYFSSPKGHLTRLGLEFFDQPAVPLARAFLGQVLVRRLPNGTELRGRIVETEAYLGPEDEAAHSRG
GRQTPRNRGMFMKPGTLYVYIIYGMYFCMNISSQGDGACVLLRALEPLEGLETMRQLRSTLRKGT
ASRVLKDRELCSGPSKLCQALAINKSFDQRDLAQDEAVWLERGPLEPSEPAVVAAARVGVGHAGEW
ARKPLRFYVRGSPWVSVVDRVAEQDTQASGGSSGGSSGSETPGTSESATPESSGGSSGGSDKKYSIGLAI
GTNSVGWAVITDEYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENL
LAQLPGEKKNGLFGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQSKNGYAGYIDGGA
SQEEFYKFIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLILTLFEDREMIEERLKTYAHLFDDKVMKQL
KRRRYTGWGRLSRKLINGIRDKQSGKTILDELKSDGFANRNFMQLIHDDSLTFKEDIQKAQVSGQGDSLH
EHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKKYGGEDSPTVAY
SVLVVAKVEKGKSKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDEIIEQISEFSKRVILA
DANLDKVLSAYNKHRDKPIREQAENIIHLETLTNLGAPAAFKYFDTTIDRKRYTSTKEVLDATLIHQSITGL
YETRIDLSQLGGDSGGSKRTADGSEFEPKKKRKV,
prototype AYBE, TCM (AYBEv0.1),, full length with N-terminal
starting Methionine (M)
SEQ ID NO: 7
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINSGGSSGGSSGSETPGTSESATPE
SSGGSSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRENASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNEMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLEVEQ
HKHYLDEIIEQISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSKRTADGSEFEPKKKRKVLGGDSGGSGGSGGSVT
PALQMKKPKQFCRRMGQKKQRPARAGQPHSSSDAAQAPAEQPHSSSDAAQAPCPRERCLGPPTTP
GPYRSIYFSSPKGHLTRLGLEFFDQPAVPLARAFLGQVLVRRLPNGTELRGRIVETEAYLGPEDEAA
HSRGGRQTPRNRGMFMKPGTLYVYHYGMYFCMNISSQGDGACVLLRALEPLEGLETMRQLRSTL
RKGTASRVLKDRELCSGPSKLCQALAINKSFDQRDLAQDEAVWLERGPLEPSEPAVVAAARVGVGH
AGEWARKPLRFYVRGSPWVSVVDRVAEQDTQASGGSKRTADGSEFEPKKKRKV,
dMPG (dead MPG, inactivated MPG-E125A, Y127A, H136A mutant)
SEQ ID NO: 8
VTPALQMKKPKQFCRRMGQKKQRPARAGQPHSSSDAAQAPAEQPHSSSDAAQAPCPRERCLGPPTTPGP
YRSIYESSPKGHLTRLGLEFFDQPAVPLARAFLGQVLVRRLPNGTELRGRIVETAAALGPEDEAAASRGGR
QTPRNRGMFMKPGTLYVYIIYGMYFCMNISSQGDGACVLLRALEPLEGLETMRQLRSTLRKGTASRVLK
DRELCSGPSKLCQALAINKSFDQRDLAQDEAVWLERGPLEPSEPAVVAAARVGVGHAGEWARKPLRFYV
RGSPWVSVVDRVAEQDTQA,
AYBE-dMPG (negative control)
SEQ ID NO: 9
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINSGGSSGGSSGSETPGTSESATPE
SSGGSSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDELDNEENEDILEDIVLTLTLFEDREMI
EERLKTYAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSKRTADGSEFEPKKKRKVLGGDSGGSGGSGGSVT
PALQMKKPKQFCRRMGQKKQRPARAGQPHSSSDAAQAPAEQPHSSSDAAQAPCPRERCLGPPTTP
GPYRSIYFSSPKGHLTRLGLEFFDQPAVPLARAFLGQVLVRRLPNGTELRGRIVET [symbol] A [symbol] LGPEDEAA [symbol]
SRGGRQTPRNRGMFMKPGTLYVYIIYGMYFCMNISSQGDGACVLLRALEPLEGLETMRQLRSTL
RKGTASRVLKDRELCSGPSKLCQALAINKSFDQRDLAQDEAVWLERGPLEPSEPAVVAAARVGVGH
AGEWARKPLRFYVRGSPWVSVVDRVAEQDTQASGGSKRTADGSEFEPKKKRKV,
ABE8e (blank control)
SEQ ID NO: 10
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINSGGSSGGSSGSETPGTSESATPE
SSGGSSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYEKKIECFDSVEISGVEDRENASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKEDNLTKAERGGLS
ELDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSKRTADGSEFEPKKKRKV,
bpNLS 1
SEQ ID NO: 11
KRTADGSEFESPKKKRKV,
bpNLS 2
SEQ ID NO: 12
KRTADGSEFEPKKKRKV,
MPG-N169S (without N-terminal starting Methionine (M); N169S
is numbered based on the full-length wild type human MPG of SEQ ID NO: 1)
SEQ ID NO: 28
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYGMYFCMSISSQGDGACVLLRALEPLEGLETMRQLRSTLRKGTASRVLK
DRELCSGPSKLCQALAINKSFDQRDLAQDEAVWLERGPLEPSEPAVVAAARVGVGHAGEWARKPLRFYV
RGSPWVSVVDRVAEQDTQA,
AYBEv0.2
SEQ ID NO: 29
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMN
VLNYPGMNHRVETTEGILADECAALLCDFYRMPRQVFNAQKKAQSSINSGGSSGGSSGSETPGTSESATPE
SSGGSSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKERGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSKRTADGSEFEPKKKRKVLGGDSGGSGGSGGSVT
PALQMKKPKQFCRRMGQKKQRPARAGQPHSSSDAAQAPAEQPHSSSDAAQAPCPRERCLGPPTTP
GPYRSIYESSPKGHLTRLGLEFFDQPAVPLARAFLGQVLVRRLPNGTELRGRIVETEAYLGPEDEAA
HSRGGRQTPRNRGMFMKPGTLYVYIIYGMYFCMSISSQGDGACVLLRALEPLEGLETMRQLRSTLR
KGTASRVLKDRELCSGPSKLCQALAINKSFDQRDLAQDEAVWLERGPLEPSEPAVVAAARVGVGHA
GEWARKPLRFYVRGSPWVSVVDRVAEQDTQASGGSKRTADGSEFEPKKKRKV,
MPG-N169S + S198A + K202A + G203A + S206A + K210A (MPGv1),
6 mutations, without N-terminal starting Methionine (M)
SEQ ID NO: 30
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYITYGMYFCMSISSQGDGACVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA,
AYBEv1, full length with N-terminal starting Methionine (M)
SEQ ID NO: 31
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINSGGSSGGSSGSETPGTSESATPE
SSGGSSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNEMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSKRTADGSEFEPKKKRKVLGGDSGGSGGSGGSVT
PALQMKKPKQFCRRMGQKKQRPARAGQPHSSSDAAQAPAEQPHSSSDAAQAPCPRERCLGPPTTP
GPYRSIYFSSPKGHLTRLGLEFFDQPAVPLARAFLGQVLVRRLPNGTELRGRIVETEAYLGPEDEAA
HSRGGRQTPRNRGMFMKPGTLYVYIIYGMYFCMSISSQGDGACVLLRALEPLEGLETMRQLRATLRA
ATAARVLADRELCSGPSKLCQALAINKSFDQRDLAQDEAVWLERGPLEPSEPAVVAAARVGVGHAG
EWARKPLRFYVRGSPWVSVVDRVAEQDTQASGGSKRTADGSEFEPKKKRKV,
MPGv2 (MPG-G163R + N169S), 2 mutations, without N-terminal
starting Methionine (M)
SEQ ID NO: 32
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYITYRMYFCMSISSQGDGACVLLRALEPLEGLETMRQLRSTLRKGTASRVLKD
RELCSGPSKLCQALAINKSFDQRDLAQDEAVWLERGPLEPSEPAVVAAARVGVGHAGEWARKPLRFYVR
GSPWVSVVDRVAEQDTQA,
AYBEv2, full length with N-terminal starting Methionine (M)
SEQ ID NO: 33
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINSGGSSGGSSGSETPGTSESATPE
SSGGSSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESELVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKEDNLTKAERGGLS
ELDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSKRTADGSEFEPKKKRKVLGGDSGGSGGSGGSVT
PALQMKKPKQFCRRMGQKKQRPARAGQPHSSSDAAQAPAEQPHSSSDAAQAPCPRERCLGPPTTP
GPYRSIYFSSPKGHLTRLGLEFFDQPAVPLARAFLGQVLVRRLPNGTELRGRIVETEAYLGPEDEAA
HSRGGRQTPRNRGMFMKPGTLYVYIIYRMYFCMSISSQGDGACVLLRALEPLEGLETMRQLRSTLR
KGTASRVLKDRELCSGPSKLCQALAINKSFDQRDLAQDEAVWLERGPLEPSEPAVVAAARVGVGHA
GEWARKPLRFYVRGSPWVSVVDRVAEQDTQASGGSKRTADGSEFEPKKKRKV,
MPGv3 (MPG-G163R + N169S + S198A + K202A + G203A + S206A + K210A),
7 mutations, without N-terminal starting Methionine (M)
SEQ ID NO: 34
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMSISSQGDGACVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA,
AYBEv3, full length with N-terminal starting Methionine (M)
SEQ ID NO: 35
MKRTADGSEFESPKKKRKVSEVEFSHEYWMRHALTLAKRARDEREVPVGAVLVLNNRVIGEGWNRAIGL.
HDPTAHAEIMALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSINSGGSSGGSSGSETPGTSESATPE
SSGGSSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLEGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKEDNLTKAERGGLS
ELDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSKRTADGSEFEPKKKRKVLGGDSGGSGGSGGSVT
PALQMKKPKQFCRRMGQKKQRPARAGQPHSSSDAAQAPAEQPHSSSDAAQAPCPRERCLGPPTTP
GPYRSIYFSSPKGHLTRLGLEFFDQPAVPLARAFLGQVLVRRLPNGTELRGRIVETEAYLGPEDEAA
HSRGGRQTPRNRGMFMKPGTLYVYIIYRMYFCMSISSQGDGACVLLRALEPLEGLETMRQLRATLRA
ATAARVLADRELCSGPSKLCQALAINKSFDQRDLAQDEAVWLERGPLEPSEPAVVAAARVGVGHAG
EWARKPLRFYVRGSPWVSVVDRVAEQDTQASGGSKRTADGSEFEPKKKRKV,
human Polη
SEQ ID NO: 36
MATGQDRVVALVDMDCFFVQVEQRQNPHLRNKPCAVVQYKSWKGGGIIAVSYEARAFGVTRSMWADD
AKKLCPDLLLAQVRESRGKANLTKYREASVEVMEIMSRFAVIERASIDEAYVDLTSAVQERLQKLQGQPIS
ADLLPSTYIEGLPQGPTTAEETVQKEGMRKQGLFQWLDSLQIDNLTSPDLQLTVGAVIVEEMRAAIERETG
FQCSAGISHNKVLAKLACGLNKPNRQTLVSHGSVPQLFSQMPIRKIRSLGGKLGASVIEILGIEYMGELTQF
TESQLQSHFGEKNGSWLYAMCRGIEHDPVKPRQLPKTIGCSKNFPGKTALATREQVQWWLLQLAQELEE
RLTKDRNDNDRVATQLVVSIRVQGDKRLSSLRRCCALTRYDAHKMSHDAFTVIKNCNTSGIQTEWSPPLT
MLFLCATKFSASAPSSSTDITSFLSSDPSSLPKVPVTSSEAKTQGSGPAVTATKKATTSLESFFQKAAERQKV
KEASLSSLTAPTQAPMSNSPSKPSLPFQTSQSTGTEPFFKQKSLLLKQKQLNNSSVSSPQQNPWSNCKALP
NSLPTEYPGCVPVCEGVSKLEESSKATPAEMDLAHNSQSMHASSASKSVLEVTQKATPNPSLLAAEDQVP
CEKCGSLVPVWDMPEHMDYHFALELQKSFLQPHSSNPQVVSAVSHQGKRNPKSPLACTNKRPRPEGMQT
LESFFKPLTH,
SpCas9-D10A + H840A, dead Cas9, dCas9, without N-terminal
starting Methionine (M)
SEQ ID NO: 37
DKKYSIGLAIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMIEERLKTYA
HLEDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKEDNLTKAERGGLSELDKAGFIK
RQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLINLGAPAAFKYFDTTIDRKRYTSTKEV
LDATLIHQSITGLYETRIDLSQLGGD,
nCas12imax (SiCas12i-N243R + E336R), with N-terminal starting
Methionine (M)
SEQ ID NO: 38
MSSDVVRPYNTKLLPDNRKHNMFLQTFKRLNSISLNHFDLLICLYAAITNKKAEEYKSEKEAHVTADSLC
AINWFRPMSKRYSKYATTTFNMLELFKEYSGHEPDAYSKNYLMSNIDSDRFVWVDCRKFAKDFAYQMEL
GFHEFTVLAETLLANSILVLNESTKANWAWGTVSALYGGGDKEDSTLKSKILLAFVDALNNHELKTKREI
LNQVCESLKYQSYQDMYVDFRSVVDENGNKKSPRGSMPIVTKFETDDLISDNQRKAMISNETKNAAAK
AAKKPIPYLDRLKEHMVSLCDEYNVYAWAAAITNSNADVTARNTRNLTFIGEQNSRRKRLSVLQTTTNE
KAKDILNKINDNLIQEVRYTPAPKHLGRDLANLFDTLKEKDINNIENEEEKQNVINDCIEQYVDDCRSLNR
NPIAALLKHISRYYEDFSAKNFLDGAKLNVLTEVVNRQKAHPTIWSEKAYTWISKEDKNRRQANSSLVG
WVVPPEEVHKEKIAGQQSMMWVTLTLLDDGKWVKHHIPFSDSRYYSEVYAYNPNLPYLDGGIPRQSKFG
NKPTTNLTAESQALLANSKYKKANKSFLRAKENATHNVRVSPNTSLCIRLLKDSAGNQMFDKIGNVLFG
MQINHKITVGKPNYKIEVGDRFLGFDQNQSENHTYAVLQRVSESSHDTHHENGWDVKVLEKGKVTSDVI
VRDEVYDQLSYEGVPYDSSKFAEWRDKRRRFVLENLSIQLEEGKTFLTEFDKLNKDSLYRWNMNYLKLL
RKAIRAGGKEFAKIAKTEIFELAVERFGPINLGSLSQISLKMIASFKGVVQSYESVSGCVDDASKKAHDSM
LFTFMCAAEEKRTNKREEKTNRAASFILQKAYLHGCKMIVCEDDLPVADGKTGKAQNADRMDRCARAL
AKKVNDGCVAMSICYRAIPAYMSSHQDPFVHMQDKKTSVLRPRFMEVNKDSIRDYHVAGLRRMLNSKS
DAGTSVYYRQAALHFCEALGVSPELVKNKKTHAAELGKHMGSAMLMPWRGGRVYIASKKLTSDAKSV
KYCGEDMWQYHADEIAAVNIAMYEVCCQTGAFGKKQKKSDELPG,
scaffold sequence (direct repeat (DR) sequence) for nCas12imax
SEQ ID NO: 39
AGAAATGTGTCCCCAGTTGACAC,

TABLE A
Homo sapiens; Pan troglodytes; Pongo abelii; Theropithecus gelada; Gorilla gorilla gorilla; Cercocebus atys;
Mandrillus leucophaeus; Hylobates moloch; Macaca nemestrina; Rhinopithecus bieti; Chlorocebus sabaeus;
Sapajus apella; Callithrix jacchus; Aotus nancymaae; Cebus imitator; Propithecus coquereli; Otolemur
garnettii; Microcebus murinus; Ursus arctos; Ailuropoda melanoleuca; Molossus molossus; Equus caballus;
Hyaena hyaena; Panthera leo; Canis lupus familiaris; Castor canadensis; Puma concolor; Sciurus carolinensis;
Panthera tigris; Equus przewalskii; Canis lupus dingo; Hipposideros armiger; Puma yagouaroundi; Panthera
uncia; Suricata suricatta; Felis catus; Rangifer tarandus platyrhyncus; Ovis aries; Moschus berezovskii;
Miniopterus natalensis; Phacochoerus africanus; Rousettus aegyptiacus; Marmota marmota marmota;
Galeopterus variegatus; Marmota monax; Tupaia chinensis; Lynx pardinus; Odocoileus virginianus texanus;
Cervus elaphus; Meles meles; Myotis lucifugus; Sorex araneus; Bos mutus; Arvicola amphibius; Suncus
etruscus; Pteronotus parnellii mesoamericanus; Capra hircus; Nycticebus coucang; Myotis brandtii; Chinchilla
lanigera; Enhydra lutris kenyoni; Diceros bicornis minor; Heterocephalus glaber; Apodemus sylvaticus;
Vicugna pacos; Mus musculus; Mastomys coucha; Arvicanthis niloticus; Octodon degus; Cricetulus griseus;
Peromyscus leucopus; Acomys russatus; Eptesicus fuscus; Sus scrofa; Mustela putorius furo; Grammomys
surdaster; Eschrichtius robustus; Manis pentadactyla; Camelus ferus; Camelus bactrianus; Mesocricetus
auratus; Rattus rattus; Dipodomys spectabilis; Camelus dromedarius; Onychomys torridus; Choloepus
didactylus; Fukomys damarensis; Nannospalax galili; Oryctolagus cuniculus; Perognathus longimembris
pacificus; Lutra lutra; Orycteropus afer afer; Cavia porcellus; Manis javanica; Phodopus roborovskii; Mirounga
angustirostris; Dipodomys ordii; Talpa occidentalis; Pipistrellus kuhlii; Ochotona curzoniae; Antechinus
flavipes; Trichechus manatus latirostris; Sarcophilus harrisii; Trichosurus vulpecula; Phascolarctos cinereus;
Loxodonta africana; Gracilinanus agilis; Vombatus ursinus; Monodelphis domestica; Chrysochloris asiatica;
Meriones unguiculatus; Dromiciops gliroides; Echinops telfairi; Elephantulus edwardii; Rhinolophus sinicus;
Chrysemys picta bellii; Dermochelys coriacea; Malaclemys terrapin pileata; Trachemys scripta elegans;
Gopherus evgoodei; Chelonoidis abingdonii; Caretta caretta; Mauremys mutica; Mauremys reevesii; Apteryx
mantelli mantelli, Centropus unirufus; Nyctereutes procyonoides; Tachyglossus aculeatus; Alligator
mississippiensis; Acipenser ruthenus; Tympanuchus pallidicinctus; Pelodiscus sinensis; Centrocercus
urophasianus; Geotrypetes seraphini; Nipponia nippon; Latimeria chalumnae; Microcaecilia unicolor;
Meleagris gallopavo; Lagopus muta; Hirundo rustica rustica; Strigops habroptila; Coturnix japonica; Zosterops
borbonicus; Motacilla alba alba; Gavia stellata; Spizaetus tyrannus; Lamprotornis superbus; Apus apus;
Onychostruthus taczanowskii; Bufo bufo; Burhinus bistriatus; Lonchura striata domestica; Engystomops
pustulosus; Bufo gargarizans; Chloebia gouldiae; Rana temporaria; Scyliorhinus torazame; Brienomyrus
brachyistius; Xenopus tropicalis; Salvelinus alpinus; Carcharodon carcharias; Nanorana parkeri; Oncorhynchus
nerka; Pitangus sulphuratus; Podarcis muralis; Varanus komodoensis; Xenopus laevis; Thunnus albacares;
Bombina bombina; Protopterus annectens; Polypterus senegalus; Puntigrus tetrazona; Scomber japonicus;
Synchiropus splendidus; Clarias gariepinus; Megalops cyprinoides; Plectropomus leopardus; Chanos chanos;
Zootoca vivipara; Lacerta agilis; Cheilinus undulatus; Alosa alosa; Eublepharis macularius; Sinocyclocheilus
grahami; Tachysurus fulvidraco; Micropterus dolomieu; Lerista edwardsae; Paramormyrops kingsleyae;
Synaphobranchus kaupii; Cottoperca gobio; Colossoma macropomum; Siniperca chuatsi; Pangasius djambal;
Lates calcarifer; Bagarius yarrelli; Silurus meridionalis; Oryzias melastigma; Myripristis murdjan; Ictalurus
punctatus; Syngnathus scovelli; Gymnodraco acuticeps; Oryzias javanicus; Tetraodon nigroviridis;
Larimichthys crocea; Gymnothorax javanicus; Pangasianodon gigas; Epinephelus fuscoguttatus; Ictalurus
furcatus; Sphaerodactylus townsendi; Notechis scutatus; Monopterus albus; Rattus norvegicus; Labeo rohita;
Pristis pectinata; Danionella translucida; Betta splendens; Scleropages formosus; Denticeps clupeoides;
Hymenochirus boettgeri; Pantherophis guttatus; Acanthopagrus latus; Thalassophryne amazonica; Nibea
albiflora; Epinephelus lanceolatus; Phrynocephalus forsythii; Protobothrops mucrosquamatus; Cynoglossus
semilaevis; Hemicordylus capensis; Sceloporus undulatus; Spea bombifrons; Sparus aurata; Thamnophis
elegans; Crotalus tigris; Amblyraja radiata; Perca fluviatilis; Perca flavescens; Danio rerio; Rissa tridactyla;
Naja naja; Labrus bergylta; Takifugu rubripes; Toxotes jaculatrix; Sander lucioperca; Etheostoma spectabile;
Etheostoma cragini; Callipepla squamata; Anguilla anguilla; Mastacembelus armatus; Stegastes partitus;
Pseudonaja textilis; Electrophorus electricus; Pleurodeles waltl; Austrofundulus limnaeus; Conger conger;
Pleuronectes platessa; Pogona vitticeps; Callorhinchus milli; Nothobranchius furzeri; Oryzias latipes; Gadus
morhua; Scophthalmus maximus; Polyodon spathula; Araneus ventricosus; Anabarilius grahami; Anolis
carolinensis; Gekko japonicus; Dicentrarchus labrax; Clupea harengus; Lepisosteus oculatus; Notolabrus
celidotus; Takifugu flavidus; Nematolebias whitei; Uloborus diversus; Python bivittatus; Petromyzon marinus;
Asterias rubens; Seriola lalandi dorsalis; Sinocyclocheilus anshuiensis; Trichonephila clavata; Phocoena sinus;
Nephila pilipes; Trichonephila inaurata madagascariensis; Stegodyphus dumicola; Stegodyphus mimosarum;
Caerostris darwini; Saccoglossus kowalevskii; Lingula anatina; Branchiostoma floridae; Procambarus clarkii;
Cinara cedri; Penaeus monodon; Sipha flava; Crassostrea gigas; Penaeus vannamei; Crassostrea virginica;
Dreissena polymorpha; Portunus trituberculatus; Aplysia californica; Daktulosphaira vitifoliae; Zootermopsis
nevadensis; Holothuria leucospilota; Centruroides sculpturatus; Branchiostoma belcheri; Biomphalaria
glabrata; Pecten maximus; Apostichopus japonicus; Capitella teleta; Polistes dominula; Argiope bruennichi;
Oedothorax gibbosus; Owenia fusiformis; Exaiptasia diaphana; Ignelater luminosus; Vespula vulgaris; Haliotis
rufescens; Fopius arisanus; Aphis glycines; Rhopalosiphum maidis; Polistes canadensis; Mizuhopecten
yessoensis; Acropora millepora; Anneissia japonica; Coptotermes formosanus; Vespa crabro; Elysia chlorotica;
Parasteatoda tepidariorum; Bulinus truncatus; Medioppia subpectinata; Branchiostoma lanceolatum; Aphis
gossypii; Melanaphis sacchari; Zophobas morio; Gigantopelta aegis; Aphis craceivora; Soboliphyme baturini;
Cryptotermes secundus; Ixodes scapularis; Diachasma alloeum; Solenopsis invicta; Orbicella faveolata; Idotea
baltica; Lamprigera yunnana; Pomacea canaliculata; Haliotis rubra; Cephus cinctus; Stylophora pistillata;
Pogonomyrmex barbatus; Chelonus insularis; Nematostella vectensis; Coccinella septempunctata; Leptopilina
heterotoma; Homarus americanus; Cotesia congregata; Nezara viridula; Myzus persicae; Trichogramma
pretiosum; Nasonia vitripennis; Harmonia axyridis; Lamellibrachia satsuma; Tenebrio molitor; Desmophyllum
pertusum; Halyomorpha halys; Adelges cooleyi; Ctenocephalides felis; Cotesia typhae; Agrilus planipennis;
Dermacentor andersoni; Acyrthosiphon pisum; Planctomycetota bacterium; Ciona intestinalis; Osmia lignaria;
Neogale vison; Monomorium pharaonis; Patiria miniata; Osmia bicornis bicornis; Thioalkalivibrio sp. XN279;
Linepithema humile; Dendronephthya gigantea; Pocillopora damicornis; Acanthoscelides obtectus; Ostrea
edulis; Orussus abietinus; Thioalkalivibrio sp. XN8; Trichoplax sp. H2; Apis laboriosa; Trichoplax adhaerens;
Pediculus humanus corporis; Megachile rotundata; Acromyrmex charruanus; Temnothorax curvispinosus;
Eurytemora affinis; Frieseomelitta varia; Hypsibius exemplaris; Cotesia glomerata; Copidosoma floridanum;
Aethina tumida; Trichinella zimbabwensis; Armadillidium vulgare; Venturia canescens; Ooceraea biroi;
Wasmannia auropunctata; Trichinella spiralis; Octopus sinensis; Acromyrmex echinatior; Cyphomyrmex
costatus; Ceratina calcarata; Rhipicephalus sanguineus; Batillaria attramentaria; Dufourea novaeangliae;
Formica exsecta; Callosobruchus maculatus; Ixodes persulcatus; Hyalella azteca; Nomia melanderi; Lytechinus
variegatus; Psylliodes chrysocephala; Daphnia pulicaria; Gonioctena quinquepunctata; Microplitis demolitor;
Lytechinus pictus; Bemisia tabaci; Leptotrombidium deliense; Tribolium castaneum; Actinia tenebrosa;
Acanthaster planci; Haemaphysalis longicornis; Eufriesea mexicana; Cimex lectularius; Belonocnema kinseyi;
Daphnia pulex; Rhipicephalus microplus; Bombus pyrosoma; Terrapene carolina triunguis; Diuraphis noxia;
Trachymyrmex septentrionalis; Wenzhouxiangella sp. XN24; Dinothrombium tinctorium; Timema monikensis;
Phycisphaerales bacterium; Ceratosolen solmsi marchali; Trachymyrmex cornetzi; Timema californicum;
Eriocheir sinensis; Cloeon dipterum; Neodiprion pinetum; Xenia sp. Carnegie-2017; Gryllus bimaculatus;
Neodiprion virginianus; Phyllotreta striolata; Timema poppensis; Trichuris suis; Diprion similis; Mya arenaria;
Homalodisca vitripennis; Timema cristinae; Holotrichia oblita; Strongylocentrotus purpuratus; Apolygus
lucorum; Cataglyphis hispanica; Plakobranchus ocellatus; Timema tahoe; Timema douglasi; Hylaeus
anthracinus; Pseudomyrmex gracilis; Ampulex compressa; Leptinotarsa decemlineata; Notodromas monacha;
Trichuris trichiura; Anoplophora glabripennis; Tribolium madens; Periplaneta americana; Diabrotica virgifera
virgifera; Sitophilus oryzae; Atta colombica; Darwinula stevensoni; Phaedon cochleariae; Thrips palmi;
Cyanobacteria bacterium CYA; Schistocerca gregaria; Vollenhovia emeryi; Schistocerca serialis cubense;
Patella vulgata; Priapulus caudatus; Macrosteles quadrilineatus; Planctomycetales bacterium ZRK34;
Tropilaelaps mercedesae; Acidobacteriota bacterium; Bombus terrestris; Oppia nitens; Leptopilina boulardi;
Scortum barcoo; Bombus vosnesenskii; Phallusia mammillata; Dinoponera quadriceps; Phycisphaeraceae
bacterium; Ischnura elegans; Diaphorina citri; Eretmocerus hayati; Blattella germanica; Chrysoperla carnea;
Photinus pyralis; Oopsacas minuta; Diabrotica balteata; Armadillidium nasatum; Trinorchestia longiramus;
Mytilus galloprovincialis; Schistocerca nitens; Aphidius gifuensis; Nicrophorus vespilloides; Frankliniella
occidentalis; Gammaproteobacteria bacterium; Brassicogethes aeneus; Encephalitozoon hellem; Phycisphaerae
bacterium; Amphimedon queenslandica; Harpegnathos saltator; Mytilus coruscus; Vittaforma corneae ATCC
50505; Encephalitozoon romaleae SJ-2008; Galendromus occidentalis; Ramazzottius varieomatus; Daphnia
magna; Athalia rosae; Daphnia sinensis; Ladona fulva; Encephalitozoon cuniculi GB-MI; Callosobruchus
chinensis; Cyprinus carpio; Trachymyrmex zeteki; Fusobacterium necrogenes; Encephalitozoon intestinalis
ATCC 50506; Phycisphaera sp.; Tigriopus californicus; Brachionus calyciflorus; Onthophagus taurus;
Halotydeus destructor; Anaerolineae bacterium; Oppiella nova; Planctomycetaceae bacterium; Bradysia
odoriphaga; Planctomycetales bacterium; Camponotus floridanus; Propionispora vibrioides; Dimorphilus
gyrociliatus; Sarcoptes scabiei; Folsomia candida; Deltaproteobacteria bacterium RIFOXYA12_FULL_58_15;
Dermatophagoides farinae; Candidatus Parcubacteria bacterium; Methanocella sp. CWC-04; Leptolyngbya sp.
PLA1; Clostridium polyendosporum; bacterium; Rickettsiales bacterium; Nilaparvata lugens; Clostridium
perfringens; Asbolus verrucosus; Abscondita terminalis; Clostridium saudiense; Odontomachus brunneus;
Tepidimicrobium xylanilyticum; Megalopta genalis; Clostridium sp. DJ247; Abditibacteriota bacterium;
Candidatus Desantisbacteria bacterium CG1_02_38_46; Clostridium acidisoli; Rubeoparvulum massiliense;
Clostridium estertheticum; Chloroflexota bacterium; Gemmatimonadota bacterium; Colletes gigas; Clostridium
sp. CS001; Apis florea; Clostridium sp. Cult1; Propionigenium maris DSM 9537; Deltaproteobacteria
bacterium RIFCSPLOWO2_12_FULL_60_19; Menidia menidia; Caldilineae bacterium; Clostridium sp. CX1;
Methanocella conradii; Dermatophagoides pteronyssinus; Sporohalobacter salinus; Leptolyngbya sp.;
Muricomes intestini; Tetranychus urticae; Propionispora hippei; Planctomycetes bacterium Poly30; Candidatus
Portnoybacteria bacterium RIFCSPLOWO2 01 FULL 43 11; Clostridium manihotivorum;
Deltaproteobacteria bacterium; Candidatus Buchananbacteria bacterium RBG 13 39 9; Acidobacteria
bacterium 13_1_40CM_56_16; Propionispora hippei DSM 15287; uncultured Clostridium sp.; Apis dorsata;
Candidatus Portnoybacteria bacterium CG03_land_8_20_14_0_80_41_10; Paenibacillus dendritiformis;
Clostridium sp. C8-1-8; Bacillota bacterium; Tissierellia bacterium; Clostridium collagenovorans; Nitrospinota
bacterium; Candidatus Bathyarchaeota archaeon; Candidatus Nealsonbacteria bacterium; Paenibacillus
rhizolycopersici; Atta cephalotes; Candidatus Portnoybacteria bacterium; Methanocella arvoryzae; unclassified
Clostridium; Caloramator proteoclasticus; Pyrinomonadaceae bacterium; Methanocella paludicola; Candidatus
Binatia bacterium; Caloramator fervidus; Habropoda laboriosa; Panonychus citri; Patescibacteria group
bacterium; Propionispora sp. 2/2-37; Paenibacillus sp. ACRRX; Parcubacteria group bacterium; Clostridiales
bacterium; Clostridium tetani; Caerostris extrusa; Fusobacterium perfoetens; Clostridium tagluense;
Selenihalanaerobacter shriftii; Paenibacillus phocaensis; Anaeromicropila populeti; Caloramator sp.;
Clostridium; Eubacteriaceae bacterium; Acetohalobium arabaticum; unclassified Psychrilyobacter;
Psychrilyobacter sp. BL5; Candidatus Uhrbacteria bacterium; Sinanaerobacter chloroacetimidivorans; Blomia
tropicalis; Clostridium aestuarii; Orenia metallireducens; Clostridium botulinum; Clostridium liquoris;
Tepidisphaera sp.; Pseudomonadota bacterium; Candidimonas sp.; Parcubacteria group bacterium
RIFCSPHIGHO2_02_FULL_48_10b; Caloramator australicus; Clostridium cochlearium; candidate division
NC10 bacterium; Clostridium thailandense; Clostridium sp. C105KSO13; Caloramator sp. Dgby_cultured_2;
Eciton burchellii; Paenibacillus taiwanensis; Syntrophobotulus glycolicus; uncultured bacterium;
Natranaerobius thermophilus; Oxobacter pfennigii; Clostridium sp. D1-1; Anaerolineales bacterium;
Heterotrigona itama; Paenibacillus barengoltzii; Vulgatibacter incomptus; Paenibacillus sp. HMSSN-139;
Hypnocyclicus thermotrophus; Candidatus Nealsonbacteria bacterium
CG11_big_fil_rev_8_21_14_0_20_37_68; Ruminiclostridium cellobioparum; Clostridia bacterium;
Clostridium sp. Marseille-Q2269; Clostridium prolinivorans; Clostridium fungisolvens; Clostridium
yunnanense; Desulfitobacterium chlororespirans; Clostridium oryzae; Desulfitobacterium chlororespirans DSM
11544; Vespula germanica; Peptoniphilus phoceensis; Gottschalkia acidurici; Didymodactylos carnosus;
Verrucomicrobiales bacterium; Geotoga petraea; Parapusillimonas sp. SGNA-6; Polyangiaceae bacterium;
Alkaliphilus pronyensis; Ardenticatenia bacterium; Pusillimonas harenae; Pusillimonas soli; Ruminiclostridium
herbifermentans; Gemmatimonadales bacterium; Candidatus Latescibacteria bacterium ADurb.Bin168;
Castellaniella denitrificans; Paenibacillus anaericanus; Clostridium akagii; Pusillimonas sp. T7-7;
Desulfosporosinus sp. FKB; Eleutherodactylus coqui; Paenibacillus sp. MER TA 81-3; Thermoflexales
bacterium; Allacma fusca; Clostridium tepidum; Clostridium lundense; Desulfitobacterium dehalogenans;
Clostridium sp. JN-1; Kiritimatiellia bacterium; candidate division Zixibacteria bacterium; Gottschalkia
purinilytica; Clostridium rhizosphaerae; Polistes exclamans; Schnuerera ultunensis; Desulfosporosinus
youngiae; Verrucomicrobiota bacterium; Noviherbaspirillum aridicola; Pusillimonas ginsengisoli; Natronincola
ferrireducens; Culicoidibacter larvae; Lutispora thermophila; Clostridium cellulovorans; Parcubacteria
bacterium DG 74 3; Pusillimonas sp. M17; Moraxellaceae bacterium; Paenibacillus sp. 481;
Gemmatimonadetes bacterium 13 1 40CM 3 69 22; Thermobrachium celere; Clostridium aminobutyricum;
Candidatus Rokubacteria bacterium; Halanaerobium saccharolyticum; Parcubacteria group bacterium
Gr01-1014_38; Clostridium frigoris; Acanthopleuribacter pedis; Clostridium sporogenes; Clostridium mobile;
Paenibacillus sp. SC116; Oscillospiraceae bacterium; Candidatus Marinimicrobia bacterium; Paenibacillus
arenosi; Paenibacillus sp. FSL R7-0273; Sulfobacillus benefaciens; Clostridium septicum; Apis cerana cerana;
Oceanirhabdus sp. W0125-5; Sphaerochaeta coccoides; Clostridium sp.; Desulfosporosinus orientis;
Candidatus Portnoybacteria bacterium RBG_13_40_8; Vicinamibacterales bacterium; Tissierellales;
unclassified Paenibacillus; Sphingobacteriia bacterium; Gelria sp. Kuro-4; Candidatus Odinarchaeota archaeon;
Anaerolineaceae bacterium; Burkholderiales bacterium; Thermoflexia bacterium; Clostridium caldaquaticum;
Synergistaceae bacterium; Crassaminicella thermophila; Ignavibacteriota bacterium; Epulopiscium sp.;
Actinomycetia bacterium; Microgenomates group bacterium LiPW_16; Clostridium muellerianum;
Alcaligenaceae; Firmicutes bacterium HGW-Firmicutes-13; Methanobacterium; Limosa lapponica baueri;
Keratinibaculum paraultunense; Paenibacillus profundus; Tissierellaceae bacterium; Faecalicatena contorta;
Pusillimonas sp. TS35; Paenibacillus sp. J53TS2; Clostridium sp. BL-8; Clostridium hydrogenum; Clostridium
butyricum; Blastocatellia bacterium; Anaerococcus vaginalis; Aneurinibacillus aneurinilyticus; Clostridium
tunisiense; Paenibacillus apiarius; Acidobacteria bacterium ADurb.Bin051; Legionella sp.;; Candidatus
Niyogibacteria bacterium CG10_big_fil_rev_8_21_14_0_10_46_36; Garciella nitratireducens; Halobacteroides
halobius; Alkaliphilus flagellatus; Alcaligenaceae bacterium; Candidatus Aminicenantes bacterium; Orenia
marismortui; Alkaliphilus peptidifermentans; Paenibacillus tengchongensis; Clostridium chauvoei;
Fusobacteria bacterium ZRK30; Candidatus Nealsonbacteria bacterium CG 4 10 14 0 2 um filter 35 20;
Chromatiales bacterium 21-64-14; Alkaliphilus transvaalensis; Myxococcales bacterium; Candidatus
Eremiobacteraeota bacterium; Clostridium cadaveris; Anaerococcus nagyae; Oxalobacteraceae bacterium OMI;
Peptoniphilus stercorisuis; Clostridium saccharobutylicum; Candidatus Egerieicola faecale; Paenibacillus
macerans; Clostridium baratii; Sporomusaceae bacterium; Candidatus Omnitrophica bacterium 4484_49;
Aneurinibacillus terranovensis; Clostridium sp. 29_15; Burkholderiaceae bacterium; Castellaniella defragrans;
Anaerococcus senegalensis; Gemmatimonadaceae bacterium; Mucisphaera calidilacus; Candidatus
Calescamantes bacterium; Methanocella sp. PtaU1.Bin125; Mischocyttarus mexicanus; Castellaniella
defragrans 65Phen; Alkaliphilus oremlandii; Haloimpatiens massiliensis; Clostridium sp. LS; Algisphaera
agarilytica; Candidatus Egerieicola pullicola; Clostridium acetobutylicum; Salicibibacter kimchii; Candidatus
Uhrbacteria bacterium RIFCSPHIGHO2_02_FULL_46_47; Paenibacillus agilis; Alcaligenaceae bacterium
CGII-47; Actinomycetota bacterium; Clostridium sp. TW13; Clostridium rectalis; Candidatus
Buchananbacteria bacterium; Bryobacterales bacterium; Clostridium sp. CAG: 221; Paenibacillus sp. P32E;
Clostridium sp. SM-530-WT-3G; Pseudobdellovibrionaceae bacterium; Galdieria sulphuraria;
Chloracidobacterium sp.; Desulfitobacterium sp. PCE1; Natribacillus halophilus; Ruminiclostridium hungatei;
Candidatus Niyogibacteria bacterium RIFCSPLOWO2_12_FULL_41_13; Castellaniella sp.; Clostridium
bowmanii; Paenibacillus senegalimassiliensis; Oscillibacter sp.; Paenibacillus auburnensis; Clostridium
taeniosporum; Paracandidimonas soli; Halonatronum saccharophilum; Candidatus Poribacteria bacterium;
Fusobacterium sp.; Sorangium cellulosum; Haloplasma contractile; Clostridium tetanomorphum;
Ignavibacteriales bacterium; Clostridium cavendishii; Tepiditoga spiralis; Geomonas silvestris;
Limnochordaceae bacterium; Clostridium sp. C2-6-12; Oceanotoga sp. DSM 15011; Gemmatimonadetes
bacterium 13_1_40CM_4_69_8; Clostridium fallax; Paenibacillus piscarius; Pseudalkalibacillus berkeleyi;
Pusillimonas sp. MFBS29; Orchesella cincta; Paenibacillus assamensis; Holophagales bacterium; Thiolapillus
brandeum; Phycisphaera mikurensis; Romboutsia sp. CE17; Anaeromicrobium sediminis; Parapusillimonas
granuli; Romboutsia; Paenibacillus segetis; Bacteroidota bacterium; Clostridium mediterraneense;
Halanaerobium sp. ST460_2HS_T2; Clostridium pasteurianum; Paenibacillus sp. P3E; Elusimicrobiota
bacterium; Clostridium hydrogeniformans; Geomicrobium sp. JCM 19055; Paenibacillus oryzae; Tissierella
simiarum; Candidatus Omnitrophota bacterium; Oscillibacter valericigenes; Halanaerobium kushneri;
Candidatus Niyogibacteria bacterium RIFCSPLOWO2_02_FULL_45_13; Xanthomonadales bacterium;
Sedimentisphaerales bacterium; Lentisphaerae bacterium GWF2_57_35; Candidatus Cellulosilyticum
pullistercoris; Candidatus Lokiarchaeota archaeon; Paenibacillus silagei; Geomonas limicola; Desnuesiella
massiliensis; Nitrospirota bacterium; Paenibacillus camerounensis; Clostridium sp. N3C; Candidatus
Niyogibacteria bacterium; Ruminiclostridium sufflavum; Acidobacteriaceae bacterium; Clostridium
carboxidivorans; Paenibacillus riograndensis; Clostridium gelidum; Pusillimonas noertemannii; Hortaea
werneckii; Paenibacillus sp. Marseille-P2973; Caldisericota bacterium; Marasmitruncus massiliensis; Aquicella
siphonis; Flavobacteriaceae bacterium CRH; Ignavibacteriaceae bacterium; Sporomusaceae bacterium FL31;
Niameybacter massiliensis; Willisornis vidua; Deltaproteobacteria bacterium GWC2 65 14; Sulfurifustis
variabilis; Hathewaya massiliensis; Betaproteobacteria bacterium GR16-43; Caloramator mitchellensis;
Poriferisphaera corsica; Paenibacillus sonchi; Herpetosiphon sp.; Cellulosilyticum sp. WCF-2; Peptoniphilus
timonensis; candidate division WWE3 bacterium CG06 land 8 20 14 3 00 42 16; Clostridium sp. DSM
17811; Candidatus Cloacimonadota bacterium; Saccharibacillus brassicae; Peptoniphilus sp. HMSC062D09;
Usitatibacter rugosus; Bdellovibrio sp.; Desulfotruncus arcticus; Paenibacillus sp. DMB5; Myxococcaceae
bacterium; Candidatus Kryptonium thompsoni; Paenibacillus sp. PK3 47; Crassaminicella profunda;
Ignavibacteriales bacterium CG07_land_8_20_14_0_80_59_12; Caldilineales bacterium; Sulfidibacter
corallicola; Paenibacillus tianjinensis; Pollutimonas subterranea; Herbinix luporum; Ignavibacteria bacterium;
Betaproteobacteria bacterium; Aneurinibacillus migulanus; Clostridium senegalense; Gemmatimonadetes
bacterium 13_1_20CM_4_69_16; Clostridium sulfidigenes; Candidatus Kryptobacter tengchongensis;
Galdieria partita; Paeniclostridium sordellii; Desulfosporosinus sp. BRH_c37; Thermoanaerobaculia bacterium;
Dethiobacter alkaliphilus; Gemmatimonas sp. SG8_28; Paenibacillus borealis; Saccharibacillus sp. WB 17;
Paenibacillus faecalis; Clostridium felsineum; Zhenhengia yiwuensis; Parcubacteria group bacterium
ADurb.Bin159; Rubrivivax sp.; Brockia lithotrophica; Paeniclostridium hominis; Saprospiraceae bacterium;
Solimonas fluminis; Clostridium bovifaecis; Candidatus Woesearchaeota archaeon; Portibacter marinus;
Paenibacillus sp. FSL R5-0912; Paenibacillus tepidiphilus; unclassified Candidatus Frackibacter; Solimonas sp.
K1W22B-7; Chlorobium sp.; Clostridium ljungdahlii; Paenibacillus woosongensis; Pedobacter sp.; Hathewaya
histolytica; Akkermansiaceae bacterium; Tyrophagus putrescentiae; Alkaliphilus sp.; Sebaldella sp.;
Clostridiaceae bacterium HFYG-1003; Prosthecochloris; Gemmatales bacterium; Treponema sp.; Candidatus
Eisenbacteria bacterium; Paenibacillus typhae; Paenibacillus graminis; Candidatus Moranbacteria bacterium
GW2011_GWF1_34_10; Anaerosolibacter carboniphilus; Halanaerobium sp.; Clostridium novyi;
Mesorhizobium sp. ES1-1; Halanaerobium congolense; Clostridium frigidicarnis; Steroidobacter
agaridevorans; Pedobacter mucosus; Clostridium luticellarii; Xanthomonadaceae bacterium; Pullulanibacillus
pueri; Steroidobacter gossypii; Lachnospiraceae bacterium NSJ-143; Legionella erythra; Candidatus Sumerlaea
chitinivorans; Tetrahymena thermophila SB210; Candidatus Kapabacteria bacterium; Candidatus Azambacteria
bacterium RBG_16_47_10; Noviherbaspirillum aerium; Rhodospirillales bacterium; Candidatus Moranbacteria
bacterium GW2011_GWE1_35_17; Aridibacter famidurans; Paenibacillus shirakamiensis; Ardenticatenales
bacterium; Clostridium colicanis; Natroniella sulfidigena; Legionella adelaidensis; Candidatus
Thermoplasmatota archaeon; Lutibacter sp. B2; Oncorhynchus mykiss; bacterium (Candidatus
Gribaldobacteria) CG02_land_8_20_14_3_00_41_15; Candidatus Hydrogenedentes bacterium; Pelodictyon
luteolum; Rhodanobacter sp.; Bacillus luti; Paenibacillus dakarensis; Clostridium butyricum 60E.3;
Cellulosilyticum sp.; Desulfobacterales bacterium; Halobacteriales archaeon; Chlorobiaceae bacterium;
candidate division BRC1 bacterium SM23_51; Calditrichota bacterium; Fusobacterium varium; Candidimonas
humi; Acetobacteraceae bacterium; candidate division Zixibacteria bacterium SM1_73; Geobacter sp. DSM
9736; Legionella yabuuchiae; Limnochordales bacterium; Janthinobacterium sp. 17J80-10; Syntrophaceae
bacterium; Pedobacter sp. SYSU D00535; uncultured Acidobacteriota bacterium; Herpetosiphon geysericola;
Paenibacillus tritici; Leptospirillum ferriphilum; Candidatus Heimdallarchaeota archaeon; Nevskia sp.;
Mesorhizobium sp. AR10; Beijerinckia sp. 28-YEA-48; Bdellovibrionales bacterium; Bacillus cereus;
bacterium HR10; Clarias magur; Clostridium sp. Marseille-P2415; Chlorobaculum thiosulfatiphilum;
Candidatus Saccharicenans sp.; Candidatus Manganitrophus noduliformans; Verrucomicrobia subdivision 3
bacterium; Desulfobacteraceae bacterium; Peptoniphilus sp. HMSC075B08; Peptoniphilus gorbachii;
Acetivibrio saccincola; Paenibacillus sp. S150; Parcubacteria group bacterium CG2_30_44_18;
Psychrilyobacter atlanticus; Lacrimispora saccharolytica; Aquisphaera sp. JC669; Singulisphaera acidiphila;
unclassified Romboutsia; Chitinophagia bacterium; Gammaproteobacteria bacterium
RIFCSPHIGHO2_12_FULL_41_20; Anaerococcus sp. mt242; Paenibacillus caui; Nevskia soli; Sorangium;
Paenibacillus ihumii; Lysobacter telluris; Acholeplasmataceae bacterium; Peptoniphilus sp. SAHP1;
Bradymonadaceae bacterium; Paenibacillus sp. FSL P4-0081; Aneurinibacillus sp. Ricciae_BoGa-3;
Peptoniphilus senegalensis; Bacteroidia bacterium; Clostridium sp. C8; Lachnospiraceae bacterium;
Acidobacteriia bacterium; Verrucomicrobiae bacterium; Castellaniella sp. S9; Candidatus Angelobacter sp.;
Saprospirales bacterium; Gammaproteobacteria bacterium RIFCSPHIGHO2_12_FULL_37_34;
Deltaproteobacteria bacterium CG2_30_66_27; Pedobacter frigidisoli; Clostridium algifaecis; Candidatus
Buchananbacteria bacterium RBG_13_36_9; Sporanaerobacter acetigenes; Pedobacter sp. Leaf176;
Parcubacteria group bacterium GW2011_GWA2_42_80; Mesorhizobium sp. B2-3-3; Fictibacillus
phosphorivorans; Paenibacillus vini; Parcubacteria group bacterium GW2011_GWF2_43_11;
Noviherbaspirillum malthae; Clostridium sp. ‘White wine YQ’; Deltaproteobacteria bacterium GWA2 65 63;
Pollutimonas nitritireducens; Deltaproteobacteria bacterium CG_4_9_14_3_um_filter_65_9; Chryseobacterium
carnipullorum; Flavobacterium johnsoniae; Chloroflexi bacterium ADurb.Bin360; Nannochloropsis salina
CCMP1776; Chryseobacterium sp. CY350; Sorangium cellulosum So ce56; Candidatus Azambacteria
bacterium; Gammaproteobacteria bacterium RIFCSPHIGHO2 12 FULL 37 14; Melioribacteraceae
bacterium; Natroniella acetigena; Salicibibacter cibarius; Paeniclostridium ghonii; Flavobacterium; Penaeicola
halotolerans; Clostridium sartagoforme AAU1; Pullulanibacillus camelliae; Portibacter lacus;
Acidaminococcaceae bacterium; Candidatus Falkowbacteria bacterium; Crassaminicella indica;
Gammaproteobacteria bacterium RIFCSPHIGHO2_12_38_15; Herpetosiphon llansteffanensis; Rhabdobacter
roseus; Hydrogenibacillus schlegelii; Pedobacter sp. AK013; Methanomassiliicoccales archacon PtaU1.Bin030;
Silvibacterium sp.; Bacillus cereus group; Clostridium formicaceticum; Chryseobacterium sp. LJ668;
Clostridium nigeriense; Thermoprotei archaeon; Candidatus Manganitrophaceae bacterium; Pedobacter
borealis; Leptospirillum ferriphilum YSK; Aliifodinibius roseus; Pedobacter terrae; Paenibacillus hunanensis;
Turicibacter sp.; Deltaproteobacteria bacterium GWA2_45_12; Flavobacterium frigidimaris; Succiniclasticum
ruminis; Anaeromyxobacter dehalogenans; Actinobacteria bacterium RBG_19FT_COMBO_70_19; Legionella
jordanis; Candidatus Korarchaeota archaeon; Acidipila sp. EB88; Platysternon megacephalum; Pedobacter sp.
BMA; Tepidibacter thalassicus; Clostridiaceae bacterium; Clostridium aceticum; Halomonas sp.; Castellaniella
caeni; Clostridium sp. MSJ-8; Clostridium sp. D43t1_170807_H7; Halarsenatibacter silvermanii;
Aneurinibacillus sp. B1; Sulfobacillus sp. hq2; Leptospirillum sp. Group II ‘CF-1’; Microgenomates group
bacterium GW2011_GWA2_39_19; Clostridium culturomicium; Peptoniphilus tyrrelliae; Candidatus
Krumholzibacteriota bacterium; Fictibacillus macauensis; Paenibacillus sp. FSL R7-277; Legionella
longbeachae; Legionella sp. 27cVA30; Inconstantimicrobium porci; Paenibacillus fonticola;
Geomesophilobacter sediminis; Paenibacillus zeisoli; Mesorhizobium sp. LNJC394B00; Neofamilia
massiliensis; Solimicrobium sp.; Clostridium carboxidivorans P7; Legionella lansingensis; Parcubacteria group
bacterium Gr01-1014_106; Noviherbaspirillum suwonense; Marinospirillum celere; Clostridium sp.
1001271B 151109 B4; Candidatus Aerophobetes bacterium; Aneurinibacillus sp. BA2021; Sulfobacillus
thermosulfidooxidans; Mesorhizobium; Noviherbaspirillum pedocola; Pedobacter rhizosphaerae; Anaerolineae
bacterium SM23 63; Paralcaligenes sp. KSB-10; Aquisphaera giovannonii; Candidatus Thermofonsia Clade 1
bacterium; Pedobacter roseus; Desulfitibacter sp. BRH c19; Bacillus marinisedimentorum; unclassified
Mesorhizobium; Legionella oakridgensis ATCC 33761 = DSM 21215; Clostridium ihumii; FCB group
bacterium; Luteimonas gilva; Clostridium simiarum; Fontibacillus phaseoli; Clostridium paridis;
Pseudoclostridium thermosuccinogenes; Anaerocolumna aminovalerica; bacterium HR08; Paenibacillus
cineris; Paenibacillus dokdonensis; Pedobacter sp. Leaf216; Bacillus rhizoplanae; Clostridium bornimense;
Flavobacterium sp. HSC-32F16; Papillibacter cinnamivorans; Chryseobacterium profundimaris; Paenibacillus
bouchesdurhonensis; Candidatus Pacearchaeota archaeon; Candidatus Peregrinibacteria bacterium;
Mesorhizobium sp. B2-4-17; Gracilibacteraceae bacterium; Spirochaetia bacterium; Nannochloropsis gaditana;
Candidatus Pacebacteria bacterium; Litoribacterium kuwaitense; Paenibacillus sp. DMB20; Halomonas
azerica; Mesorhizobium sp. INR15; Bacillaceae bacterium JMAK1; Pedobacter sp. HMF7647;
Chitinophagaceae bacterium; Mesorhizobium sp. B2-4-12; Legionella oakridgensis; Noviherbaspirillum soli;
Smithella sp.; candidate division Zixibacteria bacterium RBG-1; Peptoniphilus harei; Anaerovirgula
multivorans; Parcubacteria bacterium DG_72; Paraclostridium bifermentans; Pedobacter sp. SJ11;
Saccharibacillus sp. O16; Paenibacillus sp. PK4536; Pedobacter sp. B4-66; Flavobacterium phragmitis;
Daejeonella rubra; Paenibacillus lemnae; Pedobacter sp. HCMS5-2; Syntrophomonadaceae bacterium;
Paenibacillus albidus; Pseudacidobacterium ailaaui; Paenibacillus cellulositrophicus; Paenibacillus sp. YPG26;
Caulobacter sp.; Maledivibacter halophilus; Clostridium sardiniense; Polyangium aurulentum; Candidatus
Moranbacteria bacterium; Candidatus Dadabacteria bacterium; Chryseobacterium indoltheticum; Legionella
nagasakiensis; Leptospirillum sp. Group IV ‘UBA BS’; Chryseobacterium sp. OSA05B; Chryseobacterium
shigense; Gemmatimonas sp.; Bacillus thuringiensis; Candidatus Microgenomates bacterium; Bryobacteraceae
bacterium; Mesorhizobium sp. B2-4-15; Anaerococcus hydrogenalis; candidate division Kazan bacterium
RBG_13_50_9; Methylovirgula sp. 4M-Z18; Terrabacteria group bacterium ANGP1; Chryseobacterium sp.
MEBOG07; Candidatus Syntrophonatronum acetioxidans; Rhynchophorus ferrugineus; Halonatronomonas
betaini; Paenibacillus sp. S28; Anaerocolumna sp. AGMB13025; Sporobacter termitidis; candidate division
Zixibacteria bacterium RBG_16_40_9; Pedobacter suwonensis; Chromatiales bacterium; Acidobacteria
bacterium OLB17; Geomonas sp. RF6; Chlorobaculum parvum; Gemmatimonadetes bacterium
RIFCSPLOWO2_12_FULL_68_9; Clostridiaceae bacterium 14S0207; Paenibacillus sp.; Lysobacter terrigena;
Nitrospira sp. CG24E; Bacillus wiedmannii; Methylocapsa sp. S129; Pedobacter sp. SYSU D00382;
Gemmatimonadetes bacterium GWC2_71_10; Steroidobacter sp.; Mesorhizobium sp. AR02; Candidatus
Margulisbacteria bacterium GWE2_39_32; Cellulosilyticum ruminicola; Bdellovibrio sp. NC01; Paenibacillus
sp. J22TS3; Eubacteriales bacterium; Tatlockia sp.; Anaerococcus sp. Marseille-P9784; Mesorhizobium sp.
Root102; Mesorhizobium sp. B263B2A; Paenibacillus durus; Chryseobacterium cheonjiense; Candidatus
Thermokryptus mobilis; Steroidobacter soli; Mesorhizobium sophorae; unclassified Flavobacterium;
Flavobacterium crocinum; Terasakiispira papahanaumokuakeensis; Candidatus Bipolaricaulota bacterium;
Paralcaligenes ureilyticus; Clostridium sp. LY3-2; Ignavibacteriales bacterium
CG 4 9 14 3 um filter 30 11; Desulfotomaculum sp. 46 80; Mesorhizobium sp. 113-3-3; Pedobacter
ginsenosidimutans; candidate division WOR-1 bacterium RIFCSPHIGHO2 01 FULL 53 15;
Armatimonadota bacterium; Peptoclostridium acidaminophilum; Bacillus cereus group sp. BfR-BA-01492;
Pelotomaculum sp.; Legionella taurinensis; Chryseobacterium sp. OV279; Chlorobium sp. N1; Paenibacillus
apii; Candidatus Flavonifractor merdavium; Dokdonella sp.; Armatimonadetes bacterium 13 1 40CM 3 65 7;
Mesorhizobium kowhaii; Desulfotomaculum sp.; Fundicoccus ignavus; Desulforamulus ruminis; Polaromonas
sp.; Lucifera butyrica; Mesorhizobium sangaii; Paenibacillus sp. CAA11; Pelotomaculum thermopropionicum
SI; Trueperaceae bacterium; Acidimicrobiales bacterium; Paenibacillus sabinae; Candidatus Kuenenbacteria
bacterium; Siphonobacter curvatus; Prosthecochloris sp. CIB 2401; Paenibacillus lentus; Clostridium
botulinum C; Chryseobacterium sp. Leaf201; Rufibacter sp. XAAS-G3-1; Methylovirgula ligni;
Dethiosulfovibrio peptidovorans; Roseisolibacter agri; Singulisphaera sp. GP187; Ferruginibacter sp.;
Intestinimonas massiliensis; Pusillimonas faecipullorum; Geomonas oryzisoli; Paucimonas lemoignei;
Clostridiales bacterium GWB2_37_7; Candidatus Lokiarchaeota archaeon Loki_b31; Natronincola
peptidivorans; Steroidobacter cummioxidans; Pirellulaceae bacterium; Bacillus cereus group sp.
BfR-BA-01383; Niallia circulans; Chlorobiota bacterium; Fictibacillus enclensis; Bacillaceae; Fusibacter
paucivorans; Deltaproteobacteria bacterium HGW-Deltaproteobacteria-6; Noviherbaspirillum sp. L7-7A;
Anaerococcus ihuae; Flavobacterium sp. S87F.05.LMB.W.Kidney.N; Paenibacillus sp. J23TS9;
Deltaproteobacteria bacterium HGW-Deltaproteobacteria-1; Armatimonadia bacterium; Solimonas aquatica;
Candidatus Saccharibacteria bacterium QS 5 54 17; Ignavibacterium album; Oligoflexia bacterium;
Paenibacillus algicola; Daejeonella lutea; Pedobacter petrophilus; Lewinellaceae bacterium; Candidatus
Buchananbacteria bacterium RIFCSPHIGHO2 02 FULL 38 8; Flavobacterium sp. JLP; Mesorhizobium loti;
bacterium HR36; Bacillus sp. HMF5848; Flavonifractor sp. An82; Mesorhizobium sp.; Noviherbaspirillum
saxi; Anaerococcus porci; Flavobacterium sp. 245; Saccharibacillus sacchari; Nitrospira sp.; Chryseobacterium
sp.; Deltaproteobacteria bacterium RIFCSPHIGHO2 02 FULL 40 28; Chlorobaculum sp. MV4-Y;
Romboutsia lituseburensis; Clostridium kluyveri DSM 555; Candidatus Saccharibacteria bacterium CPR2;
Chryseobacterium sp. 09-1422; Acidimicrobiia bacterium; Owenweeksia hongkongensis; Flavobacterium
reichenbachii; Paracidobacterium acidisoli; Paenibacillus rhizosphaerae; Chryseotalea sanaruensis;
Paludisphaera borealis; Methanomassiliicoccus sp.; Prosthecochloris sp. GSB1; Gemmatimonas aurantiaca
T-27; Gemmatimonas aurantiaca; Clostridium kluyveri NBRC 12016; Massilibacterium senegalense;
Clostridium sp. Ade.TY; Paramaledivibacter caminithermalis; Abyssisolibacter fermentans; Citrifermentans
bemidjiense; Desulfoscipio gibsoniae; Firmicutes bacterium ADurb.Bin373; unclassified Halomonas;
Flavobacterium hydrophilum; Parcubacteria group bacterium Athens0714_25; Calditrichia bacterium;
Calditrichaceae bacterium; Flavobacterium album; Pedobacter yulinensis; bacterium ADurb.Bin429;
Thermanaeromonas sp. C210; Paenibacillus; Burkholderiales bacterium RIFCSPLOWO2_02_FULL_57_36;
Pedobacter namyangjuensis; Candidatus Sumerlaeaceae bacterium; Clostridium kluyveri; Pelosinus
fermentans; Pedobacter sp. MR22-3; Noviherbaspirillum galbum; Thermanaeromonas sp.; Mucilaginibacter
auburnensis; Bacillus cytotoxicus; Peptococcaceae bacterium; Paenibacillus spiritus; Rhodanobacter sp. B04;
candidate division Zixibacteria bacterium RBG_16_48_11; Caulobacter sp. UNC358MFTsu5.1; Acidimicrobiia
bacterium EGI L10123; Armatimonadetes bacterium CG07_land_8_20_14_0_80_40_9; Bacteroidetes
bacterium SW_10_40_5; Spirochaetota bacterium; Hydrogenibacillus sp. N12; bacterium B17; Marivirga sp.
S37H4; Flammeovirgaceae bacterium; Scopulibacillus daqui; Saccharibacillus alkalitolerans; Hippocampus
comes; Runella rosea; Noviherbaspirillum sp. Root189; Clostridium tarantellae; Mesorhizobium sp. B2-3-4;
Candidatus Saccharibacteria bacterium GW2011_GWC2_48_9; miscellaneous Crenarchaeota group archaeon
SMTZ-80; Pelotomaculum propionicicum; Fictibacillus aquaticus; Rhodanobacter sp. C05; Clostridium
botulinum CFSAN002369; Pedobacter sp. Leaf250; Catalinimonas alkaloidigena; Mesorhizobium sp.
LSJC269B00; candidate division CPR3 bacterium 4484_211; Acidobacteria bacterium 13_1_20CM_4_56_7;
Paenibacillus uliginis; Mesorhizobium sp. L2C084A000; Mesorhizobium sp. ES1-4; Pedobacter foliorum;
Fictibacillus nanhaiensis; Chryseobacterium luteum; Pedobacter yonginense; Agrobacterium tumefaciens;
Planomicrobium sp. CPCC 101079; bacterium SGD-2; Flavobacterium sp. KB82; Candidimonas bauzanensis;
Chryseobacterium sp. 7; Terriglobales bacterium; Thermotogota bacterium; Minicystis rosea; Saccharibacillus
qingshengii; Candidatus Kryptonium sp.; Pedobacter endophyticus; Candidatus Tectomicrobia bacterium;
Pedobacter sp. SYSU D00823; Citrifermentans bremense; Mesorhizobium helmanticense; Pelotomaculum
thermopropionicum; Candidatus Saganbacteria bacterium; Chloroflexi bacterium
RBG 19FT COMBO 62 14; Geomicrobium halophilum; Acidisarcina polymorpha; Ectobacillus funiculus;
Flavihumibacter sediminis; Thermoanaerobaculum sp.; unclassified Halanaerobium; Candidatus Flavonifractor
intestinigallinarum; Rufibacter hautae; Geomonas azotofigens; Prosthecochloris sp. HL-130-GSB; Candidatus
Latescibacteria bacterium; Halomonas malpeensis; Membranihabitans marinus; Chryseobacterium
shandongense; Desulfitibacter alkalitolerans; Paenalcaligenes niemegkensis; Desulfotomaculum nigrificans;
Desulforamulus aeronauticus; Anaerococcus urinomassiliensis; Melioribacter roseus; Nitrospiraceae bacterium;
Maledivibacter sp.; Thermanaeromonas toyohensis; Mesorhizobium sp. M8A.F.Ca.ET.057.01.1.1; Spiroplasma
sp. JKS002671; Saccharibacillus kuerlensis; Noviherbaspirillum sp. UKPF54; Peptostreptococcaceae
bacterium; Gemmatimonas phototrophica; Fictibacillus marinisediminis; Ohtaekwangia koreensis; Pedobacter
psychroterrae; Desulfatitalea sp. BRH c12; Selenomonadaceae bacterium; Ignavibacteria bacterium
GWF2_33_9; Fictibacillus sp. 26RED30; Ignavibacteriales bacterium
CG18_big_fil_WC_8_21_14_2_50_31_20; Runella sp. S5

TABLE B
NP_002425.2; XP_016784459.1; NP_001015052.1; NP_001015054.1; PNJ02743.1; XP_025225159.1;
XP 055220685.1; XP 011891187.1; XP 011822258.1; XP 032001168.1; XP 032001167.1;
XP_011822257.1; XP_024649919.1; XP_017716223.1; XP_007978137.2; XP_011822259.1;
XP_032125324.1; XP_035122191.2; XP_012324157.1; XP_017366330.1; XP_032125322.1;
XP_012515057.1; XP_012661237.1; XP_012593217.1; XP_012593216.1; XP_026341206.1;
XP_034526637.1; KAF6490278.1; XP_001494845.3; XP_039079336.1; XP_042779002.1; XP_005621833.1;
XP_020032627.1; XP_005621832.1; XP_005621834.1; XP_025779098.1; XP_042779001.1;
XP_047389408.1; XP_042828520.1; XP_008536632.1; XP_042828518.1; XP_025273023.1;
XP_019504337.1; XP_040315543.1; XP_049495005.1; XP_029805927.1; XP_023102455.1;
XP 036135088.1; CAI9172501.1; KAG5194849.1; XP 055264946.1; XP 016071697.1; XP 047637115.1;
XP 055264947.1; XP 015992259.1; XP 015346550.1; XP 008585505.1; XP 046296760.1;
XP_014442491.1; VFV38375.1; XP_020755858.1; XP_043771623.1; XP_045850005.1; XP_014304681.1;
ELW67741.1; KAF7480741.1; XP_054990167.1; MXQ80907.1; XP_015346551.1; XP_038180989.1;
XP_049623945.1; XP_054442739.1; XP_017895328.1; XP_054990168.1; XP_053410140.1; EPQ10253.1;
XP_005391732.1; XP_022372452.1; KAF5921018.1; XP_022372451.1; EHB14148.1; XP_052053650.1;
XP_006204427.3; AAL32368.1; XP_031209386.1; XP_034361511.1; XP_023567264.1; XP_012924302.2;
ERE69542.1; XP_014398442.1; XP_028749167.1; XP_051023345.1; XP_008155903.2; XP_013843190.1;
XP_044928051.1; XP_028612280.1; MBV99522.1; KAI5228846.1; EPY85320.1; XP_010949223.1;
XP_040604337.1; XP_010949223.2; XP_032768024.1; XP_042524591.1; XP_032316608.1;
XP_031303599.1; XP_036054078.1; XP_014338435.1; XP_037669613.1; XP_010618935.1;
XP_036771927.1; XP_008849009.1; XP_051698168.1; XP_048187913.1; XP_047570167.1;
XP_007955662.1; XP_023416443.1; XP_017523794.2; XP_036857672.1; XP_051036977.1;
XP_045730506.1; XP_012866754.1; XP_037364022.1; XP_036316212.1; XP_040829612.1;
XP_051824617.1; XP_023584137.1; XP_023584136.1; XP_023353523.1; XP_023584135.1;
XP_036595076.1; XP_020862251.1; XP_023413367.1; XP_044513642.1; XP_036595077.1;
XP_031801540.1; XP_027718619.1; XP_007499243.1; XP_006873975.1; XP_021513279.1; ACG63682.1;
XP_043855868.1; XP_004706161.2; XP_006894972.1; XP_013373281.1; XP_037678606.1;
XP 019573642.1; KAF6490275.1; XP 055266486.1; XP 055280219.1; XP 042717497.1; XP 008160760.1;
XP 027718620.1; XP 038274169.1; XP 053898185.1; XP 038274168.1; XP 053898183.1;
XP_043349489.1; XP_034638960.1; XP_030434793.1; XP_032650542.1; XP_048721919.1;
XP_044834924.1; XP_039348635.1; XP_032650540.1; XP_013795360.1; NWR76136.1; XP_032650541.1;
CAD7674994.1; XP_038619306.1; XP_014461253.1; XP_033908328.2; XP_052536393.1; XP_052536391.1;
XP_014429784.1; XP_042683972.1; XP_006124598.1; KYO44572.1; XP_033770015.1; XP_009475529.1;
XP_005990872.1; XP_030068854.1; XP_010717698.1; XP_048817408.1; XP_042717498.1;
XP_005306213.1; RMC17816.1; XP_030338271.1; XP_015732803.1; TRZ24251.1; XP_038007644.1;
XP_009812794.1; NXJ51196.1; KAI1233858.1; XP_051488130.1; XP_041257475.1; XP_040296808.1;
XP_040296809.1; NWQ88916.1; OWK55395.1; KAG8558501.1; XP_044160264.1; XP_044160263.1;
RLW01589.1; XP_040212953.1; GCB80072.1; XP_048847494.1; XP_012825907.2; XP_017952613.2;
XP_023993446.1; XP_041044137.1; XP_018426287.1; XP_029491806.1; KAJ7410026.1; XP_028561546.1;
KAF7243101.1; XP_041433201.1; XP_044187891.1; XP_053550486.1; XP_043945379.1; XP_039630404.1;
XP_043091153.1; XP_053194454.1; XP_053708547.1; XP_053348122.1; XP_036409147.1;
XP_042360152.1; XP_030646734.1; XP_036409146.1; XP_034987466.1; XP_033022399.1;
XP_041633713.1; XP_048102419.1; XP_054849241.1; XP_016143145.1; XP_047663674.1;
XP_045922271.1; KAJ6655979.1; XP_023677646.1; KAJ8345015.1; XP_029293748.1; XP_036422733.1;
XP_044036522.1; MCJ8746490.1; XP_018560545.1; TSZ68964.1; XP_046690522.1; XP_024128040.1;
XP_029913726.1; XP_017310079.1; XP_049601576.1; XP_034075877.1; RVE69714.1; CAG12204.1;
KAE8283351.1; KAJ8251206.1; MCI4392275.1; XP 049417167.1; XP 029913723.1; XP 053468741.1;
XP_048349834.1; XP_026539152.1; XP_020465969.1; EDM04017.1; XP_053468740.1; RXN22755.1;
XP_051881318.1; TRY89240.1; XP_029014714.1; XP_018610296.1; XP_028841468.1; KAG8429658.1;
XP_018610294.1; XP_034297269.1; XP_036945314.1; XP_034047026.1; KAG8006851.1; XP_033501421.1;
KAJ7306076.1; XP_015667436.1; XP_008327456.1; XP_053132311.1; XP_042293839.1; XP_053328103.1;
XP_030264311.1; XP_032086387.1; XP_039216642.1; XP_053328101.1; XP_032877252.1;
XP_039679639.1; XP_028455931.1; NP_001076319.1; XP_054066824.1; KAG8145895.1; XP_020511554.1;
XP_011602413.1; XP_040918728.1; XP_031168853.1; XP_032393364.1; XP_034750113.1; OXB54910.1;
XP_035253115.1; XP_026179884.1; XP_008304671.1; XP_035253113.1; XP_026560323.1;
XP_026851288.2; KAJ1109397.1; XP_013879602.1; KAJ8253921.1; XP_053300099.1; XP_020667655.1;
XP_042188736.1; KAF7225650.1; XP_004071647.2; XP_030236282.1; KAF0032868.1; MBN3276840.1;
GBN10628.1; ROL51177.1; XP_003230501.2; KAG2455860.1; XP_015265378.1; XP_051243433.1;
XP_031420737.1; XP_015215875.1; XP_034567220.1; XP_029692893.1; TWW70145.1; CAI5675877.1;
XP_037544500.1; XP_054717128.1; XP_018610297.1; XP_007433930.1; XP_032832053.1;
XP 032832054.1; XP 033631247.1; XP 023269749.1; XP 016321102.1; GFR00254.1; GFR14856.1;
XP_032461981.1; XP_033631246.1; GFT86630.1; GFY70054.1; XP_035210800.1; KFM56773.1;
XP 030236291.1; GIY84848.1; XP 002734634.1; XP 013420197.1; XP 035696132.1; XP 045603965.1;
XP 045603964.1; VVC45432.1; XP 037798962.1; XP 045603963.1; XP 025425257.1; XP 011437056.2;
XP 027218029.1; XP 022317085.1; XP 052261707.1; XP 045131863.1; XP 005098790.1;
XP 050541131.1; XP 021942413.1; KAJ8037963.1; XP 023218940.1; KAI8501103.1; XP 021942412.1;
KAI8766451.1; XP_033749820.1; PIK42417.1; XP_033749821.1; ELU15282.1; XP_015172118.1;
KAF8773469.1; KAG8183705.1; CAH1794932.1; XP_020906283.1; KAF2899816.1; XP_050848968.1;
XP_048245971.1; XP_011307097.1; KAE9543129.1; XP_026806311.1; XP_011307098.1; XP_014607331.1;
XP_021362657.1; XP_029189721.2; XP_033120183.1; GFG40971.1; XP_046838869.1; RUS81070.1;
XP_015922971.1; KAH9504064.1; CAD7635358.1; CAH1274370.1; CAH1737706.1; XP_025193378.1;
KAJ3664310.1; XP_041363322.1; KAF0767464.1; VDO94504.1; XP_023701425.1; XP_041363451.1;
XP_042142330.1; XP_033606128.1; XP_023701424.1; XP_015117417.1; XP_039312569.1;
XP_020626047.1; MCL4122375.1; KAF5303965.1; XP_025087545.1; XP_046544346.1; XP_024940954.1;
XP_022799137.1; XP_025073356.1; OWF45987.1; XP_034942327.1; XP_048575316.1; XP_044763519.1;
XP_043479324.1; XP_042220361.1; PIK41027.1; CAG5100763.1; CAH1391019.1; XP_022171343.1;
XP_014226303.1; EEC03892.1; XP_032452040.1; CAH1391018.1; XP_045467208.1; KAI0217015.1;
XP_008213416.1; CAH1367731.1; KAJ7386224.1; XP_014276329.1; XP_050422713.1; XP_026465296.1;
KAG8042206.1; XP_018328288.1; XP_050043043.1; XP_008188248.1; RMH14586.1; XP_018666791.1;
XP_034180517.1; EEC03891.1; XP_044089689.1; XP_048576207.1; XP_012531196.1; XP_038069241.1;
XP_029038394.2; XP_022171345.1; XP_022171346.1; NHA14208.1; XP_012219475.1; XP_028413179.1;
XP_027043452.1; CAH1987048.1; XP_048757975.1; XP_012271922.1; KAJ3664309.1; NGP52021.1;
WP_240901301.1; RDD47663.1; XP_043793683.1; XP_002116268.1; XP_002429174.1; XP_012145446.1;
KAH9504065.1; CAH1737707.1; KAG5347081.1; XP_024892153.1; XP_023331833.1; XP_043526823.1;
OQV13712.1; XP_048757974.1; XP_044574840.1; XP_014215010.1; GFG40028.1; XP_042142356.1;
XP_019871875.1; KRZ15991.1; RXG55541.1; XP_043280100.1; XP_011338011.1; XP_011687355.1;
XP_044574839.1; XP_003375670.1; XP_029640826.1; XP_025087562.1; XP_011056122.1;
XP_018403602.1; XP_017884890.1; XP_037509822.1; KAG5713404.1; XP_015438929.1; XP_029677480.1;
VEN33459.1; KAG0425902.1; XP_018020384.1; XP_031837335.1; XP_041473622.1; CAH1099993.1;
XP_046644184.1; KAG5887409.1; XP_008549627.1; XP_054763943.1; XP_018915897.1; RWS22065.1;
XP_967967.1; XP_031554200.1; XP_022080263.1; XP_022080262.1; KAH9371247.1; XP_017752566.1;
XP_014249492.1; WP_240900858.1; XP_033214785.1; XP_046445489.1; XP_037285765.1;
XP_043598397.1; XP_026506571.1; XP_015377141.1; XP_018355523.1; NGX15980.1; XP_033214784.1;
RWS11353.1; CAD7432253.1; MCL4220174.1; XP_011498146.1; XP_018373329.1; CAD7570631.1;
XP_050740050.1; CAB3362084.1; XP_046490543.1; XP_046856270.1; GLG94577.1; XP_046628265.1;
XP_026464104.1; TVQ63452.1; CAG9857745.1; CAB3362083.1; CAD7408308.1; KFD57891.1;
XP_046750752.1; XP_052814312.1; XP_046672794.1; CAD7403950.1; KAI4471329.1; KAF2899815.1;
XP_786488.3; KAF6215819.1; CAD7408307.1; XP_021942418.1; XP_050461441.1; GFO16014.1;
PNF39933.1; XP 023701413.1; CAD7453771.1; XP 019871876.2; XP 021942416.1; CAD7201032.1;
XP 053997133.1; XP 020284364.1; KAG7209764.1; XP 023028411.1; CAD7277595.1; GLG98304.1;
CDW56157.1; XP 018569218.1; XP 044270088.1; XP 023701412.1; KAJ4443954.1; XP 028151354.1;
XP 030766092.1; XP 18047240.1; CAD7241138.1; CAG9822359.1; WP 206211863.1; XP 034242323.1;
KAA0213200.1; XP 049852321.1; XP 011875093.1; XP 049950098.1; XP 050393574.1; XP 018569225.1;
MBI1368773.1; XP_014672378.1; XP_054268013.1; QNN23585.1; OQR70227.1; RMG42883.1;
XP 012171887.3; XP 054155559.1; MCC6677046.1; XP 051161082.1; XP 050393573.1; KAI3359167.1;
XP_033365725.1; CAB3263928.1; XP_014476208.1; MCC7146123.1; XP_046385293.1; KAI5698255.1;
KAJ8667383.1; PSN39652.1; XP_032460867.1; XP_044737807.1; XP_031347571.1; XP_019615571.1;
KAI6652545.1; MBX2851576.1; CAH1281818.1; HCD34427.1; KAJ8038094.1; KAB7495034.1;
KAF2369236.1; VDI69304.1; XP_049799850.1; VDI69305.1; XP_044006431.1; XP_017781484.1;
XP_026281906.1; TVQ48961.1; CAH0547631.1; MBK7405029.1; UTX43260.1; GDX99003.1;
KAH0811256.1; TVS06949.1; KAG5859779.1; MCC6321698.1; XP_019853450.1; EFN81544.1;
CAC5402312.1; ROT84732.1; XP_007605385.1; XP_009264592.1; XP_014276395.1; MCC7205472.1;
EFX88790.1; CAG0998698.1; XP_003739583.1; GAV07795.1; XP_045025554.1; XP_012262224.2;
MCC5862068.1; XP_011144080.1; KAI9564548.1; KAG8222540.1; XP_025110441.1; XP_044223456.1;
NP_586320.1; MAX25709.1; CAH7751284.1; KAH7962001.1; MAY75699.1; XP_042588631.1;
XP_018314358.1; WP_115268644.1; KAA0196133.1; MBX3323842.1; MCC5787853.1; XP_043460798.1;
XP_003073865.1; MAT81836.1; XP_043460797.1; XP_007605579.1; MBI1371276.1; XP_043460795.1;
MCC7409368.1; TRY62801.1; MBL4701465.1; MCL4209960.1; MCC6227892.1; XP_054167211.1;
MBS0196656.1; CAF0845495.1; XP 022905691.1; KAI1303168.1; MCA9306591.1; CAG0973675.1;
CAD7650327.1; MBC7835230.1; MCL4741684.1; TVQ51605.1; MBX3351451.1; NUQ68035.1;
MCG3122578.1; MAE62470.1; KAG4076374.1; MCA9196371.1; XP 011263384.1; KAI4471566.1;
MBX3365941.1; MBX3376569.1; MBX3365424.1; TVQ33698.1; XP 032784219.1; WP 091748845.1;
MBX3362973.1; MCK4872181.1; CAD5112555.1; MCC6428735.1; KAF7490627.2; XP 021958864.1;
OGQ81693.1; XP 046908976.1; RJR30723.1; MCH2154346.1; MBM4108223.1; HBS29806.1;
WP_230742211.1; MCE7972834.1; WP_212903617.1; UXI20054.1; GFQ67279.1; MCR9074446.1;
XP_020284368.1; MBL9001768.1; MCC7192354.1; MBJ93217.1; XP_039290864.1; NNM25430.1;
XP_020284365.1; GJQ29937.1; MCA9304271.1; MBL8745391.1; WP_164800541.1; QYK48446.1;
MCA9280728.1; MBY0112128.1; MCB9845457.1; MCB9847677.1; MBX3404824.1; RZC37530.1;
KAF5304422.1; QKK09513.1; TVQ60234.1; XP_030236299.1; MBM6838090.1; XP_024940956.1;
RMH28342.1; QOI99514.1; XP_032674632.1; MCH8005347.1; WP_093752143.1; XP_043793684.1;
MBX3315857.1; XP_033336230.1; XP_029677489.1; WP_185652882.1; XP_024940955.1; MBP5737488.1;
MBX3392526.1; MBL8964303.1; QQS10476.1; CAD7628785.1; MBO6739829.1; XP_024892152.1;
MBS0188124.1; MCA9287928.1; OIN95852.1; WP_084114959.1; WP_231638353.1; MBZ9686730.1;
MCA9274493.1; HEC33330.1; PYO43024.1; MCE9589536.1; XP_043259089.1; NBC11645.1;
MCC6681845.1; WP_226260660.1; XP_031774959.1; WP_236910817.1; GLI56595.1; MCC6660455.1;
OGQ81321.1; CAG5897951.1; HEY80736.1; MCC7387393.1; WP_242367215.1; WP_261670454.1;
WP_174591786.1; WP_061429332.1; MBX3388764.1; XP_027205096.1; CAD7642519.1; WP_204990525.1;
MBC6954457.1; WP_274292477.1; WP_132379491.1; MBM4042199.1; MAO25048.1; XP_025016883.1;
XP_015786052.1; MBN8597573.1; WP_149733859.1; QDV08608.1; XP_051161073.1; OGZ37962.1;
WP_128211072.1; MBS1105336.1; MAA53098.1; OGY42125.1; XP_011687357.1; XP_012271926.1;
OLC36847.1; MBX3385975.1; SHI77557.1; SCJ61537.1; XP_031364394.1; PIV10374.1; HCT44834.1;
MBY0308484.1; MBG9794905.1; WP_160670170.1; NLC45912.1; NLW41057.1; MCC6951250.1;
MBS0190423.1; MCA9291151.1; WP_197259191.1; WP_072830443.1; MBI3326196.1; MCL1978163.1;
MBL0926033.1; MBL9032369.1; RMD66764.1; RLC39998.1; WP_193418736.1; XP_012062396.1;
HHD92008.1; MCH9057169.1; WP_{——}012036110.1; WP_216302856.1; PYS54000.1; WP_073248251.1;
WP_014405253.1; NLY76915.1; MBD0325789.1; WP_012898785.1; MBL8763102.1; NLD17575.1;
GIW41706.1; WP_103896679.1; KOC69474.1; XP_053203364.1; MBZ9578394.1; EGT3615067.1;
NLF87936.1; WP_054261630.1; WP_239329420.1; PYS41515.1; MAF42745.1; TMB41141.1; HHX12711.1;
WP_129029654.1; XP_018047242.1; RLC98955.1; GIY91558.1; EZA54659.1; WP_176829508.1;
MCD6553490.1; MCD6115309.1; UCD74135.1; MCE2966512.1; WP_216277458.1; WP_078809583.1;
MCH7748195.1; WP_068784404.1; MCA9127743.1; WP_092559944.1; NMB07971.1; UCC64762.1;
MCA1617962.1; MBM4030660.1; MBZ4663795.1; WP_055263583.1; MCD6528622.1; HHT51406.1;
WP_013278049.1; WP_202922757.1; MCC6969354.1; NDI77599.1; MBI2551719.1; WP_227020203.1;
KAJ6222058.1; HDQ34739.1; WP 268039191.1; TMB02216.1; WP 068719165.1; EJO5347840.1;
TMA66795.1; WP_106063558.1; MBI1190664.1; RIL00882.1; MBF6617100.1; PYP52759.1;
XP_029677546.1; OHB21064.1; WP_008908098.1; WP_095177559.1; RLC89339.1; WP_089862934.1;
HBY94031.1; MBP2671468.1; MBV7272392.1; HEB60203.1; WP 089984058.1; MBL8761743.1;
WP 274766984.1; HHU50004.1; KAH0950838.1; WP 084159124.1; XP 033214933.1; RYZ75594.1;
WP 013624138.1; EKE11093.1; MCH8165522.1; NLJ99300.1; WP 012447186.1; WP 054873787.1;
MBC7772629.1; MCH8315553.1; WP 261852950.1; MBE7468524.1; MBU1146249.1; MCQ3974700.1;
TMA49204.1; CAD1473226.1; WP 127576204.1; WP 168179928.1; AKU92512.1; HEY63014.1;
GJM83970.1; MBL0922305.1; WP_246615214.1; MBM4464615.1; TDT72483.1; XP_017797699.1;
MBP5405940.1; MCH8152075.1; PIR01875.1; WP 027630518.1; MBS4026929.1; MBI4836903.1;
WP_251859830.1; WP_127837256.1; WP_183279399.1; WP_200270261.1; WP_072771651.1;
WP_079426545.1; SHN59978.1; PYR95918.1; HIP97136.1; KAF7417593.1; WP_062552570.1;
RMH26294.1; PYP38301.1; WP_014967375.1; WP_050726669.1; CAF1326176.1; MCC7374546.1;
WP_091403152.1; NGM88762.1; MCC6897444.1; WP_151861215.1; MCH7571428.1; XP_043280101.1;
WP_244946990.1; RME11084.1; UYV14214.1; WP_130040192.1; WP_129971401.1; WP_137698044.1;
MBA2627298.1; MCH7848535.1; WP_209020038.1; OQB38947.1; WP_269357957.1; WP_127194599.1;
MBI3335301.1; MBX3380977.1; WP_026882255.1; WP_013741469.1; QNU68739.1; WP_088227517.1;
KAG9463768.1; WP_251579913.1; MCS7060824.1; CAG7833985.1; WP_078025021.1; WP_027623626.1;
HHY26649.1; WP_123052680.1; MBL7075938.1; RME30577.1; WP_050353719.1; WP_202749142.1;
KAI4496517.1; WP_005587313.1; MCD6233286.1; NLV76016.1; WP_007785203.1; TDJ55687.1;
MCH7547420.1; MCK6629438.1; MCA1632151.1; MBI1337948.1; MCL4785899.1; WP_220806198.1;
WP_130007660.1; WP_090551104.1; MCC5830744.1; HHT64961.1; WP_138190752.1; WP_073025196.1;
WP_010076035.1; MCH7797917.1; KPJ71378.1; NLM25810.1; MBA3784845.1; NLY50522.1;
WP_264131648.1; MCC2637519.1; WP_232278650.1; MBM4106754.1; MBI2361352.1; OLD04782.1;
NPA90098.1; WP_018663798.1; WP_206582426.1; HDQ71389.1; PYO52526.1; WP_005488473.1;
TSC72121.1; HHY81685.1; MBA2605300.1; WP 246578088.1; WP 133381733.1; MBO1319816.1;
MCA9286561.1; PYM54917.1; PYO98594.1; RME75644.1; WP 163261621.1; MCH2133231.1;
MBH06728.1; WP 216440752.1; WP 258279069.1; MBR4703082.1; TAH34408.1; HHU79353.1;
MBA2621063.1; RUA03009.1; WP 133531373.1; MBD8499858.1; WP 047170985.1; PSR28342.1;
MBP1743155.1; WP_066678470.1; PBC32966.1; MBX3409232.1; MCC6667894.1; WP_271227724.1;
WP_013738983.1; MCI6276069.1; MBI4517240.1; AIQ44572.1; WP_014185990.1; WP_225445825.1;
MBK6723949.1; MBP9664812.1; OGZ32460.1; MCS7178447.1; MCH2254476.1; MCD6270551.1;
WP_071139351.1; WP_009226811.1; NCA94359.1; WP_221038514.1; WP_066021910.1; MCD6484158.1;
MCB9078614.1; MCD6242889.1; MBN9403123.1; RME34058.1; WP_252130342.1; MBQ7214892.1;
WP_148810639.1; MBP2690163.1; MBI5474552.1; NLI89411.1; MBI3648935.1; MCC5821971.1;
MCB1023794.1; TSC53375.1; WP_169296202.1; WP_202411711.1; PKM81377.1; WP_048081485.1;
MBI4527594.1; PKU36177.1; WP_132028624.1; WP_233695837.1; TMB58773.1; MBE6081100.1;
WP_109714626.1; MYN13408.1; GIP50915.1; MBN1875372.1; NLC42700.1; OOM80105.1;
WP_234117006.1; PYS98413.1; MCU1230778.1; MBE6062575.1; WP_243435971.1; HBB86127.1;
WP_279118650.1; MBC7249445.1; NME99414.1; WP_133516221.1; WP_017416274.1; PYP16026.1;
MBI2402517.1; WP_087431756.1; MAE62895.1; OQC42009.1; MBA4697311.1; MCL4300436.1;
WP_092478394.1; PIR69545.1; RBP46807.1; KAF3420782.1; WP_015327055.1; WP_216415041.1;
MBE7551242.1; MCP4537330.1; NYT59191.1; MCI4445220.1; MBA3443711.1; MBC7225722.1;
MBA2647788.1; WP_018247832.1; TET51458.1; RLC61444.1; WP_091539909.1; NLO46476.1;
WP_246188213.1; WP_021875990.1; UUV17866.1; MCM3869940.1; MCE5222397.1; NOX62846.1;
WP_163237678.1; PIZ90091.1; OYV76697.1; WP_026476103.1; MCA9626698.1; MBX9736552.1;
MBV8299792.1; MCF7885479.1; MBM4118496.1; NLA85541.1; WP_051196348.1; WP_087678506.1;
WP_134117621.1; WP_117522241.1; MBP7377213.1; MCP3902166.1; MBI5576314.1; NLF13123.1;
NLU36854.1; MBI2459678.1; XP_041085183.1; WP_035783131.1; MBM4263964.1; MCL2569392.1;
TFW10202.1; WP_210059978.1; WP_077865853.1; MCS7281965.1; HIU41296.1; NLH31329.1;
SFG23325.1; PYO66729.1; WP_240961381.1; HHW15573.1; MCD6550158.1; MBP1642876.1;
WP_251596377.1; MBK7933728.1; NJD63281.1; WP_054199652.1; MBC8015346.1; MCI0364766.1;
OQX84116.1; WP_027417978.1; NLK36460.1; PYP22401.1; MBV8517737.1; OKZ86273.1; MBP6020273.1;
MBI3414047.1; MBV9209840.1; MBN1955095.1; NQU99780.1; WP_084330854.1; PZN34639.1;
NLE43798.1; WP_040397677.1; MCH8825206.1; MBL0937418.1; WP_145446274.1; MCS7087473.1;
MBQ9902640.1; MBL9136649.1; PYQ64726.1; MCD6521593.1; OPY28901.1; WP_027127889.1;
KAI4499404.1; CDM25097.1; WP_012159016.1; WP_102399493.1; WP_008422804.1; MBA2705073.1;
MBV9620880.1; MBI1852822.1; MSP39821.1; WP_010966252.1; MBR6421744.1; MBB6430921.1;
MCL2827574.1; HHV53998.1; HIR41021.1; NOV87961.1; XP 014248457.1; OMG46231.1;
WP_114370573.1; OGL69065.1; TVX88429.1; TMA94058.1; HBF38381.1; MBY0312470.1;
MBV6273798.1; MCB9103364.1; MBV8545578.1; RLG99179.1; TMK35985.1; WP_261827211.1;
NLN11067.1; HHV45422.1; MBA3334667.1; TMK19505.1; PYP03965.1; WP 261378445.1;
WP 125154684.1; MBV9280475.1; MBD3359307.1; WP 097019371.1; MCC6859981.1; NLL74123.1;
HET89694.1; CDB16267.1; OKP86644.1; TKR56099.1; MCL2367768.1; WP 206151593.1; MCC6533641.1;
NME82333.1; MBC8263297.1; WP 055207239.1; MCA1592874.1; NJM09921.1; GJD11514.1;
MBK9215462.1; NLB41483.1; WP 019850172.1; WP 090395523.1; NLB22297.1; WP 080063508.1;
MBV9215798.1; OGZ32766.1; MBV2181944.1; WP_216156137.1; WP_059048677.1; WP_083612289.1;
MBQ8389468.1; MBX3041565.1; WP 236337573.1; MBP2683725.1; WP 069678481.1; WP 132473398.1;
WP_027338607.1; MBC7791696.1; MBI5417070.1; MBS5037637.1; AUX25053.1; MBM2804531.1;
WP_008826301.1; NRS86771.1; MBD3409240.1; MBO4699008.1; WP_072990027.1; WP_198423085.1;
HFB52463.1; WP_183354800.1; MBE3598845.1; WP_242515294.1; WP_160690060.1; WP_246403261.1;
PYP72262.1; MCF7837491.1; WP_264149663.1; MBM4113669.1; OLC68134.1; WP_072892453.1;
WP_238655861.1; WP_236334795.1; WP_227683755.1; MCA1602785.1; ODM95369.1; WP_245583628.1;
QQR75686.1; MBI4616617.1; GFO59978.1; QLQ05411.1; TAN30479.1; PYO34951.1; HEC06454.1;
MBE3583887.1; WP_014437298.1; WP_168572956.1; WP_095132772.1; WP_218953158.1; MBV9924196.1;
WP_153972893.1; MBW8839172.1; WP_188542643.1; MCC6396956.1; MBL7198787.1; MBK5236158.1;
WP_066874330.1; MBK7580845.1; WP_114490353.1; MBI4475012.1; HBG76488.1; MBN1811280.1;
MBN1922660.1; MBW2237200.1; GIU99696.1; TMA12353.1; WP_003445719.1; MCL5281191.1;
OKP75302.1; MBV9080065.1; UCG89291.1; WP_034849750.1; WP_042360397.1; WP_068679582.1;
NLT42347.1; MBV8369746.1; KAJ3636176.1; MBU5439924.1; NLW08213.1; MCD6080931.1;
NKB87257.1; MBS5315248.1; NLL88646.1; MBN2002489.1; MCR4405566.1; WP_235228933.1;
MBE6948729.1; NLY68132.1; WP 076543453.1; OGZ30788.1; MCA0351070.1; MCL2564081.1;
NIP27196.1; MCB1569085.1; MBN1359515.1; OGV45929.1; MBU3803894.1; PYS69883.1; NLM83887.1;
TFG01810.1; MCC6670508.1; WP 209879504.1; MCH7510832.1; MBI3852464.1; WP 183362498.1;
PZN90524.1; WP 055669232.1; NQU84060.1; MCI7759261.1; MCL4485388.1; WP 042202073.1;
WP 074364995.1; MBI4114303.1; WP 110460258.1; GFO69921.1; MCU1308164.1; WP 048030406.1;
MCJ7459014.1; WP 046506235.1; WP 224035742.1; WP 277472736.1; WP 017525166.1;
WP_278677106.1; MBE3110401.1; KAI7265333.1; WP_211024704.1; MCR4428626.1; MBC7911054.1;
MBC8030606.1; WP_020425645.1; MCL2843082.1; MBP5436084.1; WP_101909648.1; WP_205426792.1;
WP_148337824.1; MBI4470339.1; MCA1577648.1; KUJ61207.1; MBE2280471.1; GBG55416.1;
WP_242847643.1; HDQ22498.1; KAJ7426784.1; OGP34630.1; MBA3661527.1; PYS02629.1; BAU49210.1;
WP_142414719.1; WP_055225420.1; MBM2828976.1; MBP8863310.1; MBW3565377.1; APV50913.1;
MBF8257865.1; PYT06129.1; MBK6589453.1; WP_057979432.1; MCH3965335.1; NFG22394.1;
MBK8248216.1; PVY61913.1; MCI4444656.1; QDU34090.1; MBO4711200.1; MBK8147040.1;
MCS6806380.1; MBC7342782.1; MBC7241223.1; MCE3198113.1; HAF12516.1; HBW50259.1;
MBE7479327.1; NLL17531.1; WP_127067918.1; WP_145077652.1; MBS5884909.1; WP_040542443.1;
HEY76410.1; NPV08023.1; PIU69114.1; MBS0657291.1; WP_253201704.1; MCC7208381.1;
MBP7088961.1; MBM4401764.1; MBA3778205.1; WP_141447891.1; QQS43076.1; OFK81106.1;
WP_171092610.1; MBO9371695.1; PWU22088.1; MBP1646848.1; MBU0474797.1; WP_197703196.1;
WP_092471123.1; WP_068728831.1; MCA2981244.1; WP_075457728.1; MBV9027074.1; MBX7171173.1;
WP_249902101.1; WP_223037694.1; WP_083303719.1; PIU45079.1; MBE6811762.1; HHY35847.1;
MCO6453311.1; WP_237382739.1; MCP4680095.1; MBI2363493.1; KFM94113.1; WP_206102689.1;
MCC6849691.1; MCA9599255.1; WP_102072770.1; MBI4493612.1; WP_058258134.1; MBI1807296.1;
WP_233181196.1; MBI4206544.1; NVM16547.1; KIV51452.1; WP_216450865.1; MCD8211577.1;
MCQ2968635.1; OLD93133.1; MBE6061602.1; MBA2494072.1; WP_072149603.1; NLK21704.1;
MBQ1397564.1; GJQ15876.1; HHY90629.1; WP_055341036.1; KUO76353.1; MBL8887496.1;
WP_104984287.1; NLA87526.1; WP_043664444.1; MCI7691026.1; MCK4519855.1; MBQ9434159.1;
MCM2315453.1; WP_264700532.1; HBL26978.1; KPK05303.1; MBA2334414.1; MBU0704436.1;
MCD6178192.1; MBP7147638.1; NYT65868.1; MBL8980494.1; WP_042218738.1; WP_150235634.1;
WP_106769488.1; WP_077835680.1; MPZ43779.1; WP_249331725.1; MCU1244318.1; OQB44524.1;
MBC7933008.1; NJD09135.1; WP_121443823.1; MCE5245892.1; WP_270647395.1; MCB0691441.1;
MBI4545928.1; MBA3892258.1; WP_207766143.1; QGU94942.1; MBV8725243.1; MBI5066126.1;
WP_235296546.1; OAD60044.1; WP_042231506.1; MBV9959226.1; WP_150275769.1; AIQ38718_1;
NOT48852.1; WP_089749626.1; AXQ31618.1; MBZ4218666.1; NYT81523.1; MBQ6863441.1;
WP_013236885.1; WP_155613500.1; NLM35753.1; RZK21514.1; WP_138209679.1; EKD72108.1;
HHX61530.1; MBC8096944.1; RMD59102.1; KAH9389652.1; MCK9267462.1; MBP9479435.1;
UUM13225.1; WP 114607726.1; MCR4310558.1; KAH0540242.1; MBK7392785.1; MCS7166068.1;
MCL4514871.1; MBQ7263968.1; MCL2763061.1; MCA1590425.1; MBN1659702.1; RKZ28044.1;
WP_205527256.1; MBR3474179.1; TMQ53512.1; TEU16594.1; WP_221803714.1; RMG48518.1;
AIQ66312.1; KKP57765.1; MBM3877837.1; WP 184307252.1; WP 010297031.1; MBN1220360.1;
MBE0569636.1; PUU93680.1; WP 039253595.1; WP 224662908.1; WP 073155376.1; MBP7177838.1;
MBK6515487.1; WP 090040314.1; WP 161810121.1; MBX3357939.1; WP 238417156.1; PRR84240.1;
MBP2673371.1; MCA0375024.1; MCO5095333.1; WP 188499000.1; WP 203165936.1; MCJ7856474.1;
WP 058526080.1; NLT13368.1; AXA36306.1; MBI2213193.1; XP 001025685.1; MBL7989945.1;
MBU0600168.1; NLG63443.1; UZE92930.1; RMF03638.1; OGD23720.1; WP_151637601.1;
MBX3708647.1; MCC7018032.1; OMD38763.1; MBI1966541.1; MCC2667029.1; KKP68865.1;
MAV91806.1; MCO6510053.1; HID63917.1; MBE6071986.1; WP_083675927.1; MBQ9534887.1;
MBV8202933.1; WP_209866367.1; MBA3532139.1; WP_061857102.1; WP_248618273.1; RJP65857.1;
WP_058461528.1; MBS3781910.1; WP_061994293.1; MBF8982352.1; XP_036796953.1; PPE72542.1;
PIV46989.1; WP_089655110.1; HHT36740.1; MCA9149031.1; RMH24471.1; WP_106010061.1;
MYG81815.1; WP_011357084.1; TAL72089.1; WP_071711021.1; MCS6774795.1; HID87996.1;
MCL2558303.1; WP_054955410.1; MBS1794384.1; ENZ29615.1; QQE12148.1; MBQ1275065.1;
NLG76515.1; MBZ9577732.1; MCG6972537.1; MBR6461401.1; MBX3355242.1; MCA1813210.1;
NTU97430.1; MBI3082421.1; KPL08801.1; WP_057579100.1; RMI05071.1; BBA50130.1;
WP_217965740.1; MBV9250693.1; KPL03977.1; TLU58742.1; MBX3384319.1; WP_088535311.1;
WP_133129720.1; MBE3576729.1; WP_128900888.1; NTW78279.1; WP_207425478.1; BAL55034.1;
WP_054537480.1; MBE6023175.1; WP_058293144.1; RME16256.1; MBR6574137.1; MBX5465348.1;
MCE5264489.1; WP_173141090.I; MBA2275890.1; WP_014961429.1; MBL8125362.1; MBD3190878.1;
NLM48758.1; MBA3355997.1; MBL6751543.1; WP_008518324.1; MCU1265627.1; MCE1163986.1;
WP 258605798.1; SEB80022.1; MCC7404932.1; MCB2203714.1; WP 098252338.1; UCD78329.1;
GBC82590.1; KAF5908081.1; MCR4416871.1; MBC5815143.1; WP_077610354.1; WP_139456952.1;
MBP6059657.1; MCA9257724.1; WP 168058325.1; MCC6820266.1; REJ75671.1; RJQ68215.1;
OFO62937.1; WP 205051388.1; NLW25906.1; NOX36824.1; WP 219220884.1; WP 090698789.1;
OIP77463.1; WP_245584876.1; MCD6500583.1; WP_013273278.1; WP_011721816.1; KZK73489.1;
BDE07084.1; WP 165226081.1; PYS95793.1; TMQ57630.1; WP 061612682.1; HCT41220.1;
WP_015247934.1; WP_122638847.1; MBE7514954.1; NDC78974.1; OGT46816.1; MBS4956539.1;
WP_203126634.1; WP_223069335.1; WP_210400918.1; MBI3810930.1; WP_029922036.1; HHW90181.1;
NOZ71182.1; WP_129578573.1; WP_055105445.1; WP_166209404.1; MBL8757574.1; WP_077397608.1;
MBP1656600.1; OOH73632.1; HHX79775.1; WP_250342634.1; MBA2665359.1; MBI5020667.1;
WP_042132721.1; WP_272440820.1; WP_019106905.1; MBL9024965.1; NLC79356.1; MCS6904544.1;
WP_213595547.1; WP_047000818.1; HAU85397.1; MBW2275164.1; MBV8731684.1; MCS7049480.1;
MSV29102.1; WP_269497714.1; MCD6289791.1; UCD15917.1; MCU1309027.1; MCB0275684.1;
TVQ42994.1; OGT43571.1; OIP34757.1; WP_131562885.1; MBK9067681.1; MBW7931683.1;
MBX3020991.1; PYQ30721.1; MCB1161060.1; MBX5480786.1; WP_209701425.1; MCL8208171.1;
MCL4196048.1; PYP54988.1; OGY41777.1; MCI5628058.1; WP_072745160.1; MCD6528612.1;
NIM98768.1; MBV8783137.1; WP_056095747.1; KKS74015.1; TPN34615.1; RYZ06284.1;
WP_066244025.1; WP_213656510.1; NIO11490.1; MCA9283518.1; MCC7549846.1; MBN8727417.1;
MBN1975937.1; KKS91478.1; WP_194723407.1; NBW99213.1; WP_274228206.1; MBR5702463.1;
OGP19056.1; WP_102068556.1; PJB31608.1; MBA3317634.1; MBI3124815.1; MBX7061392.1;
WP_276733348.1; MBZ9572709.1; MCL5953707.1; WP_071637603.1; OQA18497.1; MBV8126958.1;
HHV06838.1; TFJ85644.1; MBN1260978.1; HEY89593.1; WP_267599971.1; CAN96343.1; MCH9014580.1;
MCR4322607.1; MBR5292278.1; OGT35465.1; WP_275935413.1; MCK5086134.1; WP_248624914.1;
WP_200123329.1; WP_250674091.1; MBW6502773.1; WP_144213347.1; MBR5485721.1;
WP_226389319.1; EOR20699.1; MCR4433143.1; NOZ26767.1; WP_188695842.1; MBR3955030.1;
WP_235291028.1; MBM6985669.1; MBN1326130.1; WP_218283615.1; HAK43090.1; KAF0151397.1;
OGT23430.1; WP_205701433.1; PYS32606.1; WP_184171494.1; MBS1645038.1; MBT9281979.1;
WP_184468136.1; MBU91299.1; OPY26440.1; MBV8630427.1; MCI8329159.1; MCB0078514.1;
WP_016113278.1; WP_070967570.1; WP_220179372.1; WP_066892700.1; RKY35266.1; RLE89319.1;
HIE38836.1; MBZ5678235.1; TAJ96682.1; WP_063555521.1; WP_029274354.1; AIA30774.1;
WP_073062089.1; WP_090496264.1; NYT78136.1; UCC87256.1; MCC7487838.1; WP_250345713.1;
MCL6522601.1; NTW56640.1; MBN1507962.1; MBV8265089.1; MBI2839698.1; MCL1951133.1;
OGP09484.1; WP_074657924.1; HHY59166.1; WP_093912856.1; WP_015935392.1; UCE52012.1;
NLN49791.1; OFW78065.1; KTD16249.1; RLG44779.1; UCG35210.1; MBE0643302.1; PZN26995.1;
MCL2128656.1; MCR9102818.1; MBI3182388.1; MBV8244343.1; WP_124847885.1; MCB0265946.1;
TFK07434.1; WP 047800489.1; WP 072723443.1; HHW47326.1; MCR4316839.1; MCC6618973.1;
HBN84858.1; WP_044823451.1; TVM05519.1; MCC7353135.1; MBP1697184.1; TAJ09672.1;
WP_084386375.1; WP_216390537.1; WP_195468087.1; MCB9127901.1; WP_089758804.1;
WP 272561395.1; WP 103374156.1; MBS4030095.1; AKS24695.1; MCC6283726.1; MCM8823472.1;
KKR10019.1; WP 040194434.1; MCL5883828.1; MBP8911302.1; NCQ16241.1; CAG7591085.1;
MCC5910906.1; MBN2169961.1; WP 007202072.1; MBV8491530.1; WP 036724434.1; PYO61056.1;
MBM4358201.1; WP 012978918.1; WP 253350515.1; WP 090043060.1; RMG64568.1; MBD3254879.1;
MBA3522585.1; WP 277644160.1; WP 019639947.1; MBJ6723879.1; WP 215907678.1; WP 246021945.1;
TLY24869.1; MBV8341127.1; WP_023744872.1; KAF0142743.1; WP_054252822.1; MBE3555618.1;
MCA9145836.1; MCD6027436.1; MBP2685379.1; MBN1179205.1; MBP1710577.1; MBK9155277.1;
EET85738.1; WP_028373544.1; MBW4052400.1; TSC63152.1; MBI1815593.1; MCU1348344.1;
SMP64906.1; WP_091961210.1; NLN18902.1; MCK7508137.1; WP_195999563.1; MBC7189982.1;
PYK57976.1; MBX7233738.1; TMB03153.1; NLZ81125.1; MBN6187189.1; WP_053957844.1;
MCC6585618.1; WP_136621782.1; MBX3006900.1; MBV8772433.1; WP_200595167.1; WP_090882512.1;
KPK93981.1; WP_255696721.1; WP_246196355.1; PJF36190.1; WP_187594956.1; CAG0967655.1;
RUA15927.1; TMC98318.1; KUO48860.1; MCF7871121.1; MBC8060216.1; WP_070120596.1;
NLL64100.1; WP_023726779.1; AHE67011.1; WP_040328658.1; TMQ55160.1; MBW6458041.1;
RLG40065.1; MBN2168046.1; RKY44736.1; WP_223266881.1; MBU5591988.1; WP_245955077.1;
WP_202766764.1; AUS95653.1; WP_091687135.1; GBC78163.1; WP_244879535.1; WP_136607614.1;
WP_055913731.1; WP_230575243.1; MCL2139976.1; PID56965.1; MCF2639694.1; MBO2488105.1;
WP_216463051.1; WP_253784995.1; WP_084233707.1; GIO58394.1; MXX33187.1; SMP35281.1;
MBI3609002.1; WP_253294761.1; WP_110929781.1; KYQ47640.1; RUT28078.1; MCB9874972.1;
MBP6908795.1; MCJ7568840.1; RME85912.1; MBI4501240.1; MCK5321572.1; MBP8600934.1;
MBI3331551.1; WP 023525273.1; WP 181168736.1; NLK35783.1; PHV71870.1; MCE1157664.1;
EWM27651.1; MCK5591991.1; WP_151618662.1; WP_165200766.1; MBP1563815.1; WP_046679737.1;
WP 171701960.1; WP 195179520.1; MBK5259782.1; EZH67389.1; WP 160843656.1; PLX32597.1;
MBA2249594.1; WP 181165641.1; WP 025385632.1; MCH7874918.1; MBP7137836.1; WP 194714420.1;
NMC91746.1; EQB63449.1; KXA30763.1; SNS03741.1; KPJ55115.1; WP 150886414.1; MBW3552241.1;
WP 269414633.1; OWA32971.1; MCI0530437.1; WP 273592898.1; MBA3586883.1; MBQ5695322.1;
WP_214226791.1; WP_091495338.1; WP_245704497.1; GJQ61605.1; WP_169506673.1; PYS82461.1;
MCG8540682.1; NLG86134.1; NPV66177.1; WP_269426853.1; MBD1209013.1; MBT9174093.1;
NLH42692.1; WP_041064930.1; WP_229696444.1; MBX6360317.1; NNG16361.1; WP_244364631.1;
WP_251376963.1; WP_098385998.1; MBO9559915.1; WP_079493923.1; WP_221860989.1;
WP_1369231.50.1; WP_000645926.1; HBI17318.1; RMG40534.1; MBR3771371.1; WP_201134445.1;
WP_133127856.1; EQD24757.1; WP_223607829.1; WP_228456258.1; MBO4930252.1; MBA3919334.1;
WP_193413709.1; MBI3984071.1; MCS6954090.1; NTU57420.1; TPK69672.1; MBI3787530.1;
WP_276876808.1; TMQ65905.1; OGB74306.1; WP_116398992.1; MCC6453224.1; MBL0890201.1;
TMJ11024.1; WP_236849965.1; UCB53628.1; RQD77253.1; MCF6270871.1; GFY72294.1; KAF7274075.1;
WP_270453263.1; MBJ9993545.1; WP_278271007.1; MBA3344923.1; WP_073080361.1; MBQ7155737.1;
RME45569.1; KAF0155529.1; MBV5302801.1; MCL4521383.1; RJP61988.1; MBI4101207.1; RPJ13399.1;
MBX2989589.1; OGC78404.1; TMK63772.1; WP_090987245.1; WP_098268474.1; MBL8201731.1;
TET96780.1; MBM4165558.1; MCB9584397.1; MBQ8830930.1; KXK04257.1; MBD5543211.1;
HAV41483.1; MCI9576011.1; MCD4738541.1; MCI1958282.1; MCF8382986.1; WP_230995512.1;
WP_012501607.1; OGU32050.1; AWZ48092.1; MCL6606162.1; TLY71469.1; NLN53922.1; MBI3965483.1;
WP_246022816.1; MBY9002427.1; MBC7928076.1; MBI5214544.1; MBV8067930.1; MCF0147710.1;
WP_039219087.1; PYP18383.1; MCG3149391.1; THJ20627.1; WP_137012809.1; WP_158814994.1;
WP_256006436.1; MBI3802175.1; NLG69483.1; MBI3613965.1; OGT99633.1; KYG11062.1; TAL78490.1;
MBS0037454.1; MBL8265334.1; WP_258588361.1; TFG01008.1; MCA9642629.1; MBV9359514.1;
HEY47771.1; OGI08217.1; WP_054739700.1; WP_142698001.1; MBV8455355.1; WP_244862769.1;
MCA9183294.1; MCE5236249.1; MBA3536642.1; HEC21758.1; MBS1934119.1; NMA54919.1;
PMP96069.1; MBC7360987.1; MCL4427075.1; WP_151409672.1; KQU92052.1; WP_224728674.1;
AIQ10695.1; WP_169230385.1; WP_235894723.1; WP_217705710.1; MTI83882.1; MBI4394904.1;
RLG50316.1; MBA3969371.1; NLK23788.1; WP_095083834.1; WP_244482825.1; WP_264526817.1;
MBK6771443.1; RLE91807.1; MBV9718445.1; HEY42411.1; MBR3059047.1; RYZ87136.1;
WP_109193176.1; RKY54987.1; WP_069000066.1; HAO46512.1; MBS3825915.1; WP_001148759.1;
RME60245.1; WP_243700812.1; MBR4018672.1; MBR9976907.1; WP_042208934.1; WP_257916941.1;
PJB00347.1; KUK65027.1; WP_004814036.1; MCK4383604.1; MBI2604617.1; WP_202357020.1;
HHT70832.1; MBX2995414.1; WP_057934685.1; MBS4804100.1; OGB90356.1; HHF98769.1;
MBY0490012.1; WP 154782579.1; MCD6359959.1; UCD02171.1; WP 025435886.1; WP 242199485.1;
MBK7876764.1; MBQ6207910.1; BCG77296.1; TMK30136.1; NPV74250.1; STY25448.1; SKC83347.1;
GIP24646.1; WP_072923824.1; UCD24252.1; WP_131356466.1; MBM4175359.1; MBV9506819.1;
MBA3959011.1; WP 238324644.1; TLU56739.1; KXA00138.1; WP 165104060.1; HHW06720.1;
HJB99157.1; MBO9662053.1; OLD48313.1; WP 111548052.1; MBE6967807.1; HAG12170.1;
MBK6305092.1; NUQ92933.1; MBI4259298.1; MBY9012917.1; MBI5477736.1; WP 153832592.1;
WP 013842452.1; MBC7671276.1; NYT25000.1; NLN38959.1; WP 122627091.1; MCS7222797.1;
MBD5105614.1; MBX3744282.1; WP 184872802.1; MBV8163365.1; WP 108463819.1; BAF60127.1;
TMQ01146.1; MBW6455630.1; MCU0275052.1; WP_025332649.1; NCO23113.1; WP_104713855.1;
MBE3603946.1; WP 068867852.1; MCR9192249.1; MBL0169239.1; MBP6004017.1; WP 125081600.1;
MCD3245064.1; WP_056220070.1; AUG58453.1; WP_181304597.1; MBK7643173.1; WP_115834715.1;
MAD78307.1; PIE55315.1; MBI5446182.1; GLC28504.1; WP_074304865.1; MCJ7825102.1; MCI6693313.1;
NLU08881.1; NOT92889.1; WP_050617728.1; RLE76115.1; MBA3658231.1; NLP36629.1; NBX77277.1;
WP_226953121.1; MBP2648726.1; MBS1917420.1; MBL7062761.1; MBL8983806.1; MCL5028132.1;
WP_275423163.1; WP_165973787.1; OGO79450.1; TKJ24953.1; WP_090446594.1; WP_116807868.1;
MCC7338284.1; PHV69870.1; HHH83815.1; HHW99441.1; PYV55525.1; WP_242213497.1;
WP_109768775.1; NOX18828.1; WP_061972410.1; WP_031537677.1; WP_213237714.1; PYO86852.1;
HIC89776.1; TET48490.1; PKN19254.1; MBM4194002.1; MBO2478621.1; HHB13388.1; WP_217346214.1;
TCT09792.1; MBC8022709.1; WP_236785190.1; WP_243839394.1; WP_213653266.1; GIU96586.1;
MCM8774206.1; TDX08388.1; PKN85107.1; MBD3174058.1; WP_093289010.1; PSO43001.1;
WP_014559487.1; MBX7137825.1; NMD32878.1; MBL7821018.1; QCT00836.1; WP_079701245.1;
QWV95396.1; WP_154280260.1; TMQ30166.1; PZR58594.1; HCC34060.1; XP_005702867.1;
MCB9275390.1; UCC27186.1; OGY47979.1; MCP4896985.1; WP_233281086.1; MBI5913373.1;
MBP7979088.1; WP 194566344.1; BAV49049.1; MBE6995622.1; MBA2264453.1; MBS3817210.1;
GBD36364.1; MCL5037541.1; WP_125906726.1; MCI9355065.1; MCK4813805.1; MSR85848.1;
MCB0417930.1; WP 087338179.1; TIY09155.1; MBO07203.1; WP 119771354.1; MCK5475765.1;
WP 154539131.1; WP 133524088.1; WP 224689260.1; MBS0578728.1; WP 051507027.1; MBL8072532.1;
RZJ91289.1; MBD3168735.1; OGQ06929.1; MBI4420569.1; WP 260533679.1; MSU76028.1;
MCP5024680.1; CEH34934.1; EDK35729.1; WP 044039286.1; MCE7936971.1; WP 264751773.1;
MCC6339582.1; PYS90121.1; NTV99414.1; WP_014202656.1; WP_035689749.1; WP_117300088.1;
WP_269190145.1; WP_246426965.1; MCL2243841.1; WP_127120906.1; WP_237170742.1; MBI0582604.1;
WP_020373972.1; MBP9106577.1; MCS7235644.1; MCI9586561.1; MBI64889.1; MBQ3087734.1;
RCK75934.1; WP_094083256.1; BAH37697.1; WP_197526041.1; MCH7714533.1; BAH08358.1;
NDC42418.1; MCK4891406.1; WP_062197697.1; MBB3132201.1; MBP7766367.1; MBI4693425.1;
MCS7017112.1; MBD3193662.1; WP_024615341.1; WP_073151358.1; WP_066500405.1; MSR06686.1;
WP_012530547.1; MSQ73494.1; WP_006522384.1; OQA10689.1; WP_096282015.1; MBR5248737.1;
WP_110346093.1; TSD01833.1; RMF37333.1; MBP6772095.1; NRA36053.1; MCK6620459.1; NUQ43289.1;
NIM48118.1; WP_108778369.1; PYO43899.1; WP_107215465.1; OPZ87079.1; OLZ10095.1;
WP_173299151.1; MBV9073566.1; WP_223088495.1; NTV25583.1; OGB22682.1; WP_113652683.1;
MBX7244036.1; MBI3004534.1; WP_041700869.1; MCL2190886.1; MCF7833433.1; MBK8380304.1;
WP_235333940.1; MBK7254673.1; MBX3126588.1; MCL2809964.1; WP_265837405.1; MBY9016348.1;
MBV8144620.1; MCL5259145.1; WP_163968490.1; MCG0277332.1; WP_100341417.1; MBP7341632.1;
TMA46369.1; MBI1957212.1; WP_048721514.1; NLI12027.1; MCI6488445.1; MBD3234295.1;
NLX50488.1; WP_150458240.1; MBS1732519.1; WP_077556911.1; OGC85582.1; MBT9131603.1;
NBQ53616.1; WP_081916455.1; MCO8128272.1; PIU67894.1; PSR04823.1; MCL1991492.1; QZA33405.1;
WP_204259824.1; OVE74398.1; WP_201430073.1; HHL53063.1; WP_239549276.1; UCF41175.1;
WP_166280144.1; HEY52065.1; NLA05579.1; NLN07838.1; MCS7027341.1; XP_019714689.1;
WP_114066199.1; MCB2155583.1; MBT9260311.1; WP_057291367.1; WP_152889815.1; MCU0424922.1;
WP_115619872.1; WP_140733436.1; MBU6434781.1; MCA1571549.1; KKW02676.1; KON27617.1;
WP_243119796.1; WP_094252437.1; TXT66459.1; MCI7474304.1; MBA2633906.1; MCK4380791.1;
WP_077444684.1; NLM52969.1; MCG3211241.1; MXX72885.1; MBS1858462.1; MBN1278801.1;
MBN1583760.1; EPS47825.1; TFG16522.1; WP_231424421.1; WP_277479900.1; MCK4386296.1;
MCG8606126.1; WP_023687504.1; OQX51100.1; OLE12418.1; WP_244562885.1; MBE9478834.1;
WP_023813490.1; WP_224570975.1; WP_173089392.1; MBV9272141.1; WP_222460721.1;
WP_034707168.1; WP_109924457.1; NTE01162.1; MBM3163739.1; MCD6348652.1; NUQ21034.1;
WP_146496826.1; NGR08000.1; RLE78732.1; MCD7948007.1; WP_194139178.1; WP_073107532.1;
MBI5660604.1; WP_121487847.1; MBS3089760.1; MBK7806038.1; MCU1283971.1; MCP5455391.1;
MBK6667317.1; TAJ34997.1; APR81189.1; WP_172200146.1; MCL2265816.1; MCS7229535.1;
GBL39430.1; MBA2669996.1; WP 196099413.1; GIX47441.1; WP 207534076.1; WP 156912168.1;
WP_107651809.1; KUK81625.1; MBI5078809.1; OGO67479.1; WP_184403080.1; WP_236657304.1;
WP_129726591.1; TMB17979.1; MBI2175618.1; MBP2661882.1; MBV8460177.1; NLL29481.1;
MCG7857567.1; MBX3029227.1; MCS7182757.1; WP 114458715.1; MBC7901656.1; HJB81437.1;
WP 149092157.1; HEY71245.1; WP 236026227.1; WP 085660162.1; MBL8861336.1; WP 109667663.1;
UCE02257.1; MCC7489308.1; WP 227389624.1; MBY5957334.1; WP 123853721.1; MBI4372350.1;
MBU5613993.1; VEN38701.1; WP 028308569.1; MXZ36098.1; MBR6779604.1; WP 238663703.1;
MBL8990706.1; WP 003541718.1; MBS0287465.1; MCH7587478.1; WP 072910647.1; WP 073997686.1;
WP_014855562.1; MBX9657955.1; MCT4564876.1; WP_084665579.1; WP_208698576.1; WP_250138036.1;
WP 018978866.1; AXC10966.1; TVP58735.1; MCJ7497287.1; WP 146328520.1; MCL2722569.1;
MCB0722593.1; MBP3928447.1; WP_026850121.1; WP_248253493.1; WP_079686628.1; MBV8498443.1;
NOZ00152.1; WP_131596518.1; PYQ46876.1; KJS29649.1; WP_256635055.1; WP_073487661.1;
MBQ9486846.1; OGU57600.1; MBR2935265.1; MBU6239380.1; WP_053472687.1; WP_197193605.1;
PIQ10094.1; WP_253531503.1

1. A base editor comprising:
(1) a nucleic acid programmable DNA binding domain (napDNAbd) capable of binding a target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
(2) an adenine deaminase domain capable of deaminating the adenine of the target deoxyadenosine to a hypoxanthine; and
(3) a hypoxanthine excising domain capable of excising the hypoxanthine.

2. A system comprising:
(1) a base editor or a polynucleotide encoding the base editor, the base editor comprising:
(a) a nucleic acid programmable DNA binding domain (napDNAbd) capable of binding a target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
(b) an adenine deaminase domain capable of deaminating the adenine of the target deoxyadenosine to a hypoxanthine; and
(c) a hypoxanthine excising domain capable of excising the hypoxanthine; and
(2) a guide nucleic acid or a polynucleotide encoding the guide nucleic acid, the guide nucleic acid comprising:
(a) a scaffold sequence capable of forming a complex with the napDNAbd; and
(b) a guide sequence capable of hybridizing to the target sequence on the target strand of the target dsDNA, thereby guiding the complex to the target dsDNA.

3. A method of modifying a target dsDNA, comprising contacting the target dsDNA with a system, the target dsDNA comprising a target A: T base pair comprising a target deoxyadenosine (dA) (first deoxyribonucleotide) in a protospacer sequence on the nontarget strand (edited strand) of the target dsDNA and a deoxythymidine (dT) (second deoxyribonucleotide) in a target sequence on a target strand (non-edited strand) of the target dsDNA, wherein the protospacer sequence is fully reversely complementary to the target sequence;
the system comprising:
(1) a base editor or a polynucleotide encoding the base editor, the base editor comprising:
(a) a nucleic acid programmable DNA binding domain (napDNAbd) capable of binding the target dsDNA;
(b) an adenine deaminase domain capable of deaminating the adenine of the target deoxyadenosine to a hypoxanthine; and
(c) a hypoxanthine excising domain capable of excising the hypoxanthine; and
(2) a guide nucleic acid or a polynucleotide encoding the guide nucleic acid, the guide nucleic acid comprising:
(1) a scaffold sequence capable of forming a complex with the napDNAbd; and
(2) a guide sequence capable of hybridizing to the target sequence on the target strand of the target dsDNA, thereby guiding the complex to the target dsDNA.

4. The base editor, system, or method of any preceding claim, wherein the target dsDNA is a wild type.

5. The base editor, system, or method of any preceding claim, wherein the target deoxyadenosine is native to the target dsDNA.

6. The base editor, system, or method of any preceding claim, wherein the target deoxyadenosine is a mutation in the target dsDNA.

7. The base editor, system, or method of any preceding claim, wherein the target deoxyadenosine is a pathogenic mutation in the target dsDNA.

8. The base editor, system, or method of any preceding claim, wherein the target dsDNA is a target gene.

9. The base editor, system, or method of any preceding claim, wherein the target deoxyadenosine (first deoxyribonucleotide) is replaced with a fourth deoxyribonucleotide that is different from the target deoxyadenosine (first deoxyribonucleotide) by the base editor.

10. The base editor, system, or method of any preceding claim, wherein the adenine of the target deoxyadenosine is deaminized by the adenine deaminase domain to form a hypoxanthine in situ.

11. The base editor, system, or method of any preceding claim, wherein the hypoxanthine excising domain is substantially capable of excising the hypoxanthine formed in situ by the adenine deaminase domain.

12. The base editor, system, or method of any preceding claim, wherein the hypoxanthine excising domain is substantially capable of cleaving or hydrolyzing the glycosidic bond linking the hypoxanthine formed in situ and the deoxyribose of the target deoxyadenosine.

13. The base editor, system, or method of any preceding claim, wherein the excision of the hypoxanthine formed in situ converts the target deoxyadenosine in the protospacer sequence to an abasic site having the sugar-phosphate backbone of the target deoxyadenosine.

14. The base editor, system, or method of any preceding claim, wherein the target strand is nicked by the napDNAbd.

15. The base editor, system, or method of any preceding claim, wherein the nicking at the target strand induces a deletion in the target strand.

16. The base editor, system, or method of any preceding claim, wherein the dsDNA is in a target cell.

17. The base editor, system, or method of any preceding claim, wherein the deletion at the target strand is repaired, e.g., by translesion synthesis (TLS) in the target cell using the protospacer sequence containing the abasic site as a repair template.

18. The base editor, system, or method of any preceding claim, wherein during the repair of the target strand, a third deoxyribonucleotide (e.g., dG, dA) different from the deoxythymidine (dT) (second deoxyribonucleotide) is formed at the site in the target sequence opposite to the abasic site in the protospacer sequence as a repair template.

19. The base editor, system, or method of any preceding claim, wherein the sugar-phosphate backbone of the target deoxyadenosine at the abasic site is removed, e.g., by an enzyme in the target cell.

20. The base editor, system, or method of any preceding claim, wherein upon the removal of the sugar-phosphate backbone of the target deoxyadenosine at the abasic site, a fourth deoxyribonucleotide (e.g., dC, dT) is formed at the abasic site in the protospacer sequence to base pair with the third deoxyribonucleotide (e.g., dG, dA) in the target sequence, leading to replacement of a target deoxyadenosine to a fourth deoxyribonucleotide (e.g., dA-to-dC, dA-to-dT) in the protospacer sequence.

21. The base editor, system, or method of any preceding claim, wherein the third deoxyribonucleotide is dA, dC, or dG.

22. The base editor, system, or method of any preceding claim, wherein the fourth deoxyribonucleotide is dT, dC, or dG.

23. The base editor, system, or method of any preceding claim, wherein the replacement of the target deoxyadenosine to the fourth deoxyribonucleotide is dA-to-dC, dA-to-dT, or dA-to-dG.

24. The base editor, system, or method of any preceding claim, wherein the replacement converts a stop codon to a non-stop codon or converts a non-stop codon to a stop codon.

25. The base editor, system, or method of any preceding claim, wherein the stop codon is on the sense strand of the dsDNA.

26. The base editor, system, or method of any preceding claim, wherein the replacement occurs on the sense strand or the nonsense strand of the dsDNA.

27. The base editor, system, or method of any preceding claim, wherein the replacement occurs on the sense strand of the dsDNA, converting a stop codon on the sense strand to a non-stop codon or converts a non-stop codon on the sense strand to a stop codon.

28. The base editor, system, or method of any preceding claim, wherein the replacement occurs on the nonsense strand of the dsDNA, converting a stop codon on the sense strand to a non-stop codon or converts a non-stop codon on the sense strand to a stop codon.

29. The base editor, system, or method of any preceding claim, wherein the replacement occurs in the splicing site (e.g., splicing donor, splicing acceptor) of the target dsDNA.

30. The base editor, system, or method of any preceding claim, wherein the replacement occurring in the splicing site (e.g., splicing donor, splicing acceptor) increases or decreases the translation of a transcript transcribed from the target dsDNA.

31. The base editor, system, or method of any preceding claim, wherein the base editor is a fusion protein.

32. The base editor, system, or method of any preceding claim, wherein the base editor comprises, from N-terminal to C-terminal,
(1) the adenine deaminase domain, the napDNAbd, and the hypoxanthine excision domain;
(2) the hypoxanthine excision domain, the adenine deaminase domain, and the napDNAbd; or
(3). the adenine deaminase domain, the hypoxanthine excision domain, and the napDNAbd;
optionally, any two adjacent domains of (1), (2), or (3) are fused with or without a linker.

33. The base editor, system, or method of any preceding claim, wherein the base editor comprises one, two, three, or more hypoxanthine excising domains.

34. The base editor, system, or method of any preceding claim, wherein the hypoxanthine excising domain is substantially capable of or has been engineered to be substantially capable of excising the hypoxanthine.

35. The base editor, system, or method of any preceding claim, wherein the hypoxanthine excising domain comprises a glycosylase or a variant thereof.

36. The base editor, system, or method of any preceding claim, wherein the glycosylase or a variant thereof is substantially capable of or has been engineered to be substantially capable of excising the hypoxanthine.

37. The base editor, system, or method of any preceding claim, wherein the glycosylase is selected from the group consisting of N-methylpurine DNA glycosylase (MPG), 8-oxoguanine DNA glycosylase (OGG1), methyl-CpG binding domain 4, DNA glycosylase (MBD4), thymine DNA glycosylase (TDG), uracil DNA glycosylase (UNG), single-strand-selective monofunctional uracil-DNA glycosylase 1 (SMUG1), mutY DNA glycosylase (MUTYH), nth like DNA glycosylase 1 (NTHL1), nei like DNA glycosylase 1 (NEIL1), nei like DNA glycosylase 2 (NEIL2), nei like DNA glycosylase 3 (NEIL3), and mutants or variants capable of excising the hypoxanthine.

38. The base editor, system, or method of any preceding claim, wherein the hypoxanthine excising domain comprises a N-methylpurine DNA glycosylase protein (MPG).

39. The base editor, system, or method of any preceding claim, wherein the MPG is substantially capable of or has been engineered to be substantially capable of excising the hypoxanthine.

40. The base editor, system, or method of any preceding claim, wherein the MPG substantially has or has been engineered to substantially have N-methylpurine DNA glycosylase activity.

41. The base editor, system, or method of any preceding claim, wherein the MPG comprises a motif GxxYxxxxYGxxxxxN.

42. The base editor, system, or method of any preceding claim, wherein the MPG is obtained from a species selected from Table A.

43. The base editor, system, or method of any preceding claim, wherein the MPG is a variant of an MPG obtained from a species selected from Table A.

44. The base editor, system, or method of any preceding claim, wherein the MPG is a variant of human MPG (SEQ ID NO: 1 or 2) or any MPG as set forth in Table B.

45. The base editor, system, or method of any preceding claim, wherein the MPG comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 1 or 2 or any MPG as set forth in Table B.

46. The base editor, system, or method of any preceding claim, wherein the MPG comprises an amino acid substitution at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, and/or 297 of SEQ ID NO: 2 or a corresponding position of a MPG of another species other than human (e.g., a species selected from Table A other than human), wherein the position is numbered according to the amino acid sequence of SEQ ID NO: 1.

47. The base editor, system, or method of any preceding claim, wherein the MPG comprises an amino acid substitution at position N169 of SEQ ID NO: 2 or a corresponding position of an MPG of another species other than human (e.g., a species selected from Table A other than human), wherein the position is numbered according to the amino acid sequence of SEQ ID NO: 1.

48. The base editor, system, or method of any preceding claim, wherein the amino acid substitution is a substitution with Alanine (Ala/A) or Serine (Ser/S).

49. The base editor, system, or method of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 28; optionally wherein the MPG comprises the amino acid sequence of SEQ ID NO: 28.

50. The base editor, system, or method of any preceding claim, wherein the MPG further comprises an amino acid substitution at position S198, K202, G203, S206, and/or K210 of SEQ ID NO: 28, wherein the position is numbered according to the amino acid sequence of SEQ ID NO: 1.

51. The base editor, system, or method of any preceding claim, wherein the amino acid substitution is a substitution with Alanine (Ala/A).

52. The base editor, system, or method of any preceding claim, wherein the MPG further comprises an amino acid substitution selected from the group consisting of S198A, K202A, G203A, S206A, and K210A relative to SEQ ID NO: 28, wherein the position is numbered according to the amino acid sequence of SEQ ID NO: 1.

53. The base editor, system, or method of any preceding claim, wherein the MPG comprises amino acid substitutions N169S, S198A, K202A, G203A, S206A, and K210A relative to SEQ ID NO: 2, wherein the position is numbered according to the amino acid sequence of SEQ ID NO: 1.

54. The base editor, system, or method of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 30; optionally wherein the MPG comprises the amino acid sequence of SEQ ID NO: 30.

55. The base editor, system, or method of any preceding claim, wherein the MPG further comprises an amino acid substitution at position S78, P79, K80, G81, R110, T115, E116, R120, R138, G163, Q173, G174, D175, A177, E185, L187, E188, L190, E191, T192, Q195, S198, T199, R201, K202, V208, K210, R212, S216, K220, A226, N228, K229, S230, Q238, E240, A241, R246, L249, P251, E253, P254, A255, R272, P274, V279, R280, G281, V291, Q294, D295, T296, Q297, and/or A298 of SEQ ID NO: 28, wherein the position is numbered according to the amino acid sequence of SEQ ID NO: 1.

56. The base editor, system, or method of any preceding claim, wherein the amino acid substitution is a substitution with Arginine (Arg/R) or Lysine (Lys/K).

57. The base editor, system, or method of any preceding claim, wherein the MPG further comprises an amino acid substitution selected from the group consisting of S78R, P79R, K80R, G81R, R110K, T115R, E116R, R120K, R138K, G163R, Q173R, G174R, D175R, A177R, E185R, L187R, E188R, L190R, E191R, T192R, Q195R, S198R, T199R, R201K, K202R, V208R, K210R, R212K, S216R, K220R, A226R, N228R, K229R, S230R, Q238R, E240R, A241R, R246K, L249R, P251R, E253R, P254R, A255R, R272K, P274R, V279R, R280K, G281R, V291R, Q294R, D295R, T296R, Q297R, and A298R relative to SEQ ID NO: 28, wherein the position is numbered according to SEQ ID NO: 1.

58. The base editor, system, or method of any preceding claim, wherein the MPG comprises amino acid substitutions N169S and G163R relative to SEQ ID NO: 2, wherein the position is numbered according to SEQ ID NO: 1.

59. The base editor, system, or method of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 32; optionally wherein the MPG comprises the amino acid sequence of SEQ ID NO: 32.

60. The base editor, system, or method of any preceding claim, wherein the MPG comprises amino acid substitutions N169S, G163R, S198A, K202A, G203A, S206A, and K210A relative to SEQ ID NO: 2, wherein the position is numbered according to the amino acid sequence of SEQ ID NO: 1.

61. The base editor, system, or method of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 34; optionally wherein the MPG comprises the amino acid sequence of SEQ ID NO: 34.

62. The base editor, system, or method of any preceding claim, wherein the adenine deaminase domain comprises a tRNA adenosine deaminase (TadA) or a functional variant or fragment thereof, e.g., TadA8e (SEQ ID NO: 3), TadA8.17, TadA8.20, TadA9, TadA8E^{V106W}, TadA8E^{V106W+D108Q} TadA-CDa, TadA-CDb, TadA-CDc, TadA-CDd, TadA-CDe, TadA-dual, T_{AD}AC-1.2, T_{AD}AC-1.14, T_{AD}AC-1.17, T_{AD}AC-1.19, TAD AC-2.5, T_{AD}AC-2.6, T_{AD}AC-2.9, T_{AD}AC-2.19, T_{AD}AC-2.23, TadA8e-N46L, TadA8e-N46P.

63. The base editor, system, or method of any preceding claim, wherein the adenine deaminase domain comprises an apolipoprotein B mRNA-editing complex (APOBEC) family deaminase, an activation induced deaminase (AID), a cytidine deaminase 1 from Petromyzon marinus (pmCDA1), or a functional variant or fragment thereof, e.g., APOBEC1, APOBEC2, APOBEC3A, APOBEC3B, APOBEC3C, APOBEC3D, APOBEC3F, APOBEC3G, APOBEC3H.

64. The base editor, system, or method of any preceding claim, wherein the adenine deaminase domain comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 3;
optionally, wherein the adenine deaminase domain comprises the amino acid sequence of SEQ ID NO: 3.

65. The base editor, system, or method of any preceding claim, wherein the napDNAbd substantially lacks dsDNA cleavage activity.

66. The base editor, system, or method of any preceding claim, wherein the napDNAbd substantially lacks dsDNA cleavage activity and nickase activity.

67. The base editor, system, or method of any preceding claim, wherein the napDNAbd has nickase activity.

68. The base editor, system, or method of any preceding claim, wherein the napDNAbd has nickase activity to nick the target strand.

69. The base editor, system, or method of any preceding claim, wherein the napDNAbd comprises a Cas nickase or a dead Cas of a Cas protein; optionally wherein the Cas protein is selected from a group consisting of a Cas9 protein (such as, SpCas9, SaCas9, GeoCas9, CjCas9, Cas9-KKH, circularly permuted Cas9, Argonaute (Ago), SmacCas9, Spy-macCas9, xCas9, SpCas9-NG,); a Cas12 protein (such as, Cas12a, AsCas12a, LbCas12a, Cas12b, Cas12c, Cas12d, Cas12e, Cas12f (Cas14), Cas12g, Cas12h, Cas12i, xCas12i, Cas12Max, hfCas12Max, Cas12j, Cas12k, Cas121, Cas12m, Cas12n, Cas120, Cas12p, Cas12q, Cas12r, Cas12s, Cas12t, Cas12u, Cas12v, Cas12w, Cas12x, Cas12y, Cas12z); a Cas13 protein (such as, Cas13a, Cas13b, Cas13c, Cas13d, Cas13e, Cas13f, Cas13x, Cas13y); Csn2; and a mutant thereof; optionally wherein the Cas nickase is a Cas9 nickase (nCas9), such as SpCas9 nickase (SpCas9-D10A); optionally wherein the napDNAbd comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 4; optionally wherein the napDNAbd comprises the amino acid sequence of SEQ ID NO: 4;
optionally wherein the dead Cas is a dead Cas9 (dCas9), such as dead SpCas9 (SpCas9-D10A+H840A); optionally wherein the napDNAbd comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 37; optionally wherein the napDNAbd comprises the amino acid sequence of SEQ ID NO: 37;
optionally wherein the Cas nickase is a Cas12i nickase (nCas12i) or dead Cas12i (dCas12i), such as a deadCas12i of xCas 12i polypeptide; optionally wherein the napDNAbd comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 38; optionally wherein the napDNAbd comprises the amino acid sequence of SEQ ID NO: 38.

70. The base editor, system, or method of any preceding claim, wherein the napDNAbd comprises an IscB nickase (nIscB) or a dead IscB (dIscB) of an IscB protein (e.g., OgeuIscB).

71. The base editor, system, or method of any preceding claim, wherein the napDNAbd comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 4, 37, or 38; optionally wherein the napDNAbd comprise an amino acid sequence of SEQ ID NO: 4, 37, or 38.

72. The base editor, system, or method of any preceding claim, wherein the napDNAbd comprises a TnpB nickase or a dead TnpB of a TnpB protein.

73. The base editor, system, or method of any preceding claim, wherein the base editor comprises an NLS at the N-terminal and/or C-terminal of the napDNAbp.

74. The base editor, system, or method of any preceding claim, wherein the base editor comprises an NLS at the N-terminal and/or C-terminal of the hypoxanthine excising domain.

75. I The base editor, system, or method of any preceding claim, wherein the base editor comprises an NLS at the N-terminal and/or C-terminal of the adenine deaminase domain.

76. The base editor, system, or method of any preceding claim, wherein the NLS is a SV40 NLS, a bpSV40 NLS (e.g., SEQ ID NO: 11 or 12), or a NP NLS (Xenopus laevis Nucleoplasmin NLS, nucleoplasmin NLS).

77. The base editor, system, or method of any preceding claim, wherein the base editor comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 5, 6, 7, 29, 31, 33, and 35; optionally wherein the base editor comprise an amino acid sequence of any one of SEQ ID NOs: 5, 6, 7, 29, 31, 33, and 35.

78. The base editor, system, or method of any preceding claim, wherein the target deoxyadenosine is at a position of the protospacer sequence selected from the group consisting of position 1, position 2, position 3, position 4, position 5, position 6, position 7, position 8, position 9, position 10, position 11, position 12, position 13, position 14, position 15, position 16, position 17, position 18, position 19, position 20, and a combination thereof.

79. The base editor, system, or method of any preceding claim, wherein the target deoxyadenosine is at a position of the protospacer sequence selected from the group consisting of position 3, position 4, position 5, position 6, position 7, position 8, position 9, position 10, and a combination thereof.

80. The base editor, system, or method of any preceding claim, wherein the target deoxyadenosine is at a position of the protospacer sequence selected from the group consisting of position 5, position 6, position 7, position 8, position 9, and a combination thereof.

81. The base editor, system, or method of any preceding claim, wherein the target deoxyadenosine is at position 7 or 8 of the protospacer sequence.

82. The base editor, system, or method of any preceding claim, wherein the target deoxyadenosine is the N₂ nucleotide in a motif of N₁N₂N₃, wherein N₁, N₂, or N₃ is A, T, G, or C; optionally wherein the target deoxyadenosine is the deoxyadenosine (dA) in a motif of CAA or CAG.

83. The base editor, system, or method of any preceding claim, wherein the protospacer sequence comprises about or at least about 16 contiguous nucleotides of the target dsDNA, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target dsDNA, or in a numerical range between any two of the preceding values, e.g., from about 16 to about 50, or from about 17 to about 22 contiguous nucleotides of the target dsDNA; optionally, wherein the protospacer sequence comprises about 20 contiguous nucleotides of the target dsDNA.

84. The base editor, system, or method of any preceding claim, wherein the protospacer sequence is immediately 5′ or 3′ to a protospacer adjacent motif (PAM) comprises sequence 5′-NN-3′, 5′-NNN-3′, 5′-NNNN-3′, 5′-NNNNN-3′, or 5′-NNNNNN-3′, wherein N is A, T, G, or C; optionally wherein the protospacer sequence is immediately 5′ to a protospacer adjacent motif (PAM) comprises sequence 5′-NGG-3′, wherein N is A, T, G, or C; or
optionally wherein the protospacer sequence is immediately 3′ to a protospacer adjacent motif (PAM) comprises sequence 5′-TTN-3′, wherein N is A, T, G, or C.

85. The base editor, system, or method of any preceding claim, wherein the guide sequence is about or at least about 16 nucleotides in length, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any two of the preceding values, e.g., in a length of from about 16 to about 50 nucleotides, or from about 17 to about 22 nucleotides; optionally, wherein the spacer sequence is about 20 nucleotides in length.

86. The base editor, system, or method of any preceding claim, wherein (1) the guide sequence is at least about 50%, 55%, 60%, 65%, 70%, 75%, 80%, 85%, 90%, 95%, or 100% (fully), optionally about 100% (fully), reversely complementary to the target sequence; (2) the guide sequence contains no more than 5, 4, 3, 2, or 1 mismatch or contains no mismatch with the target sequence; or (3) the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence when the PAM is immediately 5′ to the protospacer sequence or at the 3′ end of the guide sequence when the PAM is immediately 3′ to the protospacer sequence.

87. The base editor, system, or method of any preceding claim, wherein the guide sequence comprises a sequence having a sequence identity of at least about 80% (e.g., at least about 80%, 85%, 90%, 91%, 92%, 93%, 94%, 95%, 96%, 97%, 98%, 99%, or 100%) to the sequence of any one of SEQ ID NOs: 40-89; or a sequence having at most 1, 2, 3, 4, 5, 6, 7, 8, 9, or 10 nucleotide differences, whether consecutive or not, compared to the sequence of any one of SEQ ID NOs: 40-89; optionally wherein the guide sequence comprises the polynucleotide sequence of any one of SEQ ID NOs: 40-89.

88. The base editor, system, or method of any preceding claim, wherein the scaffold sequence is 5′ or 3′ to the guide sequence.

89. The base editor, system, or method of any preceding claim, wherein the scaffold sequence is compatible to the napDNAbp.

90. The base editor, system, or method of any preceding claim, wherein the scaffold sequence has substantially the same secondary structure as the secondary structure of SEQ ID NO: 13 or 39;
optionally wherein the scaffold sequence comprises a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of SEQ ID NO: 13 or 39; optionally wherein the scaffold sequence comprises the polynucleotide sequence of SEQ ID NO: 13 or 39.

91. The base editor, system, or method of any preceding claim, wherein the base editor or system further comprises a translesion synthesis (TLS) polymerase or a recruiting domain capable of recruiting a TLS polymerase optionally fused to the base editor, or a coding sequence thereof;
optionally wherein the TLS polymerase is selected from the group consisting of Polα (alpha), Polβ (beta), Polδ (delta) (PCNA), Polγ (gamma), Polη (eta), Pol_{L} (iota), Polκ (kappa), Polλ (lamda), Polμ (mu), Polv (nu), Polθ (theta), and REV1;
optionally wherein the TLS polymerase comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 36; optionally wherein the TLS polymerase comprises the amino acid sequence of SEQ ID NO: 36.
optionally wherein the base editor or system further comprising the translesion synthesis (TLS) polymerase or a recruiting domain capable of recruiting a TLS polymerase leads to replacement of the target deoxyadenosine (first deoxyribonucleotide) with dC, dT, or dG.

92. The base editor, system, or method of any preceding claim, wherein the base editor or system further comprises a cytidine deaminase domain.

93. The base editor, system, or method of any preceding claim, wherein the base editor or system further comprising the cytidine deaminase domain leads to replacement of the target deoxyadenosine (first deoxyribonucleotide) with dT.

94. The base editor, system, or method of any preceding claim, wherein the cytidine deaminase domain facilitates the conversion of the fourth deoxyribonucleotide that is dC to dT.

95. A polynucleotide encoding the base editor of any preceding claim and optionally the guide nucleic acid as defined in any preceding claim.

96. A vector comprising the polynucleotide of any preceding claim.

97. A complex comprising the base editor of any preceding claim and a guide nucleic acid as defined in any preceding claim.

98. A cell comprising the base editor or system of any preceding claim, the polynucleotide of any preceding claim, the vector of any preceding claim, or the complex of any preceding claim.

99. A pharmaceutical composition comprising:
(i) the base editor or the system of any preceding claim, the polynucleotide of any preceding claim, the vector of any preceding claim, the complex of any preceding claim, or the cell of any preceding claim; and
(ii) a pharmaceutically acceptable excipient.

100. A method for treating a subject having or at a risk of developing a disease associated with a target deoxyadenosine of a target dsDNA, comprising administering to the subject (e.g., an effective amount of) the system of any preceding claim, wherein the target deoxyadenosine is modified by the system, and the modification treats or prevents the disease.

101. An MPG substantially capable of or has been engineered to be substantially capable of excising hypoxanthine.

102. The MPG of any preceding claim, wherein the MPG is not wild type human MPG (hMPG; SEQ ID NO: 1), hMPG-N169A, hMPG-N169S, hMPG-N169D, hMPG-N169H, or a variant thereof without N-terminal starting Methionine (M) (e.g., SEQ ID NO: 2).

103. The MPG of any preceding claim, wherein the MPG substantially has or has been engineered to substantially have N-methylpurine DNA glycosylase activity.

104. The MPG of any preceding claim, wherein the MPG comprises a motif GxxYxxxxYGxxxxxN.

105. The MPG of any preceding claim, wherein the MPG is obtained from a species selected from Table A.

106. The MPG of any preceding claim, wherein the MPG is a variant of an MPG obtained from a species selected from Table A.

107. The MPG of any preceding claim, wherein the MPG is a variant of human MPG (SEQ ID NO: 1 or 2) or any MPG as set forth in Table B.

108. The MPG of any preceding claim, wherein the MPG comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 1 or 2 or any MPG as set forth in Table B.

109. The MPG of any preceding claim, wherein the MPG comprises an amino acid substitution at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, and/or 297 of SEQ ID NO: 2 or a corresponding position of a MPG of another species other than human (e.g., a species selected from Table A other than human), wherein the position is numbered according to the amino acid sequence of SEQ ID NO: 1.

110. The MPG of any preceding claim, wherein the MPG comprises an amino acid substitution at position N169 of SEQ ID NO: 2 or a corresponding position of an MPG of another species other than human (e.g., a species selected from Table A other than human), wherein the position is numbered according to the amino acid sequence of SEQ ID NO: 1.

111. The MPG of any preceding claim, wherein the amino acid substitution is a substitution with Alanine (Ala/A) or Serine (Ser/S).

112. The MPG of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 28; optionally wherein the MPG comprises the amino acid sequence of SEQ ID NO: 28.

113. The MPG of any preceding claim, wherein the MPG further comprises an amino acid substitution at position S198, K202, G203, S206, and/or K210 of SEQ ID NO: 28, wherein the position is numbered according to the amino acid sequence of SEQ ID NO: 1.

114. The MPG of any preceding claim, wherein the amino acid substitution is a substitution with Alanine (Ala/A).

115. The MPG of any preceding claim, wherein the MPG further comprises an amino acid substitution selected from the group consisting of S198A, K202A, G203A, S206A, and K210A relative to SEQ ID NO: 28, wherein the position is numbered according to the amino acid sequence of SEQ ID NO: 1.

116. The MPG of any preceding claim, wherein the MPG comprises amino acid substitutions N169S, S198A, K202A, G203A, S206A, and K210A relative to SEQ ID NO: 2, wherein the position is numbered according to the amino acid sequence of SEQ ID NO: 1.

117. The MPG of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 30; optionally wherein the MPG comprises the amino acid sequence of SEQ ID NO: 30.

118. The MPG of any preceding claim, wherein the MPG further comprises an amino acid substitution at position S78, P79, K80, G81, R110, T115, E116, R120, R138, G163, Q173, G174, D175, A177, E185, L187, E188, L190, E191, T192, Q195, S198, T199, R201, K202, V208, K210, R212, S216, K220, A226, N228, K229, S230, Q238, E240, A241, R246, L249, P251, E253, P254, A255, R272, P274, V279, R280, G281, V291, Q294, D295, T296, Q297, and/or A298 of SEQ ID NO: 28, wherein the position is numbered according to the amino acid sequence of SEQ ID NO: 1.

119. The MPG of any preceding claim, wherein the amino acid substitution is a substitution with Arginine (Arg/R) or Lysine (Lys/K).

120. The MPG of any preceding claim, wherein the MPG further comprises an amino acid substitution selected from the group consisting of S78R, P79R, K80R, G81R, R110K, T115R, E116R, R120K, R138K, G163R, Q173R, G174R, D175R, A177R, E185R, L187R, E188R, L190R, E191R, T192R, Q195R, S198R, T199R, R201K, K202R, V208R, K210R, R212K, S216R, K220R, A226R, N228R, K229R, S230R, Q238R, E240R, A241R, R246K, L249R, P251R, E253R, P254R, A255R, R272K, P274R, V279R, R280K, G281R, V291R, Q294R, D295R, T296R, Q297R, and A298R relative to SEQ ID NO: 28, wherein the position is numbered according to SEQ ID NO: 1.

121. The MPG of any preceding claim, wherein the MPG comprises amino acid substitutions N169S and G163R relative to SEQ ID NO: 2, wherein the position is numbered according to SEQ ID NO: 1.

122. The MPG of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 32; optionally wherein the MPG comprises the amino acid sequence of SEQ ID NO: 32.

123. The MPG of any preceding claim, wherein the MPG comprises amino acid substitutions N169S, G163R, S198A, K202A, G203A, S206A, and K210A relative to SEQ ID NO: 2, wherein the position is numbered according to the amino acid sequence of SEQ ID NO: 1.

124. The MPG of any preceding claim, wherein the MPG comprise an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 34; optionally wherein the MPG comprises the amino acid sequence of SEQ ID NO: 34.
